(19) United States
(12) Patent Application Publication
(10) Pub. No.: US 2026/0294887 A1
(43) Pub. Date: Oct. 1, 2026

(54) COMPOSITIONS, METHODS, AND KITS RELATED TO MODULATION OF ZINC TRANSPORTER ZIP12

(71) Applicant: West Virginia University Board of Governors on Behalf of West Virginia University, Morgantown, WV (US)

(72) Inventors: Charles Anderson, Morgantown, WV (US); Werner Geldenhuys, Westover, WV (US)

(21) Appl. No.: 19/629,515

(22) Filed: Mar. 26, 2026

Related U.S. Application Data
(60) Provisional application No. 63/778,507, filed on Mar. 27, 2025.

(51) Int. Cl. A61K 31/454; A61K 31/4709; A61K 31/496; A61K 31/551; A61K 33/30; A61K 38/17; A61K 48/00; C07D 413/04; C07D 413/06; C07D 413/14; C07D 487/14

(52) U.S. Cl. CPC A61K 31/454; A61K 31/4709; A61K 31/496; A61K 31/551; A61K 33/30; A61K 38/1709; A61K 48/005; C07D 413/04; C07D 413/06; C07D 413/14; C07D 487/14

(57) ABSTRACT

Described herein are compounds related to ZIP12 modulation, methods of use, and kits relating to such. In examples, described herein are novel ZIP12 antagonists. In examples, described herein are novel ZIP12 agonists.

### CROSS-REFERENCE TO RELATED APPLICATIONS

This application claims the benefit of U.S. Provisional Application No. 63/778,507 filed on Mar. 27, 2025, which is incorporated herein by reference in its entirety.

### STATEMENT REGARDING FEDERALLY SPONSORED RESEARCH OR DEVELOPMENT

This invention was made with government support under R35 GM138023 awarded by the National Institutes of Health. The government has certain rights in the invention.

### SEQUENCE LISTING

This application contains a sequence listing filed in ST.26 format entitled “52324_1940_sequence_listing.xml” created on Mar. 26, 2026, having 4,661 bytes. The content of the sequence listing is incorporated herein in its entirety.

### BACKGROUND

Synaptic zinc is loaded into presynaptic glutamatergic vesicles by the protein zinc transporter 3 (ZnT3), where it is co-released with glutamate during synaptic transmission. Evidence from clinical studies in humans shows that alterations in the expression of the neuronal zinc transporter protein ZnT3 and the astrocytic zinc transporter protein ZIP12 are associated with schizophrenia, suggesting that dysregulation of these brain-specific zinc transporter proteins may contribute altered synaptic signaling in the brain. There is a need to investigate dysregulation of central-nervous system (CNS) zinc transporters accordingly, as well as address new therapies for CNS disorders, in particular those involving CNS zinc transporter dysregulation accordingly.

### SUMMARY

Described herein are compounds, compositions, methods, and kits relating to ZIP12 modulation. In examples, compounds relating to ZIP12 comprise one or more ZIP12 antagonists. In examples, a ZIP12 antagonist can comprise one of the following structures:

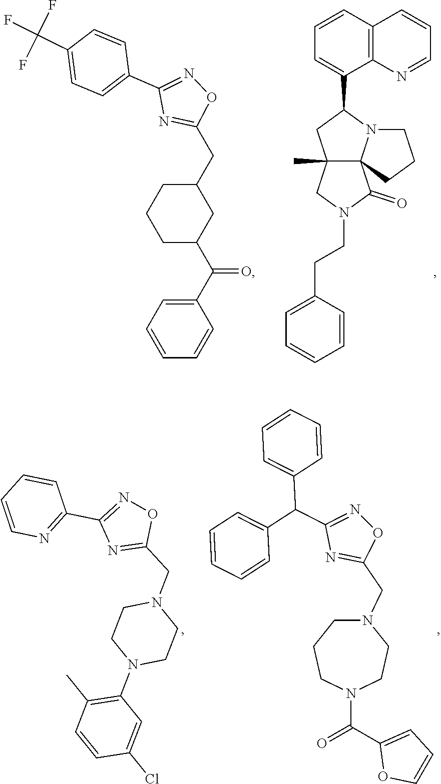

or a pharmaceutically-acceptable salt thereof.

In examples, a pharmaceutical composition is described, comprising one or more ZIP12 antagonists or one or more ZIP agonists, and a pharmaceutically-acceptable carrier.

In examples, described herein are methods of increasing zinc in the synaptic cleft. In examples, a method of increasing zinc in the synaptic cleft comprises administering one or more ZIP12 antagonists described herein, or a pharmaceutically-acceptable salt thereof, to a subject in need thereof.

In examples, the subject in need thereof has, or is suspected of having, dysregulated ZIP12 expression. In examples, the subject in need thereof is a human.

In examples, the one or more ZIP12 antagonists can be administered in an effective amount in increase Zn²⁺ concentration in a synaptic cleft of a subject.

In examples, one or more ZIP12 antagonists can be administered at a concentration of about 1 μM to about 50 μM.

In examples, one or more ZIP12 antagonists can be administered at a concentration of 10 μM.

In examples, a subject in thereof can have one or more of: a neuropsychiatric disorder caused by dysregulated ZIP12 expression, a neurodegenerative disorder caused by dysregulated ZIP12 expression, a neurodevelopmental disorder caused by dysregulated ZIP12 expression, a cancer caused by dysregulated ZIP12 expression, or inflammation caused by dysregulated ZIP12 expression.

In examples, the one or more ZIP12 antagonists can be administered in an effective amount to alleviate one or more symptoms of the neurodegenerative disorder caused by dysregulated ZIP12 expression, the neurodevelopmental disorder caused by dysregulated ZIP12 expression, such as schizophrenia the cancer caused by dysregulated ZIP12 expression such as breast cancer, or the inflammation caused by dysregulated ZIP12 expression.

In examples, a neuropsychiatric disorder caused by dysregulated ZIP12 expression can be schizophrenia. In examples, a neurodegenerative disorder caused by dysregulated ZIP12 expression can be Alzheimer's Disease (AD) or Parkinson's Disease (PD). In examples, a neurodevelopmental disorder caused by dysregulated ZIP12 expression is an autism spectrum disorder (ASD). In examples, a cancer caused by dysregulated ZIP12 expression can be a breast cancer.

In examples, one or more ZIP12 antagonists can be delivered to the central nervous system of the subject. In examples, one or more ZIP12 antagonists can be administered intracerebrally, intraventricularly, or intrathecally.

Described herein are kits. In an example, a kit can comprise one or more ZIP12 agonists or pharmaceutically-acceptable salts thereof, one or more ZIP12 antagonists or pharmaceutically acceptable salts thereof, or both; and instructions for use. In examples, a kit can comprise a polynucleotide vector encoding a functional ZIP12 protein. In examples, one or more ZIP12 agonists, one or more ZIP12, antagonists, or both, can be provided in a dosage unit form. In examples, a dosage unit form can be a concentration of about 1 μM to about 50 μM or about 10 μM. In examples, one or more ZIP12 antagonists can be one or more ZIP12 antagonists described herein. In examples, a kit can further comprise a source of Zn²⁺. In examples, a kit can comprise a negative control. In examples, the negative control can be ZiMo12.9 (N-{1-[5-methyl-1-(2-methylphenyl)-1H-pyrazol-4-yl]ethyl}cyclopropanecarboxamide; CC1=C(C═NN1C1=C(C═CC═C1)C)C(C)NC(═O)C1CC1).

In examples, a ZIP12 agonist can comprise a compound of formula (I):

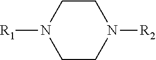

In examples, R₁ and R₂ can be an optionally substituted aromatic ring, an optimally heteroaromatic ring or Ci-substituent, optimally having one or more heteroatoms, C(═O)R, C(═O)OR, or C(═O)NR.

In examples of the compound formula (I), the piperazine can be bonded to R1 or R2 accordingly:

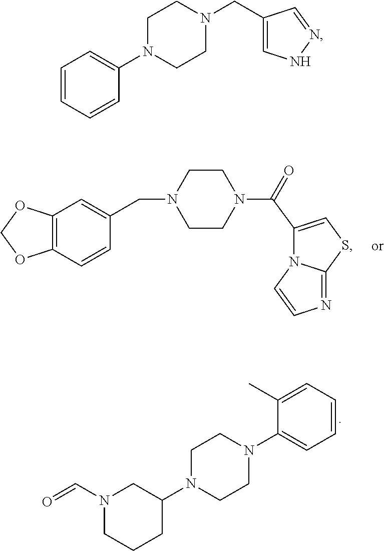 [SMILES: c1ccc(N2CCN(Cc3cn[nH]c3)CC2)cc1 ; O=C(c1csc2nccn12)N1CCN(Cc2ccc3c(c2)OCO3)CC1 ; Cc1ccccc1N1CCN(C2CCCN(C=O)C2)CC1]

In examples, a ZIP12 agonist can comprise one or more of the following compounds of formula (I) (or pharmaceutically-acceptable salt or prodrug thereof):

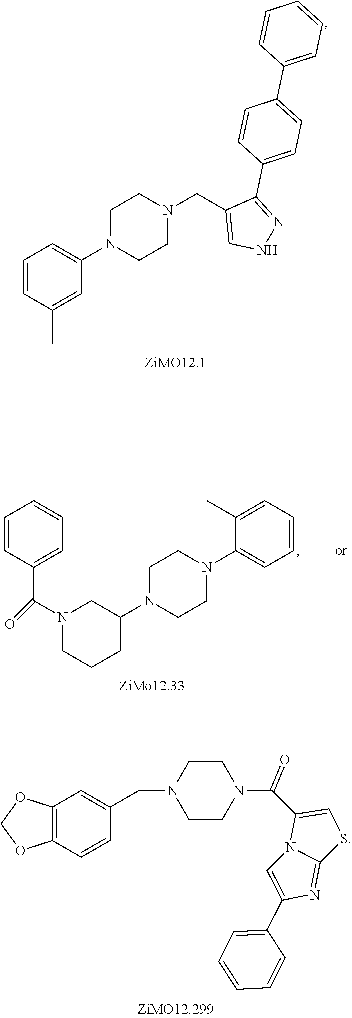 [SMILES: Cc1cccc(N2CCN(Cc3c[nH]nc3-c3ccc(-c4ccccc4)cc3)CC2)c1 ; Cc1ccccc1N1CCN(C2CCCN(C(=O)c3ccccc3)C2)CC1 ; O=C(c1csc2nc(-c3ccccc3)cn12)N1CCN(Cc2ccc3c(c2)OCO3)CC1]

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIGS. 1A-1E. ZIP12 is a synaptic protein. A, Relative gene expression of ZIP12 showing high expression in astrocytes. Also shown are markers for glia (EAAT1), neurons (GAD1, VGLUT1, and bassoon), and vesicles (ZnT3). Images are scatterplot projections of single-cell RNA sequencing data generated from data in Yao et al. (2021), accessed at celltypes.brain-map.org/rnaseq and celltypes.brain-map.org/rnaseq/mouse_ctx-hpf_10x, and displayed in accordance with Allen Institute Transcriptomics Explorer citation policy. B, Example image of bassoon (cyan), ZIP12 (magenta), and EAAT1 (yellow) puncta in Layer 2/3 of mouse ACx. C, Left, Cumulative distribution of the overlap between EAAT1 and bassoon (cyan) puncta and EAAT1 and ZIP12 (magenta) puncta. The cartoon inset shows the volume measured was where the EAAT1 punctum (yellow) and either a ZIP12 (magenta) or bassoon (cyan) punctum overlapped. Right, Average volume overlap for all pairs of puncta natively and with the EAAT1 channel rotated (bassoon, p<1.00×10-15; native, 3,804 pairs; rotated, 2,973 pairs; ZIP12, p<1.00×10-15; native, 1,889 pairs; rotated, 1,309 pairs; n=3 mice; Kruskal-Wallis test with Dunn's multiple comparisons). D, Example image of bassoon (cyan), ZnT3 (orange), and ZIP12 (magenta) puncta in layer 2/3 of mouse ACx. E, Left, Cumulative distribution of the overlap between ZIP12 and bassoon (cyan) puncta and ZIP12 and ZnT3 (orange) puncta. Right, Average volume overlap for both pairs of puncta natively and with the ZIP12 channel rotated (bassoon, p=1.92×10-13; native, 519 pairs, rotated, 109 pairs; ZnT3, p<1.00×10-15; native, 711 pairs; rotated, 80 pairs; n=3 mice; Kruskal-Wallis test with Dunn's multiple comparisons). Asterisks (*) indicate statistically significant values. All values are given as mean±SEM.

FIGS. 2A-2H: ZIP12 and ZnT3 are associated with dendritic spines. A, Top, Example image of corticocollicular neurons (GFP; white), MAP2 (green), and ZIP12 (magenta) in the ACx. Yellow dotted box indicates area imaged for bottom. Bottom, Zoomed in example of corticocollicular neurons and spines (GFP; white), MAP2 (green), and ZIP12 (magenta) in layer 2/3 of the ACx. B, Example image of a synapse in layer 2/3 that does not express any zinc transporter proteins. White outline indicates the dendrite and dendritic spine. Merged image shows ZnT3 (orange) and ZIP12 (magenta) stains. C, Example image of a synapse that only expresses ZnT3. Same color scheme as in B. D, Example image of a synapse that only expresses ZIP12. Same color scheme as in B. E, Example image of a synapse that expresses both ZnT3 and ZIP12. Same color scheme as in B. F, Average spine areas for synapses with no zinc transporters (black), just ZIP12 (magenta), just ZnT3 (orange), and both zinc transporters (purple) (none n=23 spines; ZIP12 n=19 spines; ZnT3 n=24 spines; ZnT3 and ZIP12 n=103 spines in three mice; none-ZIP12, p=3.43×10-3; none-ZnT3, p=2.50×10-3; none-ZnT3 and ZIP12, p=7.00×10-8; ZIP12-ZnT3, p=0.2746; ZIP12-ZnT3 and ZIP12, p=9.17×10-5; ZnT3-ZnT3 and ZIP12, p=3.94×10-4; unpaired t test). G, Pie chart showing the percentage of total synapses with no zinc transporters, just ZIP12, just ZnT3, and both zinc transporters. Same color scheme as G. H, Cartoon showing the tripartite synapses based on the results in A-G. Asterisks (*) indicate statistically significant values. All values are given as mean±SEM.

FIGS. 3A-3K: Identifying small-molecule compounds that reduce ZIP12-mediated zinc fluorescence in HEK293 cells. A, Top, Western blot bands for ZIP12 (top) and GAPDH (bottom) from native and ZIP12 cDNA-transfected HEK293 cells. B, Cartoon depicting the function of FluoZin-3. With the addition of ZnCl2, the cells will fluoresce more. C, Example images of native (top) and transfected (bottom) HEK293 cells with FluoZin-3 after the addition of ZnCl2. D, Example high-throughput zinc uptake assay traces in transfected HEK293 cells with either a vehicle (black) or a test compound (cyan). Examples show a compound with no effect on FluoZin-3 fluorescence (ZiMo12.9) and a compound that reduced FluoZin-3 fluorescence (ZiMo12.8). E, The difference in fluorescence for each test compound compared with the vehicle after the addition of ZnCl2. Cyan labels indicate key compounds. F, Left, Space-filling model of ZIP12 (AF-Q504Y0-F1-v4) Regions 1 and 2 colored blue and red, respectively. Right, Conservation scores of residues located within Region 1 and Region 2 (arbitrary designation) of a putative binding pocket of ZIP12. ConSurf (Ashkenazy et al., 2016; Yariv et al., 2023) run of 150 ZIP12 homologs shows that both regions contain clusters of structurally (s) and functionally (f) conserved residues. G, Docked pose of ZiMo12.8 (stick diagram) in pocket of ZIP12 shown as a space-filling model (gray). H, The binding affinity to the ECD of ZIP12 for each compound tested. Cyan labels indicate key compounds. I, The molecular structure for the four compounds chosen for further experiments. J, Example zinc uptake assay traces of native (black) and ZIP12-transfected (red) HEK293 cells with the prior addition of a vehicle (left) or a compound (right). K, The average fluorescence of FluoZin-3 after the addition of ZnCl2 in native (black) and ZIP12-transfected (red) HEK293 cells. The left bar indicates the addition of a vehicle while the right is the addition of a compound (native vehicle n=6 wells; ZIP12 vehicle n=6 wells; ZiMo12.6 vs ZIP12; p=8.9×10-4; ZiMo12.7 vs ZIP12; p=0.0393; ZiMo12.8 vs ZIP12; p=0.0010; n=6 wells per group; unpaired t test). Asterisks (*) indicate statistically significant values. All values are given as mean±SEM.

FIGS. 4A-4M: ZiMo12.8 increases ZnT3-dependent zinc inhibition of AMPA receptors in mouse ACx. A, Cartoon of a tripartite glutamatergic synapse showing the corelease of zinc and glutamate into the synaptic cleft from the presynaptic neuron, where it acts on AMPA glutamate receptors of the postsynaptic neuron, with an astrocyte expressing ZIP12 clearing zinc from the cleft. B, Example image of an acute brain slice of the inferior colliculus and schematic representation of the injection of the retrograde label cholera toxin subunit B conjugated to an Alexa Fluor 555 fluorophore (CTB-555). C, Example image of an acute brain slice of ACx showing CTB-555 labeled corticocollicular (CCol) neurons in Layer 5. D, Left, Schematic representation of a labeled CCol neuron in patch-clamp configuration. Right, Example image of a labeled CCol neuron in the ACx in whole-cell patch-clamp configuration. E, Left, Example whole-cell patch-clamp recording of AMPA-mediated mEPSCs in acute brain slices of ACx from WT mice in both control conditions (black) and after bath application of ZiMo12.8 (pink). Right, Onset-aligned example mEPSCs from both before (black) and after (pink) treatment with ZiMo12.8. F, Cumulative probability distribution showing AMPA-mediated mEPSC amplitudes normalized to the average amplitude of control mEPSCs in control conditions and after application of ZiMo12.8 in acute brain slices of ACx from WT mice. Colors as in E. G, Bar plot showing the average change in mEPSC amplitude after application of ZiMo12.8, normalized to the average amplitude of control mEPSCs in acute brain slices of ACx from WT mice (WT ZiMo12.8, mean=0.925±0.0295 SEM; p=0.0448; n=7 cells from 5 mice; paired t test). H, Left, Example whole-cell patch-clamp recording of AMPA-mediated mEPSCs in acute brain slices of ACx from ZnT3 KO mice in both control conditions (blue) and after bath application of the novel ZIP12 antagonist ZiMo12.8 (pink). (Right) Onset-aligned example mEPSCs from both before (blue) and after (pink) application of ZiMo12.8. I, Cumulative probability distribution showing the cumulative probability of AMPA-mediated mEPSC amplitudes normalized to the average amplitude of control mEPSCs in control conditions and after application of ZiMo12.8 in acute brain slices of ACx from ZnT3 KO mice. Colors as in H. J, Bar plot showing the average change in mEPSC amplitude after application of ZiMo12.8, normalized to the average amplitude of control mEPSCs in acute brain slices of ACx from ZnT3 KO mice (ZnT3 KO ZiMo12.8, mean=0.919±0.05557 SEM; p=0.2453; n=7 cells from 5 mice; paired ttest). K, Left, Example whole-cell patch-clamp recording of AMPA-mediated mEPSCs in acute brain slices of ACx from WT mice pretreated with ZX1 (yellow) and after bath application of ZiMo12.8 (pink). Right, Onset-aligned example mEPSCs from both before (yellow) and after (pink) treatment with ZiMo12.8. L, Cumulative probability distribution showing the cumulative probability of AMPA-mediated mEPSC amplitudes normalized to the average amplitude of control mEPSCs in control conditions and after application of ZiMo12.8 in acute brain slices of ACx from WT mice pretreated with ZX1. Colors as in K. M, Bar plot showing the average change in mEPSC amplitude after application of ZiMo12.8, normalized to the average amplitude of control mEPSCs in acute brain slices of ACx from WT mice pretreated with ZX1 (WT with ZX1+ZiMo12.8, mean=0.941±0.0826 SEM; p=0.4982; n=4 cells from 3 mice; paired t test). Asterisks (*) indicate statistically significant values. All values are given as mean±SEM.

FIGS. 5A-5I: ZiMo12.8 increases ZnT3-dependent zinc inhibition of NMDA receptors in mouse ACx. A, Cartoon of a tripartite glutamatergic synapse showing the corelease of zinc and glutamate into the synaptic cleft from the presynaptic neuron, where it acts on NMDA glutamate receptors of the postsynaptic neuron, with an astrocyte expressing ZIP12 clearing zinc from the cleft. B, Left, Schematic representation of a layer 5 cortical neuron in patch-clamp configuration. Right, Example image of a layer 5 cortical neuron in the ACx in whole-cell patch-clamp configuration. C, Left, Example patch-clamp recording of the tonic NMDA receptor current from a layer 5 neuron in an acute brain slice of the ACx from WT mice, with the application of ZiMo12.8 (pink) and the NMDAR antagonist AP5 (purple). D, Top, Time course of tonic current recorded from the same cell in C, with the application of ZiMo12.8 (pink) and the NMDAR antagonist AP5 (purple). Bottom, Corresponding time course of capacitance measurements for the same cell. E, Bar plot showing the change in holding current (left) and current density (right) after the application of ZiMo12.8 and AP5 (holding current, ZiMo12.8, mean=0.791±0.00129 SEM; p=3.82×10-5; AP5, mean=0.527±0.0492 SEM; p=0.0107; ZiMo12.8 vs AP5, p=0.034; current density, ZiMo12.8, mean=0.7372±0.017889 SEM; p=0.0046; AP5, mean=0.51641±0.0308 SEM; p=0.0040; ZiMo12.8 vs AP5, p=0.0204; n=3 cells from 3 mice; paired t test). Colors as in D. F, Example patch-clamp recording of the tonic NMDA current from a layer 5 neuron in an acute brain slice of the ACx from WT mice, with the application of the NMDAR antagonist AP5 (purple) before application of ZiMo12.8 (pink). G, Bar plot showing the change in holding current (left) and current density (right) as a measure of tonic NMDA in control conditions and after the application of AP5 and after the application of ZiMo12.8 in AP5 (holding current, WT+AP5, mean=0.71208±0.027692 SEM; p=0.0091; AP5+ZiMo12.8, mean=0.9474±0.041436 SEM; p=0.3392; current density, WT+AP5, mean=0.68877±0.022718; p=0.0053; AP5+ZiMo12.8, mean=1.0112±0.022636 SEM; p=0.6688; n=3 cells from 3 mice; paired t test.) Colors as in D. H, Example patchclamp recording of the tonic NMDA current from a layer 5 neuron in an acute brain slice of the ACx from WT mice, with the application of the ZiMo12.9 (green) and the NMDAR antagonist AP5 (purple). I, Bar plot showing the change in holding current (left) and current density (right) as a measure of tonic NMDA in control conditions, and after the application of ZiMo12.9 and AP5 (holding current, WT+ZiMo12.9, mean=0.96886±0.022698 SEM; p=0.2637; WT+AP5, mean=0.67735±0.074598 SEM; p=0.0228; WT+ZiMo12.9 vs WT+AP5, p=0.0132; current density, WT+ZiMo12.9, mean=1.009±0.020602 SEM; p=0.6910; WT+AP5, mean=0.54374±0.11247 SEM; p=0.0270; WT+ZiMo12.9 vs WT+AP5, p=0.0262; n=4 cells from 4 mice; paired t test). Colors as in H, Asterisks (*) indicate statistically significant values. All values are given as mean±SEM.

FIGS. 6A-6B: FIG. 6A shows an example experimental protocol to study ZIP12 agonists according to the present disclosure. FIG. 6B highlights the agonist activity of compounds from a hit screen described herein.

FIGS. 7A-7C: FIG. 7A displays representative examples of ZIP12 agonists according to the present disclosure. FIGS. 7B and 7C show activity of an example of FIG. 7A.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, cellular biology, immunology and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

### Definitions

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject-matter.

The term “about”, when used herein in reference to a value, refers to a value that is similar, in context to the referenced value. In general, those skilled in the art, familiar with the context, will appreciate the relevant degree of variance encompassed by “about” in that context, for example, ±5%, ±4%, ±3%, ±2%, etc.

Two events or entities are “associated” with one another, as that term is used herein, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

As used herein, the term “comparable” refers to two or more agents, entities, situations, sets of conditions, etc., that may not be identical to one another but that are sufficiently similar to permit comparison there between so that one skilled in the art will appreciate that conclusions can reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Those skilled in the art will appreciate that the term “composition”, as used herein, can be used to refer to a discrete physical entity that comprises one or more specified components. In general, unless otherwise specified, a composition can be of any suitable form—e.g., gel, liquid, solid, etc.

A composition or method described herein as “comprising” one or more named elements or steps is open-ended, meaning that the named elements or steps are essential to a particular aspect or embodiment, but other elements or steps can be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and can also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step can be substituted for that element or step.

In this disclosure, “consisting essentially of” or “consists essentially” or the like, when applied to methods and compositions encompassed by the present disclosure refers to compositions and methods like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “improved,” “increased” or “reduced,” or grammatically comparable comparative terms, indicate values that are relative to a baseline value or reference measurement. For example, in some embodiments, an assessed value achieved with an agent of interest may be “improved” relative to that obtained or expected in the absence of treatment or with a comparable reference agent or control. Alternatively, or additionally, in some embodiments, an assessed value achieved with an agent of interest may be “improved” relative to that obtained in the same subject or system under different conditions (e.g., prior to or after an event such as administration of an agent of interest), or in a different, comparable subject (e.g., in a comparable subject or system that differs from the subject or system of interest). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein, “isolated” means separated from constituents that otherwise may be present, for example, separated from bacterial stains or species that are not desired, or separating from other constituents that may be present with a micro-organism or cell in nature.

As used herein, the term “encode” refers to principle that DNA can be transcribed into RNA, which can then be translated into amino-acid sequences that can form proteins

As used herein, the terms “optional” or “optionally” means that the subsequently described event or circumstance can or cannot occur, and that the description includes instances where said event or circumstance occurs and instances where it does not.

As used herein, “individual”, “organism”, “host”, “subject”, and “patient” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). These terms (“individual,” “subject,” “host,” and “patient,” used interchangeably herein also refer to any mammalian subject for whom diagnosis, treatment, or therapy is desired, particularly humans. In embodiments, subject may relate to particular components of the subject, for instance specific tissues or fluids of a subject (e.g., human tissue in a particular area of the body of a living subject), which may be in a particular location of the subject, referred to herein as an “area of interest” or a “region of interest.”

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

Reference throughout this specification to “one embodiment”, “an embodiment”, “another embodiment”, “some embodiment,” means that a particular feature, structure or characteristic described in connection with the embodiment is included in at least one embodiment of the present disclosure. Thus, appearances of the phrases “in one embodiment,” “in an embodiment,” “in another embodiment”, or “in some embodiment” in various places throughout this specification are not necessarily all referring to the same embodiment, but they may. Furthermore, the particular features, structures or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the disclosure. For example, in the appended claims, any of the claimed embodiments can be used in any combination.

A “control” sample or value refers to a sample that serves as a reference, usually a known reference, for comparison to a test sample or condition. For example, a test sample can include cells exposed to a test condition or a test agent, while the control is not exposed to the test condition or agent (e.g., negative control). The control can also be a positive control, e.g., a known primary cell or a cell exposed to known conditions or agents, for the sake of comparison to the test condition. A control can also represent an average value gathered from a plurality of samples, e.g., to obtain an average value. For therapeutic applications, a sample obtained from a patient suspected of having a given disorder or deficiency can be compared to samples from a known normal (non-deficient) individual. A control can also represent an average value gathered from a population of similar individuals, e.g., patient having a given deficiency or healthy individuals with a similar medical background, same age, weight, etc. A control value can also be obtained from the same individual, e.g., from an earlier-obtained sample, prior to the disorder or deficiency, or prior to treatment. One of skill will recognize that controls can be designed for assessment of any number of parameters.

The term “biological sample” encompasses a variety of sample types obtained from an organism or a cell line. The term encompasses blood and other liquid samples of biological origin, solid tissue samples, such as a biopsy specimen or tissue cultures or cells derived therefrom and the progeny thereof. The term includes samples that have been manipulated in any way after their procurement, such as by treatment with reagents, solubilization, or enrichment for certain components. The term includes a clinical sample, and includes cells in cell culture, cell supernatants, cell lysates, serum, plasma, biological fluids, and tissue samples.

As used throughout, the terms “nucleic acid,” “nucleic acid sequence,” “oligonucleotide,” “nucleotides,” “polynucleotides,” or other grammatical equivalents as used herein mean at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, cRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

Unless otherwise indicated, a particular nucleic acid sequence also implicitly encompasses conservatively modified variants thereof, alleles, orthologs, SNPs, and complementary sequences as well as the sequence explicitly indicated.

As used herein, “cDNA” refers to a DNA sequence that is complementary to an RNA transcript in a cell. It is a man-made molecule. Typically, cDNA is made in vitro by an enzyme called reverse-transcriptase using RNA transcripts as templates.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “gene” can refer to a hereditary unit corresponding to a sequence of DNA that occupies a specific location on a chromosome and that contains the genetic instruction for a characteristic(s) or trait(s) in an organism. The term “gene” can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein, the term “recombinant” generally refers to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc.). Recombinant also refers to the polypeptide encoded by the recombinant nucleic acid. Non-naturally occurring nucleic acids or polypeptides include nucleic acids and polypeptides modified by man.

As used herein, “gene construct” or “construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc. Described herein are polynucleotide vectors that encode a functional ZIP12 protein. Vectors described herein may be suitable for viral packaging and subsequent viral transformation of a cell, for example, a mammalian cell or mammalian glial cell.

The terms “transformation” and “transfection” mean the introduction of a nucleic acid, e.g., an expression vector, into a recipient cell including introduction of a nucleic acid to the chromosomal DNA of said cell.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88).

The terms “polypeptide” and “peptide” are used interchangeably herein to refer to a polymer of amino-acid residues in a single chain. The terms apply to amino-acid polymers in which one or more amino-acid residue is an artificial chemical mimetic of a corresponding naturally occurring amino acid, as well as to naturally occurring amino-acid polymers and non-naturally occurring amino-acid polymers. Amino-acid polymers may comprise entirely L-amino acids, entirely D-amino acids, or a mixture of L- and D-amino acids. The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions. The terms “portion” and “fragment” are used interchangeably herein to refer to parts of a polypeptide, nucleic acid, or other molecular construct.

The term “amino acid” refers to naturally occurring and synthetic amino acids, as well as amino-acid analogs and amino-acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino-acid analogs refer to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino-acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23(4): 581-87 (2013); Xie et al. “Adding amino acids to the genetic repertoire,” Curr. Opin. Chem. Biol. 9(6): 548-54 (2005); and all references cited therein. Beta and gamma amino acids are known in the art and are also contemplated herein as unnatural amino acids.

In accordance with standard nomenclature, amino-acid residue sequences are denominated by either a three letter or a single letter code as indicated as follows, for example: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be involved in the structure, function, and regulation of various functions.

The term “chimeric molecule” refers to a single molecule created by joining two or more molecules that exist separately in their native state. The single, chimeric molecule has the desired functionality of all of its constituent molecules. One type of chimeric molecules is a fusion protein.

The term “conservative amino-acid substitution” refers to the interchangeability in proteins of amino-acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino-acid substitution groups are: valine-leucine-isoleucine, phenylalanine-tyrosine, lysine-arginine, alanine-valine, and asparagine-glutamine.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino-acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith & Waterman Add. APL. Math. 2:482 (1981), by the homology alignment algorithm of Needleman & Wunsch J. Mol. Biol. 48:443 (1970), by the search for similarity method of Pearson & Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-10 and Altschul et al. (1977) Nucleic Acids Res. 25: 3389-402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1977)). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino-acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino-acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino-acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

As used herein, the term “biologically active fragments” or “bioactive fragment” of the polypeptides encompass natural or synthetic portions of the full-length protein that are capable of specific binding to their natural ligand or of performing the function of the protein.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “vector” is a composition of matter which comprises a nucleic acid and which can be used to deliver the nucleic acid to the interior of a cell.

Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, poly amine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, an “essentially pure” preparation of a particular protein or peptide is a preparation wherein at least about 95%, and preferably at least about 99%, by weight, of the protein or peptide in the preparation is the particular protein or peptide.

A “subsequence”, “fragment” or “segment” is a portion of an amino acid sequence, comprising at least two or more amino acids, or a portion of a nucleic acid sequence comprising at least two or more nucleotide. The terms “subsequence”, “fragment” and “segment” are used interchangeably herein.

The terms “polypeptide fragment” or “fragment”, when used in reference to a particular polypeptide, refers to a polypeptide in which amino-acid residues are deleted as compared to the reference polypeptide itself, but where the remaining amino-acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the 25 positions, e.g., 9 of 10, are matched or homologous, the two sequences share 90% homology. By way of example, the DNA sequences 3′ ATTGCC5′ and 3′TATGGC share 50% homology.

As used herein, “homology” is used synonymously with “identity.”

An “isolated polypeptide” refers to a polypeptide, or segment or fragment thereof, which has been separated from a naturally occurring state and/or that is present in a substantially purified form. In some embodiments, an isolated polypeptide refers to a polypeptide that has been isolated from one or more substances otherwise present in an artificial reaction by which the polypeptide is produced or employed (e.g., an in vitro expression reaction).

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell and/or which might be otherwise present in an artificial reaction by which the nucleic acids are produced or employed. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light scattering.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino-acid sequence” includes all nucleotide sequences that are degenerate versions of each other and that encode the same amino-acid sequence. Nucleotide sequences that encode proteins and RNA may include introns.

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

A “disease” is a state of health of an animal wherein the animal (i.e., a mammal such as a human) cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. A “condition” encompasses both diseases and disorders.

The terms “co-administration” or “co-administered” as used herein refer to the administration of a composition described herein along with any one or more reagents that are utilized in diagnosis and/or treatment. In some embodiments, the co-administration is concurrent. In other embodiments, a composition or reagent is administered prior to a second composition or reagent in this aspect, each component may be administered separately, but sufficiently close in time to provide the desired effect. Those of skill in the art understand that the formulations of the various enzymes whose use is described herein may vary. The appropriate dosage for co-administration can be readily determined by one skilled in the art.

The term “composition” as used herein refers to a product comprising the specified ingredients (i.e., one or more enzymes described herein) in the specified amounts, as well as any product which results, directly or indirectly, from combination of the specified ingredients in the specified amounts. Such a term in relation to a composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

A composition of the disclosure can be a liquid solution, suspension, emulsion or a powder. Compositions described herein may include sterile aqueous or non-aqueous solvents, such as water, isotonic saline, isotonic glucose solution, buffer solution, or other solvents conveniently used for parenteral administration of therapeutically active agents, stabilizers, buffers, or preservatives, e.g. antioxidants such as methylhydroxybenzoate or similar additives.

A composition of the disclosure may be sterilized by, for example, addition of sterilizing agents to the composition, irradiation of the composition, or heating the composition. Alternatively, the compounds or compositions of the present disclosure may be provided as sterile solid preparations e.g. lyophilized powder, which are readily dissolved in sterile solvent immediately prior to use.

The term “freeze-dried (lyophilized) as used herein refers to a preparation of a component described herein, for example, nucleic acids or enzymes, that have been initially frozen and the water content removed by vacuum.

The term “reducing” means to diminish in extent, amount, or degree.

As used herein, an “isolated nucleic acid molecule”, “isolated polynucleotide”, and “isolated nucleic acid fragment” may be used interchangeably and refer to a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

As used herein, the term “codon optimized”, as it refers to genes or coding regions of nucleic acid molecules for transformation of various hosts, refers to the alteration of codons in the gene or coding regions of the nucleic acid molecules to reflect the typical codon usage of the host organism without altering the polypeptide for which the DNA codes. As used herein, “synthetic genes” can be assembled from oligonucleotide building blocks that are chemically synthesized using procedures known to those skilled in the art. These building blocks are ligated and annealed to form gene segments that are then enzymatically assembled to construct the entire gene.

As used herein, the term “chemically synthesized”, as pertaining to a DNA sequence, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequences to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

The term “variant” refers to an amino-acid or peptide sequence having conservative amino-acid substitutions, non-conservative amino-acid substitutions (i.e., a degenerate variant), substitutions within the wobble position of each codon (i.e., DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

In some embodiments, suitable polypeptides may include clonotype sequences comprising an amino-acid sequence having at least 30%, 33%, 40%, 50%, 60%, 70%, 80%, 85%, 90%, 91%, 92%, 93%, 94%, 95%, 96%, 97%, 98%, or 99% sequence identity to any of the amino-acid sequences reported herein.

In some embodiments, suitable isolated nucleic acid molecules encode a protein having an amino-acid sequence that is at least about 20%, preferably at least 30%, 33%, 40%, 50%, 60%, 70%, 80%, 85%, 90%, 91%, 92%, 93%, 94%, 95%, 96%, 97%, 98%, or 99% identical to the amino-acid sequences reported herein. Suitable nucleic acid molecules not only have the above homologies, but also typically encode a protein having about 210 to 380 amino acids in length, about 300 to about 360 amino acids, preferably about 310 to about 350 amino acids, and most preferably about 320 to about 335 amino acids in length wherein each protein is characterized as having lyase activity.

The terms “isolated”, “purified”, or “biologically pure” as used herein, refer to material that is substantially or essentially free from components that normally accompany the referenced material in its native state (in particular free from its naturally-occurring state).

The term “clinical well-being” as used herein, refers to a state or degree of clinical or physiological wellness or health of a patient. A clinician can evaluate a patient's clinical well-being by physical examination or performing one or more tests or assays.

“Inhibitors,” “activators,” and “modulators” of expression or of activity are used to refer to inhibitory, activating, or modulating molecules, respectively, identified using in vitro and in vivo assays for expression or activity of a described target protein (or encoding polynucleotide), e.g., ligands, agonists, antagonists, and their homologs and mimetics. The term “modulator” includes inhibitors and activators. Inhibitors are agents that, e.g., inhibit expression or bind to, partially or totally block stimulation or protease inhibitor activity, decrease, prevent, delay activation, inactivate, desensitize, or down regulate the activity of the described target protein, e.g., antagonists. Activators are agents that, e.g., induce or activate the expression of a described target protein or bind to, stimulate, increase, open, activate, facilitate, enhance activation or protease inhibitor activity, sensitize or up regulate the activity of described target protein (or encoding polynucleotide), e.g., agonists. Modulators include naturally occurring and synthetic ligands, antagonists and agonists (e.g., small chemical molecules, antibodies and the like that function as either agonists or antagonists). Such assays for inhibitors and activators include, e.g., applying putative modulator compounds to cells expressing the described target protein and then determining the functional effects on the described target protein activity, as described above. Samples or assays comprising described target protein that are treated with a potential activator, inhibitor, or modulator are compared to control samples without the inhibitor, activator, or modulator to examine the extent of effect. Control samples (untreated with modulators) are assigned a relative activity value of 100%. Inhibition of a described target protein is achieved when the activity value relative to the control is about 80%, optionally 50% or 25, 10%, 5% or 1%. Activation of the described target protein is achieved when the activity value relative to the control is 110%, optionally 150%, optionally 200, 300%, 400%, 500%, or 1000-3000% or higher.

The terms “administering,” “delivering,” and “introducing,” can be used interchangeably to indicate the introduction of a diagnostic or therapeutic composition or agent (e.g., compositions comprising one or more agent described herein) into the body of a subject. The therapeutic composition or agent can be administered through any appropriate means that results in the delivery of at least a portion of the composition or agent to a desired location in the subject such that the composition or agent retains its therapeutic capability. Useful methods of delivering the therapeutic include, but are not limited to, intravenous delivery, subcutaneous delivery, intradermal delivery, intracoronary delivery, intracardiac delivery, oral delivery, or any combination thereof.

The term “administered continuously” refers to the continuous delivery of a therapeutic agent, e.g., compound, molecule, peptide, biologic, chemical, etc. over a 24-hour period or more.

The term “therapeutically effective amount” refers to an amount of therapeutic agent effective to treat at least one symptom of a disease or disorder in a subject, for example, a symptom related to an HIV infection. In other words, such an amount is sufficient to bring about a beneficial or desired clinical effect. The “therapeutically effective amount” of the agent for administration may vary based upon the desired activity, the diseased state of the subject being treated, the dosage form, method of administration, subject factors such as the subject's sex, genotype, weight and age, the underlying causes of the condition or disease to be treated, the route of administration and bioavailability, the persistence of the administered agent in the body, evidence of natriuresis and/or diuresis, the type of formulation, and the potency of the agent.

As used herein, the terms “pharmaceutically acceptable” or “pharmacologically acceptable” refer to compositions that do not substantially produce adverse reactions, e.g., toxic, allergic, or immunological reactions, when administered to a subject.

A “pharmaceutically acceptable excipient,” “pharmaceutically acceptable diluent,” “pharmaceutically acceptable carrier,” or “pharmaceutically acceptable adjuvant” means an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use. “A pharmaceutically acceptable excipient, diluent, carrier and/or adjuvant” as used in the specification and claims includes one and more such excipients, diluents, carriers, and adjuvants.

As used herein, a “pharmaceutical composition” is meant to encompass a composition or pharmaceutical composition suitable for administration to a subject, such as a mammal, especially a human. In general, a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, intravenous, buccal, rectal, parenteral, intraperitoneal, intradermal, intracheal, intramuscular, subcutaneous, inhalational and the like.

The terms “therapy,” “treatment,” and “amelioration” refer to any reduction in the severity of symptoms, e.g., of a disease, for example, an HIV infection. As used herein, the terms “treat” and “prevent” are not intended to be absolute terms. Treatment can refer to any delay in onset, amelioration of symptoms, improvement in patient survival, improved cognitive function or coordination, increase in survival time or rate, etc. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

As used herein, a “chemically modified” amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

A “spacer” as used herein refers to a peptide that joins the proteins comprising a fusion protein. Generally, a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically, specific delivery results in a much stronger association between the delivered molecule and cells bearing the target molecule than between the delivered molecule and cells lacking the target molecule.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the IgG class. In addition to intact immunoglobulin molecules, also included in the term “antibodies” are fragments or polymers of those immunoglobulin molecules, and human or humanized versions of immunoglobulin molecules that selectively bind the target antigen.

The term “protein domain” refers to a portion of a protein, portions of a protein, or an entire protein showing structural integrity; this determination may be based on amino-acid composition of a portion of a protein, portions of a protein, or the entire protein.

The term “direct bond” refers to a chemical bond such as a covalent bond or an ionic bond.

The term “substituted” refers to any one or more hydrogens on the designated atom that can be replaced with a selection from the indicated group, provided that the designated atom's normal valence is not exceeded, and that the substitution results in a stable compound. When a substituent is keto (i.e., C—C(═O)—C), then 2 hydrogens on the atom can be replaced. Keto substituents are not present on aromatic moieties. When a ring system (e.g., carbocyclic or heterocyclic) is said to be substituted with a double bond, it is intended that the carbonyl group or double bond be part of the ring.

The term “aliphatic group” refers to a saturated or unsaturated linear or branched hydrocarbon group and encompasses alkyl, alkenyl, and alkynyl groups, for example.

The terms “alkyl” or “alkyl group” as used herein refer to a saturated aliphatic hydrocarbon radical which can be straight or branched, having 1 to 20 carbon atoms, wherein the stated range of carbon atoms includes each intervening integer individually, as well as sub-ranges. Examples of alkyl include, but are not limited to methyl, ethyl, n-propyl, i-propyl, n-butyl, s-butyl, t-butyl, n-pentyl, s-pentyl, and nonyl. The term “lower alkyl” means an alkyl group having less than 10 carbon atoms.

The terms “alkenyl” or “alkenyl group” as used herein refer to an aliphatic hydrocarbon radical which can be straight or branched, containing at least one carbon-carbon double bond, having 2 to 20 carbon atoms, wherein the stated range of carbon atoms includes each intervening integer individually, as well as sub-ranges. Examples of alkenyl groups include, but are not limited to, ethenyl, propenyl, n-butenyl, i-butenyl, 3-methylbut-2-enyl, n-pentenyl, heptenyl, octenyl, decenyl, and the like.

The term “arylalkyl” refers to an arylalkyl group wherein the aryl and alkyl are as herein described. Examples of arylalkyl include, but are not limited to, -phenylmethyl, -phenylethyl, -phenylpropyl, -phenylbutyl, and -phenylpentyl.

The term “substituted,” as in “substituted alkyl”, “substituted cycloalkyl,” “substituted cycloalkenyl,” substituted aryl, “substituted biaryl,” “substituted fused aryl” and the like means that the substituted group may contain in place of one or more hydrogens a group such as hydroxy, amino, halo, trifluoromethyl, cyano, —NH(lower alkyl), —N(lower alkyl)₂, lower alkoxy, lower alkylthio, or carboxy, and thus embraces the terms haloalkyl, alkoxy, fluorobenzyl, and the sulfur and phosphorous containing substitutions referred to below.

The terms “halo”, “halogen”, or “halogen radical” as used herein refer to a fluorine, chlorine, bromine, and iodine, and radicals thereof. Further, when used in compound words, such as “haloalkyl” or “haloalkenyl”, “halo” refers to an alkyl or alkenyl radical in which one or more hydrogens are substituted by halogen radicals. Examples of haloalkyl include, but are not limited to, trifluoromethyl, trichloromethyl, pentafluoroethyl, and pentachloroethyl.

The term “alkoxy” represents an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. The term “lower alkoxy” means an alkoxy group having less than 10 carbon atoms.

The term “cycloalkyl” refers to a non-aromatic mono- or multicyclic ring system of about 3 to about 10 carbon atoms, preferably of about 5 to about 10 carbon atoms. Advantageous ring sizes of rings of the ring system include about 5 to about 6 ring atoms. Exemplary monocyclic cycloalkyl include cyclopentyl, cyclohexyl, cycloheptyl, and the like. Exemplary multicyclic cycloalkyl include 1-decalin, norbornyl, adamant-(1- or 2-)yl, tricyclo[3.3.1.1^{3.7}]decane, and the like.

The term “cycloalkenyl” refers to a non-aromatic mono- or multicyclic ring system of about 3 to about 10 carbon atoms, preferably of about 5 to about 10 carbon atoms, and which contains at least one carbon-carbon double bond. Advantageous ring sizes of rings of the ring system include about 5 to about 6 ring atoms. Examples of monocyclic cycloalkenyl include cyclopentenyl, cyclohexenyl, cycloheptenyl, and the like. An exemplary multicyclic cycloalkenyl is norbornylenyl.

The term “aryl” as used herein, refers to an aromatic monocyclic or multicyclic ring system of about 6 to about 14 carbon atoms, preferably of about 6 to about 10 carbon atoms. Exemplary aryl groups include phenyl or naphthyl, or phenyl substituted or naphthyl substituted.

The term “heteroaryl” is used herein to denote an aromatic ring or fused ring structure of carbon atoms with one or more non-carbon atoms, such as oxygen, nitrogen, and sulfur, in the ring or in one or more of the rings in fused ring structures. Examples are furanyl, pyranyl, thienyl, imidazyl, pyrrolyl, pyridyl, pyrazolyl, pyrazinyl, pyrimidinyl, indolyl, quinolyl, isoquinolyl, quinoxalyl, and quinazolinyl.

The term “biaryl” refers to an aryl, as defined above, where two aryl groups are joined by a direct bond or through an intervening alkyl group, preferably a lower alkyl group.

The term “fused aryl” refers to a multicyclic ring system as included in the term “aryl,” and includes aryl groups and heteroaryl groups that are condensed. Examples are naphthyl, anthryl and phenanthryl. The bonds can be attached to any of the rings.

The terms “aralkyl” and “heteroaralkyl” as used herein refer to aryl and heteroaryl moieties, respectively, that are linked to a main structure by an intervening alkyl group, e.g., containing one or more methylene groups.

The term “fluorobenzyl” refers to a benzyl group wherein the phenyl moiety is substituted with one or more fluorine atoms, including 2, 3, 4 and 5 fluorine atom substituents.

Similarly, “halobenzyl” refers to benzyl substituted with one or more different halogens, including fluorine, chlorine, bromine, and iodine (not astatine).

The terms “sulfide” and “thioether” as used herein, alone or in combination, refer to a sulfur atom covalently linked to two atoms; the formal oxidation state of said sulfur is (II). These terms may be used interchangeably.

The term “sulfanyl” as used herein, alone or in combination, refers to the —S—R group, wherein R may be a group such as: alkyl, alkenyl, alkynyl, aryl, alicyclic, heterocyclic, aryl, heteroaryl, arylalkyl and heteroarylalkyl, wherein the alkyl, alkenyl, alkynyl, aryl, alicyclic, heterocyclic, aryl, heteroaryl, arylalkyl and heteroarylalkyl groups may be optionally substituted. Non-limiting examples of sulfanyl groups include methylsulfanyl (—SCH₃) and iso-propylsulfanyl (—SCH(CH₃)₂) and the like.

The term “sulfoxide” as used herein, alone or in combination, refers to a sulfur atom covalently linked to three atoms, at least one of which is an oxygen atom; the formal oxidation state of said sulfur atom is (IV).

The term “sulfinyl” as used herein, alone or in combination, refers to the groups —S(O)—R, wherein R may be, but is not limited to alkyl, alkenyl, alkynyl, aryl, alicyclic, heterocyclic, aryl, heteroaryl, arylalkyl and heteroarylalkyl, wherein the alkyl, alkenyl, alkynyl, aryl, alicyclic, heterocyclic, aryl, heteroaryl, arylalkyl and heteroarylalkyl groups may be optionally substituted. A non-limiting example of a sulfinyl group includes methylsulfinyl (—S(O)CH₃) and the like.

The term “sulfone” as used herein, alone or in combination, refers to a sulfur atom covalently linked to four atoms, at least two of which are oxygen atoms; the formal oxidation state of said sulfur atom is (VI).

The term “sulfonyl” as used herein, alone or in combination, refers to the groups —S(O₂)—R, wherein R may be, but is not limited to alkyl, alkenyl, alkynyl, aryl, alicyclic, heterocyclic, aryl, heteroaryl, arylalkyl and heteroarylalkyl, wherein the alkyl, alkenyl, alkynyl, aryl, alicyclic, heterocyclic, aryl, heteroaryl, arylalkyl and heteroarylalkyl groups may be optionally substituted. A non-limiting example of a sulfonyl group includes methylsulfonyl (—S(O₂)CH₃) and the like.

The term “phosphine” as used herein, alone or in combination, refers to a phosphorus atom covalently linked to at least one carbon atom, wherein the formal oxidation state of said phosphorus is (III).

The term “phosphinyl” as used herein, alone or in combination, refers to the monoradical derived from a phosphite group, as defined above.

The term “phosphonate” as used herein, alone or in combination, refers to a phosphorus atom covalently linked to four atoms, three of which are oxygen and one of which is carbon wherein the formal oxidation state of said phosphorus is (V).

The term “phosphonyl” as used herein, alone or in combination, refers to the monoradical derived from a phosphonate group, as defined above.

The term “phosphate” as used herein, alone or in combination, refers to a phosphorus atom covalently linked to four oxygen atoms, wherein the formal oxidation state of said phosphorus is (V).

The term “phosphatidyl” as used herein, alone or in combination, refers to the monoradical derived from a phosphate group, as defined above.

The terms ketone, ester, ether, and acyl have their art recognized meanings.

To the extent that the disclosed compounds, and salts thereof, may exist in their tautomeric form, all such tautomeric forms are contemplated herein as part of the present disclosure.

All stereoisomers of the compounds of the present disclosure, such as those that may exist due to asymmetric carbons on the various substituents, including enantiomeric forms (which may exist even in the absence of asymmetric carbons) and diastereomeric forms, are contemplated within the scope of this disclosure. Individual stereoisomers of the compounds of the disclosure may, for example, be substantially free of other isomers, or may be admixed, for example, as racemates or with all other, or other selected, stereoisomers. The stereogenic centers of the compounds of the present disclosure can have the S or R configuration as defined by the IUPAC 1974 Recommendations.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salts” refers to a compound of the present disclosure that can be modified by making acid or base salts thereof. Pharmaceutically acceptable salt refers to those salts that retain the biological effectiveness and optionally other properties of the free bases and that are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like. Examples of pharmaceutically acceptable salts include, but are not limited to, mineral or organic acid salts of basic residues such as amines; and alkali or organic salts of acidic residues such as carboxylic acids.

In the event that embodiments of the present disclosure form salts, these salts are within the scope of the present disclosure. Reference to an agent of any of the formulas herein is understood to include reference to salts thereof, unless otherwise indicated. The term “salt(s)”, as employed herein, denotes acidic and/or basic salts formed with inorganic and/or organic acids and bases. In addition, when an agent contains both a basic moiety and an acidic moiety, zwitterions (“inner salts”) may be formed and are included within the term “salt(s)” as used herein. Pharmaceutically acceptable (e.g., non-toxic, physiologically acceptable) salts are advantageous, although other salts are also useful, e.g., in isolation or purification steps which may be employed during preparation. Salts of the compounds of an agent may be formed, for example, by reacting the agent with an amount of acid or base, such as an equivalent amount, in a medium such as one in which the salt precipitates or in an aqueous medium followed by lyophilization.

Embodiments of the present disclosure that contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Embodiments of the agents that contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine, and the like.

### DISCUSSION

The present disclosure describes novel small molecule (i.e., less than 2500 dalton) modulators of zinc transporter ZIP12 (i.e., ZIP-12). Zinc transporter ZIP12 is a protein that is encoded by the solute carrier 39 member 12 (SLC39A12) gene (e.g. Mus musculus UniProtKB/Swiss-Prot: Q5FWH7.1; Homo sapiens NCBI Reference Sequence: NP_001138667.1 (Isoform 1)). ZIP12 dysregulation and/or dysfunction is noted in a number of neuropsychiatric, neurodevelopmental, and neurodegenerative disorders, cancers and inflammation. ZIP12 is a novel target for such diseases, and small molecules targeting the ZIP12 zinc transporter can help modulate zinc, especially synaptic zinc that is normally regulated by astrocytic ZIP12, for example.

As demonstrated by the present disclosure, ZIP12 can be expressed in astrocytes, which can have projections (i.e., as astrocytic process, for example) that can surround or otherwise form part of a neuronal synapse. Normal astrocyte function helps maintain homeostasis and synaptic transmission, however, dysregulation of transporters such as ZIP12 can impair the ability of the astrocyte to buffer the synapse and maintain stasis.

Described herein are novel ZIP12 antagonists, novel ZIP12 agonists, pharmaceutically-acceptable salts thereof, pharmaceutical compositions thereof, methods of use, and kits relating to such.

### I. COMPOUNDS AND COMPOSITIONS RELATED TO ZIP12 MODULATION

Described herein are compounds and compositions comprising compounds related to ZIP12 modulation. In examples, described herein are ZIP12 antagonists and pharmaceutically-acceptable salts thereof. In examples, described herein ZIP12 agonists and pharmaceutically-acceptable salts thereof.

ZIP12 modulators (i.e., antagonists and agonists), can utilize any of the following chemical scaffolds with substitutions according to the present disclosure, for example:

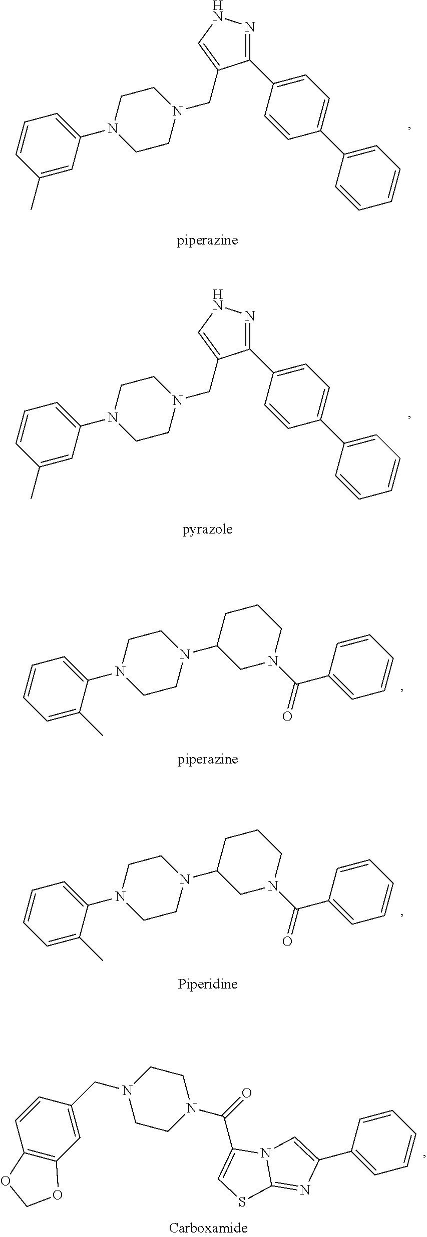 [SMILES: Cc1cccc(N2CCN(Cc3c[nH]nc3-c3ccc(-c4ccccc4)cc3)CC2)c1 ; Cc1cccc(N2CCN(Cc3c[nH]nc3-c3ccc(-c4ccccc4)cc3)CC2)c1 ; Cc1ccccc1N1CCN(C2CCCN(C(=O)c3ccccc3)C2)CC1 ; Cc1ccccc1N1CCN(C2CCCN(C(=O)c3ccccc3)C2)CC1 ; O=C(c1csc2nc(-c3ccccc3)cn12)N1CCN(Cc2ccc3c(c2)OCO3)CC1]

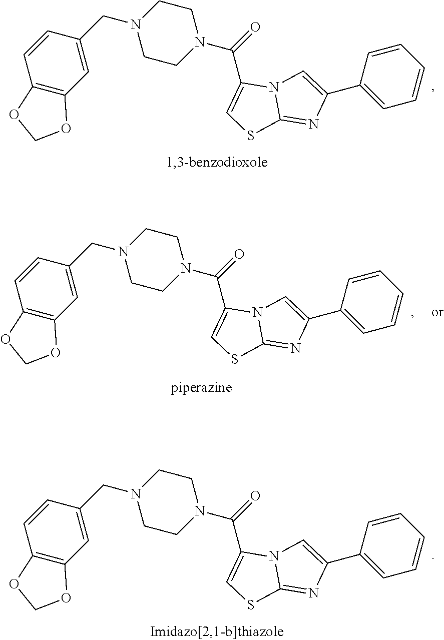 [SMILES: O=C(c1csc2nc(-c3ccccc3)cn12)N1CCN(Cc2ccc3c(c2)OCO3)CC1 ; O=C(c1csc2nc(-c3ccccc3)cn12)N1CCN(Cc2ccc3c(c2)OCO3)CC1 ; O=C(c1csc2nc(-c3ccccc3)cn12)N1CCN(Cc2ccc3c(c2)OCO3)CC1]

### A. Antagonists

Described herein are novel ZIP12 antagonists. ZIP12 antagonists can antagonize or otherwise inhibit a ZIP12 protein, for example, an astrocytic ZIP12 responsible for clearing Zn²⁺ from the synaptic cleft of a synapse. Inhibition with antagonists described herein can increase [Zn²⁺] in the synaptic cleft by reducing its ZIP12-mediated clearance from the cleft.

As described herein, a novel ZIP12 antagonist can be one of:

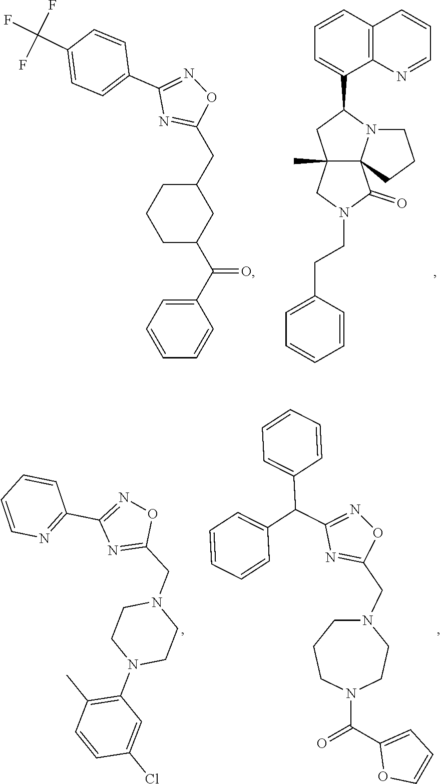

or a pharmaceutically-acceptable salt or prodrug thereof.

In examples, a ZIP12 antagonist of the present disclosure is a compound having the following structure:

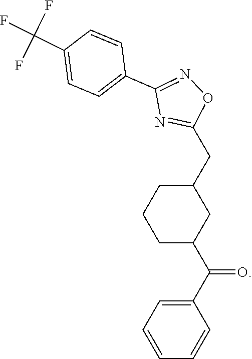 [SMILES: O=C(c1ccccc1)C1CCCC(Cc2nc(-c3ccc(C(F)(F)F)cc3)no2)C1]

In examples, a ZIP12 antagonist of the present disclosure is a compound having the following structure:

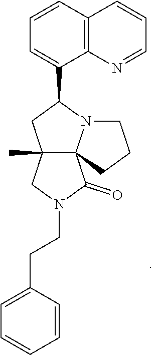 [SMILES: C[C@@]12C[C@@H](c3cccc4cccnc34)N3CCC[C@]31C(=O)N(CCc1ccccc1)C2]

In examples, a ZIP12 antagonist of the present disclosure is a compound having the following structure:

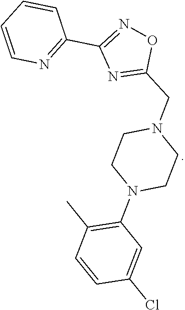 [SMILES: Cc1ccc(Cl)cc1N1CCN(Cc2nc(-c3ccccn3)no2)CC1]

In examples, a ZIP12 antagonist of the present disclosure is a compound having the following structure:

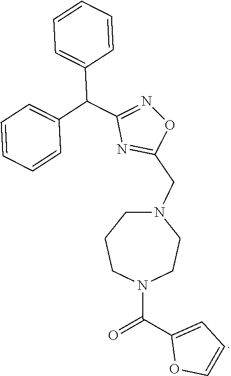 [SMILES: O=C(c1ccco1)N1CCCN(Cc2nc(C(c3ccccc3)c3ccccc3)no2)CC1]

### B. Agonists

Described herein are ZIP12 agonists. ZIP12 agonists can simulate a ZIP12 protein, for example, an astrocytic ZIP12 responsible for clearing Zn²⁺ from the synaptic cleft of a synapse. ZIP12 stimulation with agonists described herein can increase [Zn²⁺] in the synaptic cleft by increasing its ZIP12-mediated clearance from the cleft.

In examples, a ZIP12 agonist comprises a compound of formula (I):

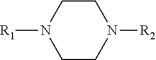

In examples, R₁ and R₂ are an optionally substituted aromatic ring, an optimally heteroaromatic ring or Ci-substituent, optimally having one or more heteroatoms, C(═O)R, C(═O)OR, or C(═O)NR.

In examples of the compound formula (I), the piperazine can be bonded to R1 or R2 accordingly:

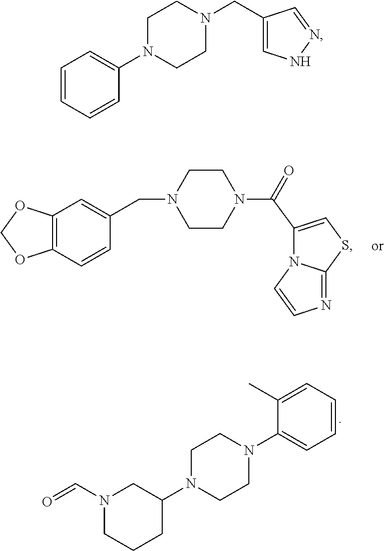 [SMILES: c1ccc(N2CCN(Cc3cn[nH]c3)CC2)cc1 ; O=C(c1csc2nccn12)N1CCN(Cc2ccc3c(c2)OCO3)CC1 ; Cc1ccccc1N1CCN(C2CCCN(C=O)C2)CC1]

In examples, a ZIP12 agonist comprises one or more of the following compounds of formula (I) (or pharmaceutically-acceptable salt or prodrug thereof):

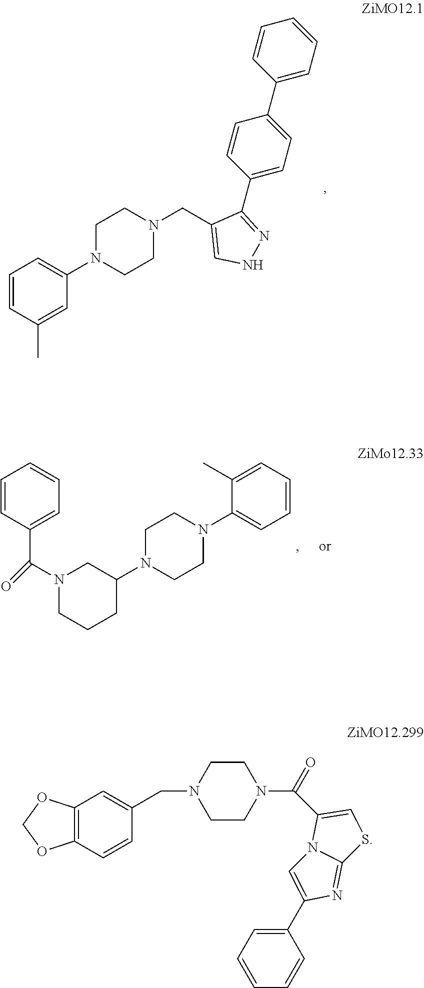 [SMILES: Cc1cccc(N2CCN(Cc3c[nH]nc3-c3ccc(-c4ccccc4)cc3)CC2)c1 ; Cc1ccccc1N1CCN(C2CCCN(C(=O)c3ccccc3)C2)CC1 ; O=C(c1csc2nc(-c3ccccc3)cn12)N1CCN(Cc2ccc3c(c2)OCO3)CC1]

### II. METHODS OF USE AND TREATMENT

Described herein are methods of modulating synaptic zinc, as well as methods of treatment.

In examples, described herein is a method of increasing zinc in the synaptic cleft, comprising administering one or more ZIP12 antagonists described herein, or a pharmaceutically-acceptable salt thereof, to a subject in need thereof.

In examples, the subject in need thereof has, or is suspected of having, dysregulated ZIP12 expression.

In examples, the one or more ZIP12 antagonists are administered in an effective amount in increase Zn²⁺ concentration in a synaptic cleft of a subject.

In examples, the one or more ZIP12 antagonists are administered at a concentration of about 1 μM to about 50 μM.

In examples, the one or more ZIP12 antagonists are administered at a concentration of 10 μM.

In examples, subject in thereof has a neuropsychiatric disorder caused by dysregulated ZIP12 expression, a neurodegenerative disorder caused by dysregulated ZIP12 expression, a neurodevelopmental disorder caused by dysregulated ZIP12 expression, a cancer caused by dysregulated ZIP12 expression, or inflammation caused by dysregulated ZIP12 expression.

In examples, the one or more ZIP12 antagonists are administered in an effective amount to alleviate one or more symptoms of the neurodegenerative disorder caused by dysregulated ZIP12 expression, the neurodevelopmental disorder caused by dysregulated ZIP12 expression, the cancer caused by dysregulated ZIP12 expression, or the inflammation caused by dysregulated ZIP12 expression.

In examples, the neuropsychiatric disorder caused by dysregulated ZIP12 expression is schizophrenia.

In examples, the neurodegenerative disorder caused by dysregulated ZIP12 expression is Alzheimer's Disease (AD) or Parkinson's Disease (PD).

In examples, the neurodevelopmental disorder caused by dysregulated ZIP12 expression is an autism spectrum disorder (ASD).

In examples, a cancer caused by ZIP12 dysregulation can be a breast cancer.

In examples, the one or more ZIP12 antagonists are delivered to the central nervous system of the subject.

In examples, the one or more ZIP12 antagonists are administered intracerebrally or intrathecally.

In examples, described herein is a method of decreasing zinc in the synaptic cleft, comprising administering one or more ZIP12 agonists described herein, or a pharmaceutically-acceptable salt thereof, to a subject in need thereof.

In examples, the subject in need thereof has, or is suspected of having, dysregulated ZIP12 expression.

In examples, the one or more ZIP12 agonists are administered in an effective amount in decrease Zn²⁺ concentration in a synaptic cleft of a subject.

In examples, methods as described herein can increase cytokine production in a sample or a subject having lower-than-normal cytokine production on account of low Zn²⁺ levels, for examples by increasing IL-2 production.

In examples, a method can comprise a negative control. In examples, the negative control can be ZiMo12.9 (N-{1-[5-methyl-1-(2-methylphenyl)-1H-pyrazol-4-yl]ethyl}cyclopropanecarboxamide; CC1=C(C═NN1C1=C(C═CC═C1)C)C(C)NC(═O)C1CC1).

At least the following evidence roles of compounds and compositions described herein in the context of methods described herein, all of which are incorporated by reference as if fully set forth herein:
Davis D N, Strong M D, Chambers E, Hart M D, Bettaieb A, Clarke S L, Smith B J, Stoecker B J, Lucas E A, Lin D, Chowanadisai W. A role for zinc transporter gene SLC39A12 in the nervous system and beyond. Gene. 2021 Oct. 5; 799:145824. doi: 10.1016/j.gene.2021.145824. Epub 2021 Jul. 9. PMID: 34252531; PMCID: PMC8318780;
Dean B, Hopper S, Scarr E. Changes in levels of the zinc transporter SLC39A12 in Brodmann's area 44: Effects of sex, suicide, CNS pH and schizophrenia. J Psychiatr Res. 2024 September; 177:177-184. doi: 10.1016/j.jpsychires.2024.07.017. Epub 2024 Jul. 14. PMID: 39024742;
Scarr E, Udawela M, Greenough M A, Neo J, Suk Seo M, Money T T, Upadhyay A, Bush A I, Everall I P, Thomas E A, Dean B. Increased cortical expression of the zinc transporter SLC39A12 suggests a breakdown in zinc cellular homeostasis as part of the pathophysiology of schizophrenia. NPJ Schizophr. 2016 Mar. 9; 2:16002. doi: 10.1038/npjschz.2016.2. PMID: 27336053; PMCID: PMC4898896;
Manning A, Mendelson B Z, Bender P T R, Bainer K, Ruby R, Shifflett V R, Dariano D F 3rd, Webb B A, Geldenhuys W J, Anderson C T. The Astrocytic Zinc Transporter ZIP12 Is a Synaptic Protein That Contributes to Synaptic Zinc Levels in the Mouse Auditory Cortex. J Neurosci. 2025 Mar. 26; 45(13):e2067242025. doi: 10.1523/JNEUROSCI.2067-24.2025. PMID: 39809542; PMCID: PMC11949477;
Barman S K, Nesarajah A N, Zaman M S, Malladi C S, Mahns D A, Wu M J. Distinctive expression and cellular localisation of zinc homeostasis-related proteins in breast and prostate cancer cells. J Trace Elem Med Biol. 2024 December; 86:127500. doi: 10.1016/j.jtemb.2024.127500. Epub 2024 Jul. 22. PMID: 39047373;
Qu Y Y, Guo R Y, Luo M L, Zhou Q. Pan-Cancer Analysis of the Solute Carrier Family 39 Genes in Relation to Oncogenic, Immune Infiltrating, and Therapeutic Targets. Front Genet. 2021 Dec. 2; 12:757582. doi: 10.3389/fgene.2021.757582. PMID: 34925450; PMCID: PMC8675640; and
Daaboul D, Rosenkranz E, Uciechowski P, Rink L. Repletion of zinc in zinc-deficient cells strongly up-regulates IL-1β-induced IL-2 production in T-cells. Metallomics. 2012 October; 4(10):1088-97. doi: 10.1039/c2mt20118f. Epub 2012 Sep. 14. PMID: 22983538.

### III. KITS AND PACKAGING

Described herein are kits for modulating zinc in the synaptic cleft, as well as therapeutic kits. In certain aspects, kits may also contain nucleic acids and/or vectors encoding a functional ZIP12 protein (for example, a vector having a polynucleotide encoding NCBI RefSeq NM_001145195.2 relating to NCBI GeneID 221074, the sequences of which are incorporated by reference as if fully set forth herein). Kits can also comprise any compounds described herein.

Vectors according to kits here can be, as described, polynucleotides encoding a functional ZIP12 protein that can be transfected, transduced, or otherwise introduced into a eukaryotic cell as described herein by methods known in the art to introduce polynucleotides into a cell through its lipid bilayer. Vectors described herein can be viral or non-viral vectors. In examples, a vector is a lentiviral vector that can be packaged into a lentivirus for viral transduction into a cell, such as a HEK cell or mammalian primary astrocyte or other mammalian CNS cell, for example. In certain aspects, a vector may be optimized for transfection in a CNS cell, for example, by operably linking the ZIP12 encoding sequence to an astrocytic promotor, such as a glial fibrillary acidic protein (GFAP) for example, or other codon-optimization.

In another embodiment, this kit comprises a (optionally sterile) solvent suitable for dissolving or suspending a composition of the presently disclosed subject matter prior to use.

In embodiments, a kit comprises one or more compounds, and instructions for use, for example, in modulating zinc in the synaptic cleft. A kit can comprise one or more ZIP12 antagonists according to the present disclosure. A kit can comprise one or more ZIP12 agonists according to the present disclosure.

Described herein are containers for storage and/or use, and instructions for use. The kit can be a package which houses a container which contains compounds of the disclosure or formulations of the disclosure and also houses instructions for administering the compounds or formulations for use. The disclosure further relates to a commercial package comprising compounds of the disclosure or formulations of the disclosure together with instructions for simultaneous, separate or sequential use. In particular a label may include amount, frequency, and method of use.

As used herein, an “instructional material” includes a publication, a recording, a diagram, or any other medium of expression which can be used to communicate the usefulness of the components thereof of the presently disclosed subject matter in the kit for synaptic zinc modulation and therapeutic administration. The instructional material of the kit of the presently disclosed subject matter may, for example, be affixed to a container which contains a composition of the presently disclosed subject matter or be shipped together with a container which contains the composition. Alternatively, the instructional material may be shipped separately from the container with the intention that the instructional material and the composition be used cooperatively by the recipient

The disclosure also relates to articles of manufacture and kits containing materials useful for using any one or more compounds disclosed herein. An article of manufacture may comprise a container with a label. Examples of suitable containers include bottles, vials, and test tubes, or a delivery device, which may be formed from a variety of materials including glass and plastic. A container holds compounds of the disclosure or formulations of the disclosure which are effective for modulating synaptic zinc and/or treating a disease disclosed herein. The label on the container indicates that the compounds of the disclosure or formulations of the disclosure are used for applications disclosed herein and may also indicate directions for use.

The disclosure also contemplates kits comprising one or more compounds for compositions of the disclosure. In aspects of the disclosure, a kit of the disclosure comprises a container described herein. In particular aspects, a kit of the disclosure comprises a container described herein and a second container comprising a buffer. A kit may additionally include other materials desirable from a commercial and user standpoint, including, without limitation, buffers, diluents, filters, needles, syringes, and package inserts with instructions for performing any methods disclosed herein.

The compositions (i.e., those comprising, consisting essentially of, or consisting of enzymes described herein) can be utilized in the preparation of a kit. In some embodiments, kits are provided for carrying out any of the methods described herein. The kits of this disclosure may comprise a carrier container being compartmentalized to receive in close confinement one or more containers such as vials, tubes, and the like, each of the containers comprising one of the separate elements to be used in the methods.

In some instances, one of the containers may comprise a composition as described in this disclosure that is, or can be, detectably labeled. The kit may also have containers containing buffer(s) and/or a container comprising a reporter-means, such as a biotin-binding protein, such as avidin or streptavidin, bound to a reporter molecule, such as an enzymatic or fluorescent label. In some embodiments, the kit comprises separate containers containing compositions described herein and a detectable label. In some aspects, the vehicle for kits to be used to create compositions described herein comprising a compound of the present disclosure is DMSO.

### A. Research Kits

In an embodiment, a kit for modulating synaptic zinc is described comprising one or more compounds described herein. In certain aspects, a kit can comprise one or more compounds, and Instructions for use. A kit can further comprise one or more compounds described herein.

A composition or antibody thereof as described in this disclosure for use in modulating synaptic zinc may be delivered in a package to research or clinical labs. Typically, the packaging comprises paper (cardboard) or plastic. In some embodiments, the kit further comprises instructions for use.

In examples, a kit can comprise a negative control. In examples, the negative control can be ZiMo12.9 (N-{1-[5-methyl-1-(2-methylphenyl)-1H-pyrazol-4-yl]ethyl}cyclopropanecarboxamide; CC1=C(C═NN1C1=C(C═CC═C1)C)C(C)NC(═O)C1CC1).

### B. Clinical Kits

In some embodiments, a kit comprises unit dose forms of a composition or components of compositions described herein (i.e., those comprising one or more compounds described herein).

In one embodiment, the kit comprises a composition as described herein in a defined, an effective dose in a single unit dosage form or as separate unit doses. The dose and form of the unit dose can be any doses or forms as described herein.

In certain embodiments, kits containing one or more containers of a formulation described in this disclosure are included

In an embodiment, a kit can comprise one or more compounds described herein, a polynucleotide described herein, a vector described herein, and/or a pharmaceutical composition described herein; and instructions for use.

In certain aspects, the one or more compounds described herein, polynucleotide described herein, vector described herein, and/or a pharmaceutical composition described herein can be provided in a dosage unit form.

### IV. PHARMACEUTICAL FORMULATIONS AND ROUTES OF ADMINISTRATION

Embodiments of the present disclosure include any one or more compounds as identified herein and formulated with one or more pharmaceutically acceptable excipients, diluents, carriers and/or adjuvants. In addition, embodiments of the present disclosure include one or more compounds formulated with one or more pharmaceutically acceptable auxiliary substances. In particular, any one or more compounds of the present disclosure can be formulated with one or more pharmaceutically acceptable excipients, diluents, carriers, and/or adjuvants to provide an embodiment of a composition of the present disclosure. While reference is made below to “a compound”, any one or more compounds according to the present disclosure can be utilized for pharmaceutical preparations according to the present disclosure. In examples, only one compound is used for a given pharmaceutical preparation.

A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In an embodiment of the present disclosure, any one or more compounds can be administered to the subject using any means capable of resulting in the desired effect. Thus, one or more compounds can be incorporated into a variety of formulations for therapeutic administration. For example, one or more compounds can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, a compound may be administered in the form of its pharmaceutically acceptable salts, or a subject active composition may be used alone or in appropriate association, as well as in combination, with other pharmaceutically active compounds. The following methods and excipients are merely exemplary and are in no way limiting.

For oral preparations, a compound can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Embodiments of a compound can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Embodiments of the compound can be utilized in aerosol formulation to be administered via inhalation. Embodiments of a compound can be formulated into pressurized acceptable propellants such as dichlorodifluoromethane, propane, nitrogen and the like.

Furthermore, embodiments of a compound can be made into suppositories by mixing with a variety of bases such as emulsifying bases or water-soluble bases. Embodiments of a compound can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compositions. Similarly, unit dosage forms for injection or intravenous administration may comprise a compound in a composition as a solution in sterile water, normal saline or another pharmaceutically acceptable carrier.

Embodiments of a compound can be formulated in an injectable composition in accordance with the disclosure. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles in accordance with the present disclosure.

In an embodiment, a compound can be formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of a compound can be accomplished using any of a variety of refillable, pump systems. Pumps provide consistent, controlled release over time. In some embodiments, a compound can be in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, an active agent (a compound of the present disclosure) can be delivered using an implantable drug delivery system, e.g., a system that is programmable to provide for administration of the agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present disclosure is the Synchromed infusion pump (Medtronic).

Suitable excipient vehicles for a compound are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pennsylvania, 17th edition, 1985. The composition or formulation to be administered will, in any event, contain a quantity of a compound adequate to achieve the desired state in the subject being treated.

Compositions of the present disclosure can include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the pharmaceutical composition of the present disclosure (as well as combination compositions) can be delivered in a controlled release system. For example, a compound may be administered using intravenous infusion, an implantable osmotic pump, a transdermal patch, liposomes, or other modes of administration. In one embodiment, a pump may be used (Sefton (1987). CRC Crit. Ref. Biomed. Eng. 14:201; Buchwald et al. (1980). Surgery 88:507; Saudek et al. (1989). N. Engl. J. Med. 321:574). In another embodiment, polymeric materials are used. In yet another embodiment a controlled release system is placed in proximity of the therapeutic target thus requiring only a fraction of the systemic dose. In yet another embodiment, a controlled release system is placed in proximity of the therapeutic target, thus requiring only a fraction of the systemic. Other controlled release systems are discussed in the review by Langer (1990). Science 249:1527-1533.

In another embodiment, the compositions of the present disclosure (as well as combination compositions separately or together) include those formed by impregnation of a compound described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

### A. Dosages

Embodiments of a compound can be administered to a subject in one or more doses. Those of skill will readily appreciate that dose levels can vary as a function of the specific compound administered, the severity of the symptoms and the susceptibility of the subject to side effects. Preferred dosages for a given compound are readily determinable by those of skill in the art by a variety of means.

In an embodiment, multiple doses of a compound are administered. The frequency of administration of a compound can vary depending on any of a variety of factors, e.g., severity of the symptoms, and the like. For example, in an embodiment, a compound can be administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). As discussed above, in an embodiment, a compound is administered continuously.

The duration of administration of a compound analogue, e.g., the period of time over which a compound is administered, can vary, depending on any of a variety of factors, e.g., patient response, etc. For example, a compound in combination or separately, can be administered over a period of time of about one day to one week, about two weeks to four weeks, about one month to two months, about two months to four months, about four months to six months, about six months to eight months, about eight months to 1 year, about 1 year to 2 years, or about 2 years to 4 years, or more.

In examples, a dosage for ZIP12 antagonists and agonists described herein can be a concentration of about 1 μM to about 50 μM. In examples, a dosage for ZIP12 antagonists and agonists described herein can be a concentration of about of 10 μM.

### B. Routes of Administration

Embodiments of the present disclosure provide methods and compositions for the administration of the active agent (e.g., a compound) to a subject (e.g., a human) using any available method and route suitable for drug delivery, including in vivo and ex vivo methods, as well as systemic and localized routes of administration.

Routes of administration include intranasal, intramuscular, intratracheal, subcutaneous, intradermal, topical application, intravenous, rectal, nasal, oral, and other enteral and parenteral routes of administration. Routes of administration may be combined, if desired, or adjusted depending upon the agent and/or the desired effect. An active agent (e.g., a compound) can be administered in a single dose or in multiple doses. In certain aspects, the delivery may be intracerebral, intraventricular, or intrathecal.

Embodiments of a compound can be administered to a subject using available conventional methods and routes suitable for delivery of conventional drugs, including systemic or localized routes. In general, routes of administration contemplated by the disclosure include, but are not limited to, enteral, parenteral, or inhalational routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of a compound. Where systemic delivery is desired, administration typically involves invasive or systemically absorbed topical or mucosal administration of pharmaceutical preparations.

Methods of administration of a compound through the skin or mucosa include, but are not limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

Other features, objects, and advantages of the present disclosure are apparent in the description that follows. It should be understood, however, that the description, while exemplifying certain embodiments of the present disclosure, is given by way of illustration only, not limitation. Various changes and modifications within the scope of the disclosure will become apparent to those skilled in the art from the detailed description.

### V. EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

### Example 1: The Astrocytic Zinc Transporter ZIP12 is a Synaptic Protein that Contributes to Synaptic Zinc Levels in the Mouse Auditory Cortex

Synaptically released zinc is a neuronal signaling system that arises from the actions of the presynaptic vesicular zinc transporter protein zinc transporter 3 (ZnT3). Mechanisms that regulate the actions of zinc at synapses are of great importance for many aspects of synaptic signaling in the brain. The astrocytic zinc transporter protein ZIP12 was identified as a candidate mechanism that contributes to zinc clearance at cortical synapses. Small-molecule compounds that antagonize the function of ZIP12 were identified in heterologous expression systems, and one of these compounds, ZIP12 modulator 8, was used to increase the concentration of ZnT3-dependent zinc at synapses in the brain of male and female mice to inhibit the activity of neuronal AMPA and NMDA glutamate receptors. These results identify a cellular mechanism and provide a pharmacological toolbox to target the molecular machinery that supports the actions of synaptic zinc in the brain.

Synaptic zinc is loaded into presynaptic glutamatergic vesicles by the protein zinc transporter 3 (ZnT3), where it is co-released with glutamate during synaptic transmission. Evidence from clinical studies in humans shows that alterations in the expression of the neuronal zinc transporter protein ZnT3 and the astrocytic zinc transporter protein ZIP12 are associated with schizophrenia, suggesting that dysregulation of these brain-specific zinc transporter proteins may contribute altered synaptic signaling in the brain. The results show that ZIP12 protein is expressed by astrocytes at synapses in the brain. Pharmacological agents that inhibit ZIP12 were identified and used to modulate zinc levels in the brain. This research advances understandings of the roles of synaptic zinc in health and disease.

### a) Introduction

Zinc is enriched in the neocortex with ~20% of total cortical zinc found in glutamatergic synaptic vesicles (Cole et al., 1999). This pool of zinc is termed “synaptic zinc” because it is loaded into presynaptic glutamatergic vesicles by the protein zinc transporter 3 (ZnT3) and released into the synaptic cleft along with glutamate during synaptic transmission (Cole et al., 1999; Frederickson et al., 2005; McAllister and Dyck, 2017a). There are 11 members of the zinc transporter family of proteins (ZnT, Slc30a1-11) that reduce cytoplasmic zinc by transporting it out of cells or into intracellular compartments (including synaptic vesicles) and 14 members of the Zrt, Irt-like family of proteins (ZIP, Slc39a1-14) that increase cytoplasmic zinc by transporting it into the cytoplasm from extracellular domains (ECD) and endosomal compartments (Kambe et al., 2015; Styrpejko and Cuajungco, 2021). Different tissues have different expression levels of ZnT and ZIP proteins, and some, such as ZnT3 and ZIP12, are selectively and highly expressed in the brain (Palmiter et al., 1996; Seve et al., 2004; Chowanadisai et al., 2013) with high ZnT3 expression in a large subset of neurons (Zeisel et al., 2015; Chen et al., 2019) and high ZIP12 expression in astrocytes (Zhang et al., 2014a; Nishikawa et al., 2017; Boisvert et al., 2018), a major class of glia. Since astrocytes are an integral component of excitatory synapses (Perea et al., 2009) crucial for controlling the dynamics of glutamate reuptake after its release (Rose et al., 2018), they are well positioned to regulate other aspects of excitatory transmission, such as synaptic zinc levels.

During neurotransmission, free zinc levels in the synaptic cleft rise from nanomolar to tens of micromolar levels over millisecond timescales, returning to basal nanomolar levels within a few hundred milliseconds (Vogt et al., 2000; Vergnano et al., 2014; Morabito et al., 2022; Bender et al., 2023). This suggests the presence of a powerful system for the rapid removal of free zinc following its release, but the molecular components of this system remain unknown. ZIP12 (Slc39a12) has a very high affinity for zinc with an apparent Km value of ~6 nM (Chowanadisai et al., 2013). Crucially, this high affinity of ZIP12 for zinc is appropriate to set the low nanomolar levels of free zinc near synaptic clefts (Vergnano et al., 2014; Anderson et al., 2015). Mounting evidence from clinical studies with humans shows that changes in the expression of ZnT3 and ZIP12 are associated with schizophrenia—a major neuropsychiatric disorder that affects ~1% of people worldwide (Insel, 2010). By analyzing postmortem brain tissue samples from people withschizophrenia compared with age-matched controls, recent studies found that schizophrenia is associated with reduced cortical ZNT3 mRNA and protein expression (Perez-Becerril et al., 2016) and with increased ZIP12 mRNA expression (Scarr et al., 2016; Dean et al., 2024). This indicates that ZnT3 and ZIP12 may regulate zinc signaling in people and suggests that their dysregulation may contribute to the etiology of this disorder. To date, efforts to pharmacologically affect synaptic zinc signaling using the metal chaperones clioquinol and PBT2 have had beneficial effects on the cognitive deficits observed in ZnT3 knock-out (KO) mice (Adlard et al., 2015) and aging wildtype (WT) mice (Adlard et al., 2014); however, these approaches do not exclusively affect synaptic zinc signaling, so there is a crucial need to develop more specific tools.

Here, it is shown that ZIP12 is expressed at cortical synapses, small-molecule compounds that specifically target the function of ZIP12 in heterologous expression systems (ZIP modulators; ZiMos) are identified, and one of these compounds, ZIP12 modulator 8 (ZiMo12.8), is used to increase the inhibitory effects of synaptically released zinc on neuronal AMPA and NMDA glutamate receptors in acute brain slices of the mouse auditory cortex (ACx). Together, the results uncover mechanisms that shape synaptically released zinc and identify a pharmacological approach to target the molecular machinery important for the function of synaptic zinc at synapses in the brain.

### b) Materials and Methods

### Animal Handling

WT C57Bl/6 mice (Jackson Laboratory) and ZnT3 KO mice (Jackson Laboratory) aged Postnatal Day (P)32-P35 (for histology experiments) and aged P21-P35 (for electrophysiology experiments) were used in accordance with the animal welfare guidelines and regulations of West Virginia University, the US National Institutes of Health, and the Society for Neuroscience. All procedures were approved by the Institutional Animal Care and Use Committee of West Virginia University. Both male and female mice were used in these experiments. Experiments using ZnT3 KO mice were performed blind to their genotype.

### Perfusions

Male and female WT mice aged P35 were anesthetized and perfused transcardially using carbogenated artificial cerebral spinal fluid (ACSF; in mM: 130 NaCl, 3 KCl, 2.4 CaCl2, 1.3 MgCl2, 20 NaHCO₃, 3 HEPES, 10 D-glucose; saturated with 95% O2/5% CO2 (vol/vol), ~300 mOsm), pH 7.25-7.35, followed by 4% paraformaldehyde (PFA) with 0.0028% glutaraldehyde in phosphate-buffered saline (PBS). Brains were immediately removed and placed into PFA overnight. Brains were then cryopreserved in 30% sucrose in PBS and sectioned coronally or sagittally at 25 μm on a microtome (Thermo Fisher Scientific Sliding Microtome Microm HM450) with a BFS-40MPA Freezing Stage (Physitemp Instruments). Sections were stored free-floating in 0.01% sodium azide in PBS at 4° C.

### Immunohistochemistry

Sections were first blocked for 1 h at room temperature in blocking buffer [1% bovine serum albumin (BSA), 10% fetal bovine serum, 1% Triton X-100 in PBS]. Sections were then incubated for 48 h at 4° C. with primary antibody (chicken anti-GFP, 1:1,000, Abcam, 13970; guinea pig anti-bassoon, 1:200, Synaptic Systems, 141 004; mouse IgG1 anti-ZnT3, 1:1,000, Synaptic Systems, 197 011; chicken anti-EAAT1, 1:1,000, Synaptic Systems, 250 116; mouse IgG1 anti-MAP2, 1:1,000, Synaptic Systems, 188 011; rabbit anti-ZIP12, 1:1,000, Sigma-Aldrich, AV44127) in blocking buffer and washed three times for 10 min in PBS. Sections incubated with secondary antibody (goat anti-chicken Alexa Fluor 488, Jackson ImmunoResearch Laboratories, 103-545-1555; goat anti-guinea pig Alexa Fluor 555, Invitrogen, A-21435; goat anti-mouse IgG1 Alexa Fluor 555, Invitrogen, A-21127; goat anti-rabbit Alexa Fluor 594, Jackson ImmunoResearch Laboratories, 111-585-144; goat anti-mouse IgG1 Alexa Fluor 594, Jackson ImmunoResearch Laboratories, 115-587-158; goat anti-rabbit Alexa Fluor Plus 647, Invitrogen, A32733TR; goat antirabbit Alexa Fluor 750, Invitrogen, A-21039) in blocking buffer (1:500) for 2 h at room temperature, washed three times in PBS for 10 min, washed two times in 0.1 M phosphate buffer for 5 min, and mounted to glass slides with ProLong Glass Antifade Mountant (Invitrogen) and covered with a No. 1.5 glass coverslip.

### Fluorescent Imaging and Analysis

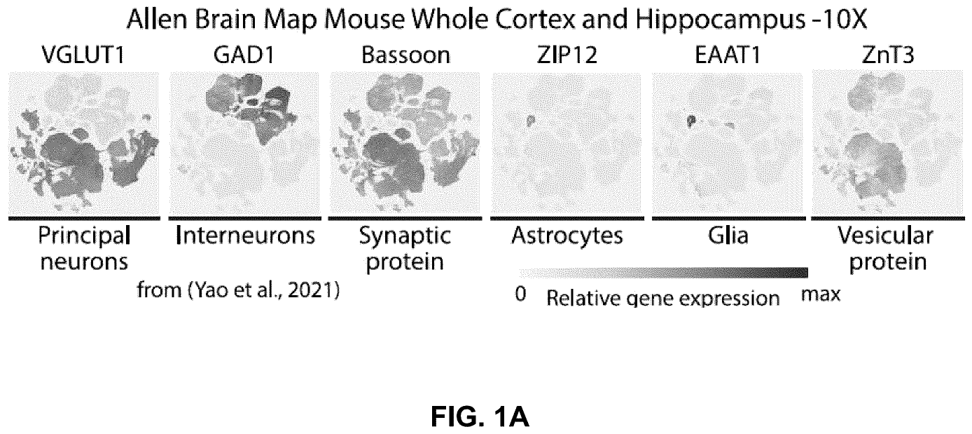

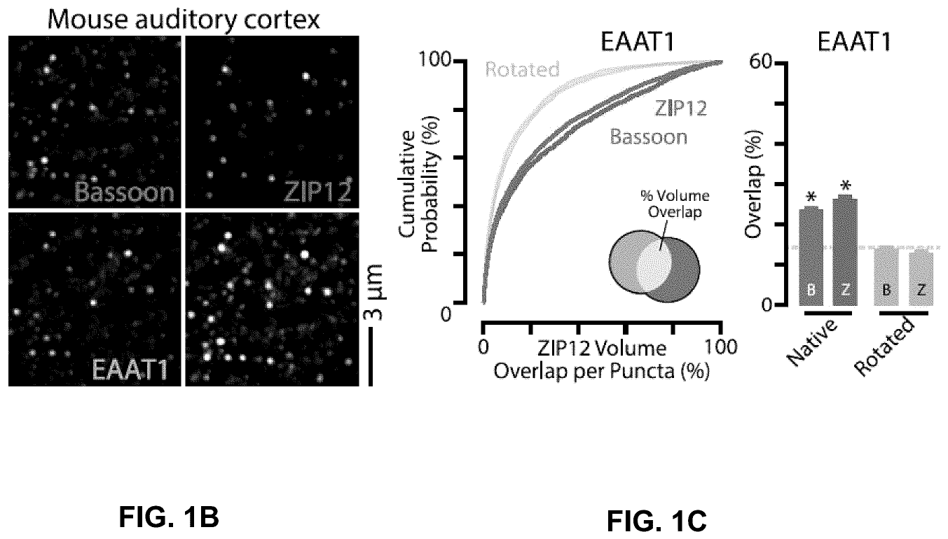

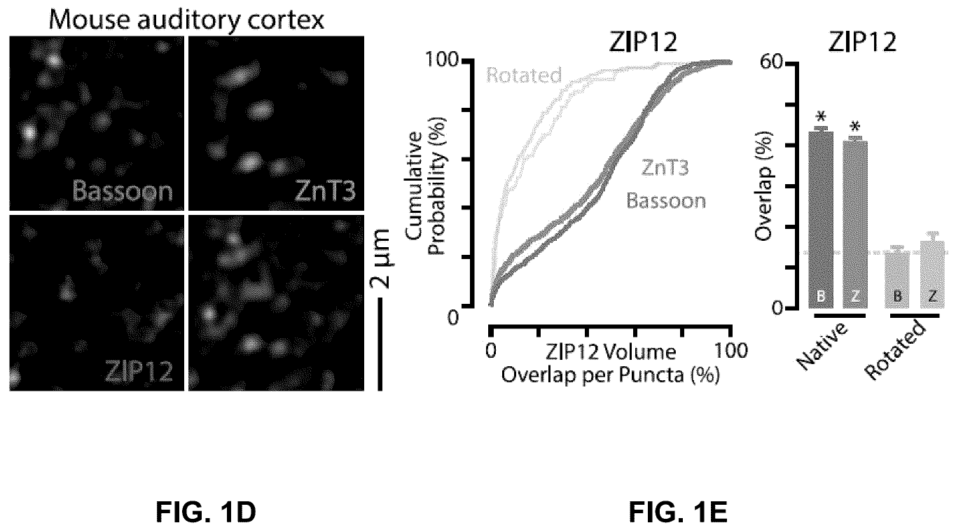

The 20×20 μm(1,024×1,024 pixel) confocal Z-stacks (0.2 μmZ-step) were obtained 0 using Leica Stellaris 8 with a 100× oil immersion objective. Z-stacks were analyzed using Fiji (Schindelin et al., 2012). A Gaussian blur (sigma of 0.04 μm) was applied to each image to filter out noise. To determine puncta distance and puncta overlap, images were analyzed through the Fiji plugin DiAna. Puncta were determined with an XY radius of 3.0 (0.06 μm) and Z radius of 2.0 (0.04 μm) and noise of 15.0. Thresholding was set to the mean fluorescence intensity of the Z-stack+2×standard deviation of the mean fluorescence intensity and a minimum volume of 15 pixels. Puncta overlap was calculated as the percentage of each EAAT1 (FIG. 1C) or ZIP12 (FIG. 1E) punctum volume that overlapped with a bassoon, ZnT3, or ZIP12 punctum. Pairs of puncta with zero percentage overlap are excluded from these calculations. To determine the dendritic spine size, fluorescent images of GFP-expressing dendrites were obtained, and the head of each spine within each image was selected and thresholded. The area of each spine head was determined automatically using Fiji's measurement tool. The presence of a protein at each synapse was determined through a statistical measure of puncta fluorescence against the background fluorescence of a 1×1 μm area around the puncta. Expression was defined as the mean puncta fluorescence intensity being greater than the mean+2× standard deviation of the mean background fluorescence intensity. Fluorescence intensity was determined automatically using Fiji's measurement tool. Rotation analysis was performed by rotating one of the fluorescent channels in multicolor images 90° clockwise as indicated in the corresponding figure legend. This analysis can reveal spatial association between puncta if image rotation reduces the number of spatially overlapping pairs of puncta and the percentage of overlapping pixels shared by overlapping puncta; the number of pairs of puncta resulting from rotation analysis is listed in the corresponding figure legend.

### HEK293 Compound Screening

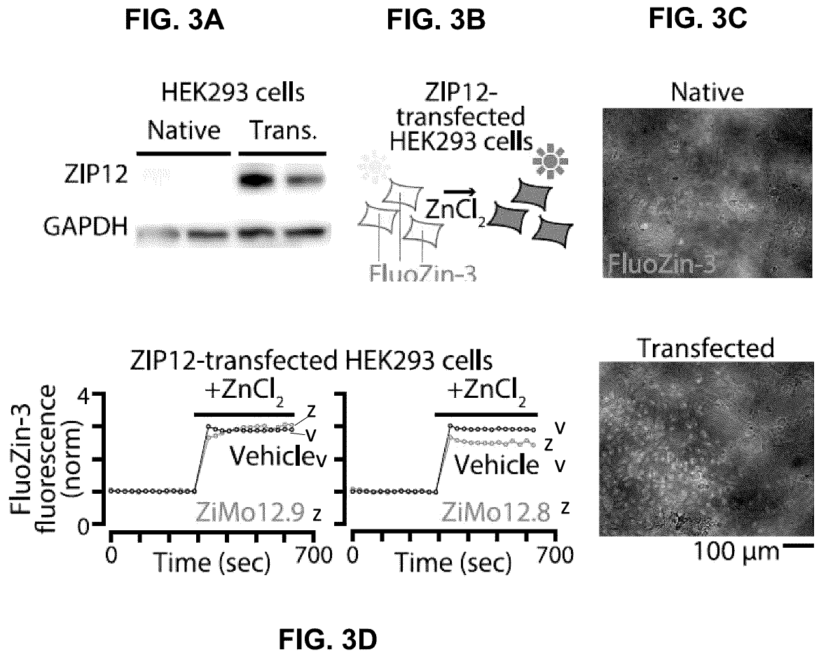

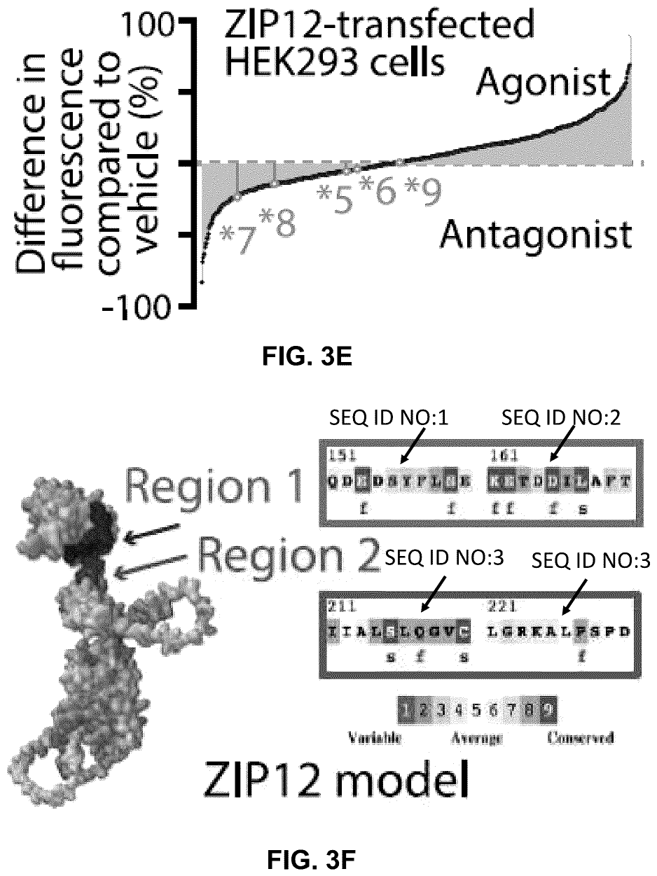

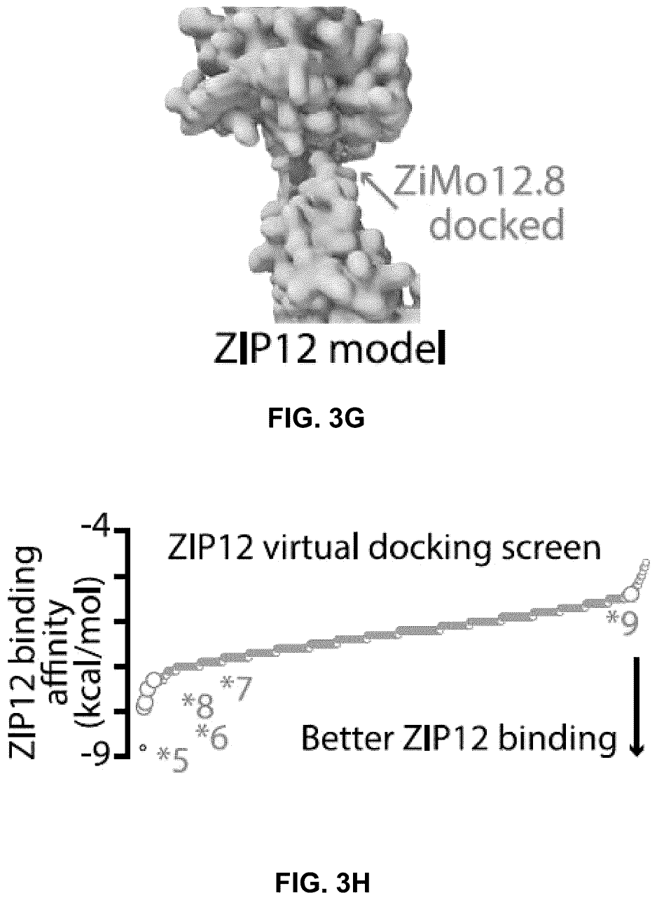

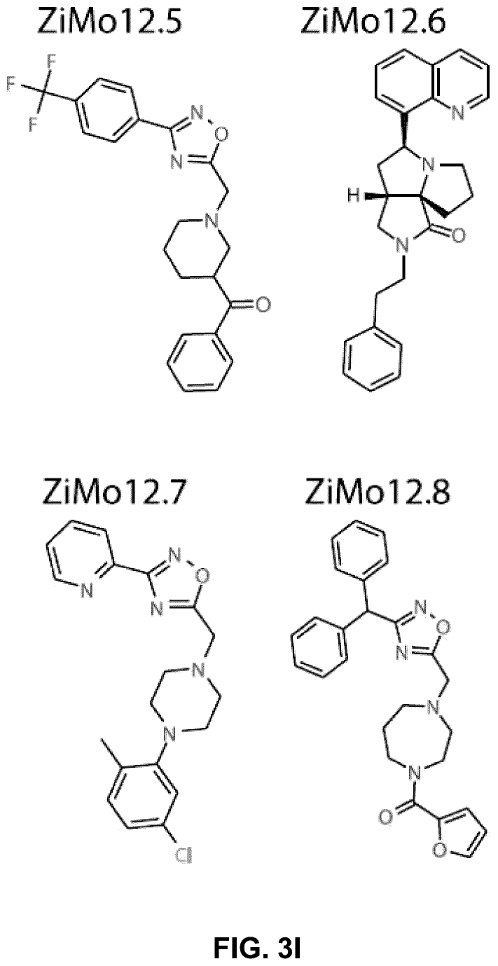

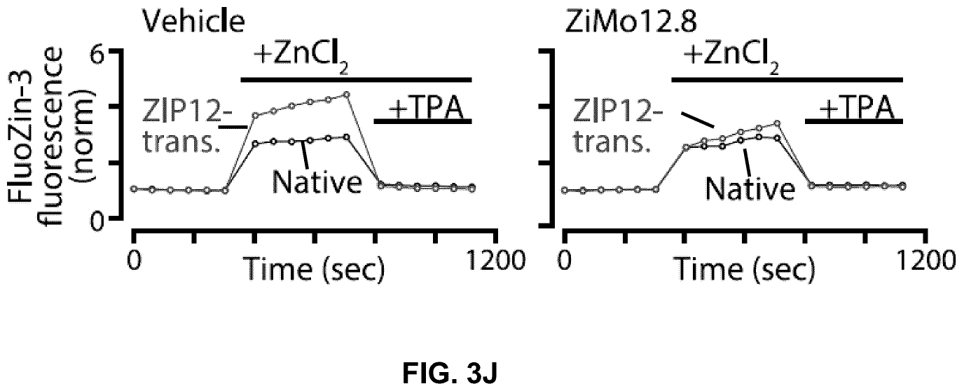

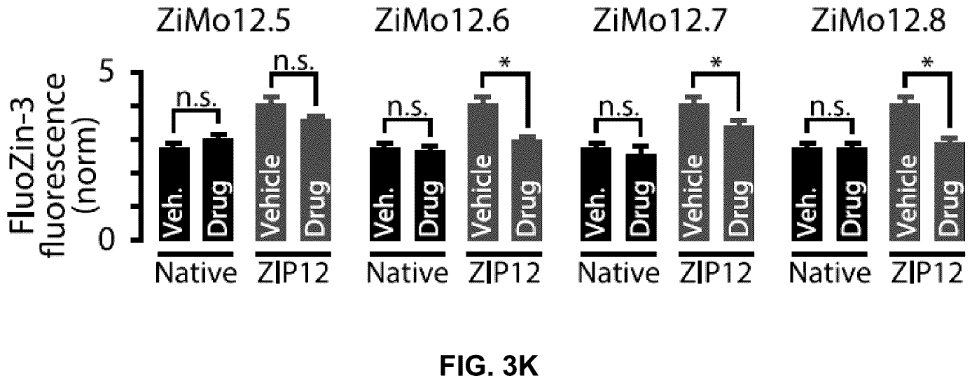

HEK293 cells (ATCC) were transfected with 0.67 mg of human Myc-DDK-ZIP12 cDNA (OriGene plasmid, RC227331) using FuGene HD (Qiagen). ZIP12 cDNA-transfected HEK293 cells were then placed in 60 wells of a 98-well plate. Cell culture media were vacuumed from wells and 60 μl of a 1 μM FluoZin-3, AM and 1 μM CellTracker Red CMPTX (Thermo Fisher Scientific) in clear serum-free media were added to each well. The cells were incubated for 30 min at 37° C. After incubation, the media were removed and replaced with clear HBSS. The wells were then rinsed, and each well was given either one compound (1 μM per well, 50 wells) or a dimethyl sulfoxide (DMSO) vehicle (10 wells). The well plate was immediately taken to a plate reader for fluorescent zinc uptake assay. The assay consisted of 300 s of a baseline measurement of FluoZin-3 fluorescence intensity without any ZnCl2 and then 300 s of measuring fluorescence intensity after the addition of 0.5 μM ZnCl2. The zinc-insensitive CellTracker Red CMPTX fluorescence was also measured throughout the assay as a control and used as a normalization for FluoZin-3 cell fluorescence. To normalize fluorescence data, a ratio was calculated by dividing the FluoZin-3 fluorescence intensity measurements by the CellTracker Red CMPTX fluorescence intensity measurements within the same well. This normalization accounted for variation in cell number between wells. The change in fluorescence from the baseline to the addition of ZnCl2 was then calculated for each compound and compared with the vehicle (FIG. 3D). The difference to the vehicle for each compound was then graphed and compared with every other compound, where a negative difference indicated an antagonist (FIG. 3E). For experiments shown in FIG. 3, J and K, the high-affinity, fast, cell-permeant zinc chelator TPA (20 μM; Huang et al., 2013) was added to the bath to reduce the zinc-dependent fluorescence of FluoZin-3 and verify that the fluorescence increases following ZnCl2 addition were not due to non zinc dependent sources such as autofluorescence signals that can increase over time due to metabolic stress (Surre et al., 2018) or due to phototoxicity from the excitation light used in fluorescent imaging (Icha et al., 2017).

### Virtual Compound Screening

The virtual screen was performed by docking Hit2Lead (ChemBridge) compounds into putative binding pockets of the ZIP12-ECD. The structure of ZIP12 was obtained from AlphaFold v4 (AF-Q5FWH7-F1-v4) and the dimerization interface identified by a structural and sequence alignment of this model with the ZIP4-ECD monomer (PDBID:4XA2). From the Hit2Lead compound list, 3-D .sdf files of each compound are generated by conversion of their SMILES to 2-D representations, and then 3-D representations using the MMF94+s forcefield in RDKit (rdkit.org). The subregion of the ZIP12 model was selected, and each ligand was virtually docked within the AutoDock Vina Extension available in the SAMSON software suite (Trott and Olson, 2010; Eberhardt et al., 2021). Using the DataWarrior structural-activity relationship (SAR) suite (openmolecules.org), analysis was performed by combining data from both previously screened and unscreened compounds to determine substructure similarities that consistently resulted in high inhibition of “in cell” tests (Sander et al., 2015).

### Western Blot

Cell culture samples were obtained by first rinsing cells within the six-well plate with cold PBS and then vacuumed. More PBS was added, and a cell scraper was used to free cells from the bottom of the well plate to be pipetted into microcentrifuge tubes. The cells were centrifuged for 5 min at 1,500 RPM, and the supernatant was discarded. Samples were then incubated with RIPA buffer (Sigma-Aldrich) and a protease inhibitor cocktail (1 mMAEBSF, 800 nM aprotinin, 50 μMbestatin, 15 μM×10-64, 20 μMleupeptin, 10 μMpepstatin A, 5 mMEDTA; Thermo Fisher Scientific) on ice for 30 min, centrifuged for 10 min at 12,000 RPM and 4° C., and the supernatant was saved. Using the Pierce BCA Protein Assay Kit (Thermo Fisher Scientific), the concentration of each sample was determined, and sample concentrations were then reduced to the lowest sample concentration using Milli-Q ultrapure water. Samples were then mixed 1:1 with 5% β-mercaptoethanol (Thermo Fisher Scientific) in 2× Laemmli sample buffer (Bio-Rad Laboratories) and boiled for 5 min at 100° C. Samples were separated on 4-20% Mini-PROTEAN TGX Gels (Bio-Rad Laboratories) and transferred

to 0.2 μm PVDF membrane (Bio-Rad Laboratories) using a 25 mM Tris, 192 mM glycine, 0.1% (w/v) SDS buffer (Bio-Rad Laboratories). The membrane was first blocked using 3% BSA (G-Biosciences) for 1 h at room temperature and then incubated with rabbit anti-ZIP12 primary antibody in 3% BSA (1:1,000, Sigma-Aldrich, AV44127) overnight at 4° C. The membrane was washed three times in PBS (Fisher Bioreagents) with 0.1% Tween 20 (T, Sigma-Aldrich) for 15 min. The membrane was then incubated with goat anti-rabbit secondary antibody conjugated to horse radish peroxidase (1:2,000, Bio-Rad Laboratories, 1721019) in PBST for 1 h at room temperature and washed three times for 15 min with PSBT. SuperSignal West Pico PLUS Stable Peroxide (Thermo Fisher Scientific) was mixed 1:1 with SuperSignal West Pico PLUS Luminol/Enhancer (Thermo Fisher Scientific) and placed gently over the membrane and imaged using an Amersham Imager 680 (GE Life Sciences). After imaging, the membrane was rinsed with PBST, incubated with rabbit anti-glyceraldehyde 3-phosphate dehydrogenase (GAPDH; 1:1,000, Bio-Rad Laboratories, VPA00187) in 3% BSA overnight at 4° C., and then washed three times with PBST for 15 min. The membrane was incubated with goat anti-rabbit secondary antibody conjugated to horse radish peroxidase (1:2,000, Bio-Rad Laboratories, 1721019) in PBST for 1 h at room temperature, washed three times for 15 min with PSBT, and then imaged as previously described. To quantify the bands, the Western blots were analyzed using Fiji (Schindelin et al., 2012). The intensity of each band was measured through Fiji's automatic measuring tool. The background surrounding each band was measured the same way and subtracted from the band intensity. The resulting band intensities were then made into a ratio of ZIP12 band intensity/GAPDH band intensity.

### Stereotaxic Surgeries

Male and female WT and ZnT3 KO mice at P21-P28 were anesthetized with inhaled isoflurane (induction, 3% in oxygen; maintenance, 1.5% in oxygen) and secured in a stereotaxic frame (Stoelting). Core body temperature was maintained at ~37° C. with a heating pad, and eyes were protected with ophthalmic ointment. Lidocaine (1%) was injected under the scalp, and an incision was made into the skin at the midline to expose the skull. Using a 27 gauge needle as a scalpel, a small craniotomy (~0.4 mm diameter) was made over the inferior colliculus at coordinates 1.3 mm posterior and 1.0 mm lateral to the lambda. Borosilicate glass pipettes (VWR International) were pulled to a shallow taper (length, >1 cm; tip diameter, ~30 μm) and advanced into the region of interest at an angle ~25° off the horizontal plane. Injection pipettes were backfilled with mineral oil (Sigma-Aldrich) and filled with pGP-AAV-syn-jGCaMP8m-WPRE (titer 5e12-5e13 genome copies/mL, Addgene) or cholera toxin subunit B conjugated to Alexa Fluor 555 (CTB-555, 1 mg/μl, Thermo Fisher Scientific). They were connected to 5 μl glass syringes (Hamilton) via capillary tubing and controlled with syringe pumps (World Precision Instruments). Pipettes were inserted 1.5 mm deep into the craniotomy, and 0.4 μl of CTB-555 was injected at 0.2 μl per minute for 2 min, or 0.9 μl of AAV was injected at 0.3 μl per minute for 3 min. After injections, the pipettes were left in place for 2 min prior to removal, and then the scalp of the mouse was closed with cyanoacrylate adhesive. Mice received an injection of nonsteroidal anti-inflammatory drug meloxicam during the injection procedure and a diet of meloxicam tablets (Bio-Serv) for 72 h after surgery. Mice were monitored for signs of postoperative stress and pain.

### Brain Slice Electrophysiology

Acute brain slice experiments using WT and ZnT3 KO (P25-P35) male and female mice were performed as previously described (Bender et al., 2023). Slices were examined during experiments to confirm accurate placement of the injection sites. Brain slices of ACx were cut in chilled carbogenated choline-based solution of the following composition (in mM): 110 choline chloride, 25 NaHCO₃, 25 D-glucose, 11.6 sodium ascorbate, 3.1 sodium pyruvate, 2.5 KCl, 0.5 CaCl2, 7 MgCl2. AMPA receptor miniature excitatory postsynaptic current (mEPSC) experiments were carried out using carbogenated ACSF with the following composition (in mM): 130 NaCl, 3 KCl, 2.4 CaCl2, 1.3MgCl2, 20NaHCO3, 3HEPES, 10D-glucose, 0.0005 tetrodotoxin (TTX), saturated with 95% O2/5% CO2 (vol/vol), pH 7.25-7.35, ~300 mOsm. All solutions were continuously bubbled with carbogen. Contaminating zinc was removed from the ACSF for all experiments by stirring the ACSF with Chelex 100 resin (Bio-Rad Laboratories) for 1 h. High-purity CaCl2 and MgCl2 salts (99.995% purity; Sigma-Aldrich) were added to the ACSF after the Chelex resin was filtered using Nalgene rapid flow filters lined with polyethersulfone (0.2 μM pore size). All plastic and glassware were washed with 5% high-purity nitric acid. Mice were first anesthetized with isoflurane and then immediately decapitated. Brains were rapidly removed, and coronal slices (300 μm) of the cortex were prepared in chilled choline chloride cutting solution using a vibratome (VT1200 S; Leica Biosystems). Slices were then transferred into a holding chamber of carbogenated ACSF and incubated for ~30 min at 35° C. and then incubated at room temperature for ~30 min before electrophysiological experiments were performed. For electrophysiological experiments, slices were transferred into the recording chamber and perfused with carbogenated ACSF at a rate of 1-2 ml/min. Recordings were performed at 30-32° C. using an in-line heating system (Warner Instruments). Corticocollicular neurons were identified by their labeling with the retrograde labeler cholera toxin subunit B conjugated to Alexa Fluor 555 (Thermo Fisher Scientific). Electrophysiological recordings were made using an amplifier (MultiClamp-700B, Axon Instruments), a digital to analog converter (USB-6229, National Instruments), and ephus (Suter et al., 2010). Voltage-clamp recordings were conducted using borosilicate pipettes (Warner Instruments) pulled to tip resistances of 3-5 MΩ (Sutter Instrument) filled with a cesium-based internal solution with the following composition (in mM): 128 cesium-methanosulfonate, 10 HEPES, 4 MgCl2, 4 Na, 2 ATP, 0.3 Tris-GTP, 10 Tris-phosphocreatine, 0.5 cesium-EGTA, 3 Na-ascorbate, 1 QX314, pH 7.23 (303 mOsm) at −70 mV holding. Series (Rₛₑᵣᵢₑₛ) and input resistance (Rᵢₙₚᵤₜ) were determined in a voltage-clamp mode (command potential set at −70 mV) by giving a −5 mV voltage step, which resulted in transient current responses. Rₛₑᵣᵢₑₛ was determined by dividing the −5 mV voltage step by the peak current value generated immediately after the step in the command potential. The difference between the baseline and steady-state hyperpolarized current (ΔI) was used to calculate Rᵢₙₚᵤₜ using the following formula: Rᵢₙₚᵤₜ=−5 mV/ΔI−Rₛₑᵣᵢₑₛ. Membrane time constant (τ; in msec) is the weighted time constant calculated via fitting a double exponential to the decay phase of the test step. Cell capacitance (in picofarads) was calculated by dividing the time constant by the input resistance. Current density was calculated as the holding current divided by the capacitance of the cell. For tonic NMDA receptor current experiments, a modified low-magnesium ACSF was used, with the following composition (in mM): 130 NaCl, 3 KCl, 2.4 CaCl2, 0.05 MgCl2, 20 NaHCO₃, 3 HEPES, 10 D-glucose, 0.0005 TTX, 0.02 SR95531 (gabazine), 0.025 DNQX, and 0.01 glycine, saturated with 95% O2/5% CO2 (vol/vol), pH 7.25-7.35, ~300 mOsm. AP5 (50 μM), ZX1 (300 μM), ZiMo12.8 (10 μM), and ZiMo12.9 (10 μM) were bath applied where indicated. ZiMo12.8 and ZiMo12.9 were dissolved in DMSO.

### Statistical Analysis

Analysis was performed with Fiji (Schindelin et al., 2012), MATLAB (MathWorks), and Prism (GraphPad). For statistical comparisons, a Student unpaired t test was used if the group data passed Lilliefors test for normality. If the group data were not normally distributed, a Kruskal-Wallis one-way ANOVA was used. A statistical comparison was determined to be significant if the p value was <0.05 for single comparisons or by use of Dunn's multiple-comparison test for multiple comparisons. Statistically significant differences are denoted in figures with an asterisk (*), and p values are listed in the figure legends. Statistical analyses were performed using Prism (GraphPad), MATLAB (MathWorks), and Excel (Microsoft). Bar plots show the mean with error bars representing ±standard error of the mean (SEM). Detailed information about the statistical tests used are available in Extended Data Table 1-1.

### c) Results

To investigate the potential roles of ZIP12 in synaptic function, first assessed is the expression pattern of ZIP12 protein in the brain. Single-cell sequencing studies have consistently found that ZIP12 mRNA expression is highly enriched in astrocytes compared with other types of brain cells (FIG. 1A; Zhang et al., 2014b; Zeisel et al., 2015; Clarke et al., 2018; Yao et al., 2021). Since ZIP12 is a zinc transporter protein that targets the plasma membrane (Chowanadisai et al., 2013), it was hypothesized that ZIP12 protein would be expressed by astrocytes in the brain and would be localized to synapses. To address these hypotheses, immunohistochemical staining for ZIP12 is performed in brain sections of mouse ACx (see Materials and Methods; FIG. 1B). Along with ZIP12, also stained for are the synaptic proteins bassoon (a presynaptic protein that assists in tethering vesicles at the active zone of synapses; Takao-Rikitsu et al., 2004; tom Dieck et al., 2005; Magupalli et al., 2008) and excitatory amino acid transporter 1 (EAAT1; an astrocytic protein that is responsible for glutamate reuptake into astrocytes after it is released from presynaptic terminals; Bunch et al., 2009; FIG. 1B). It was hypothesized that if EAAT1, bassoon, and ZIP12 are also closely associated, this would support the role of astrocytic ZIP12 expression at synapses. These relationships were quantified and it is and found that, consistent with the hypothesis, EAAT1 was closely associated with ZIP12 and bassoon (FIG. 1C), suggesting that EAAT1 and ZIP12 are proteins expressed in astrocytic processes near the active zone of synapses (Takao-Rikitsu et al., 2004; tom Dieck et al., 2005; Magupalli et al., 2008). Next, it was hypothesized that if astrocytic ZIP12 interacts with synaptically released zinc at synapses, ZIP12 and ZnT3 would also show a high degree of colocalization. To test this hypothesis, staining for ZnT3, ZIP12, and bassoon as above was performed. Found was a significant association of ZIP12 with ZnT3 with bassoon (FIG. 1D,E) in the cortex, suggesting that ZIP12 and ZnT3 might function to support synaptic zinc release and clearance. Together, these results suggest that astrocytes express the transporter proteins ZIP12 and EAAT1 which interact with zinc and glutamate in synaptic terminals after they are released from presynaptic vesicles during synaptic transmission.

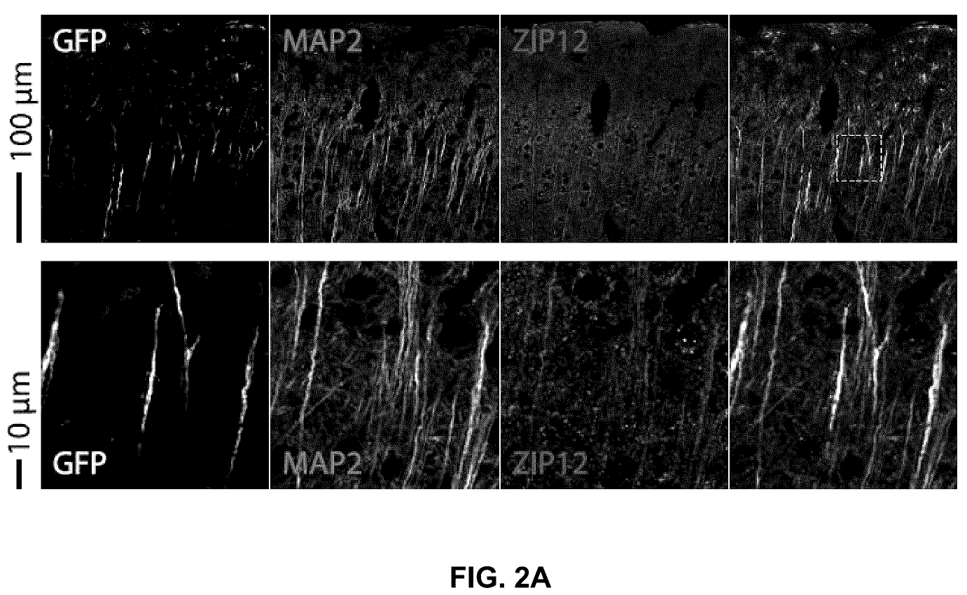

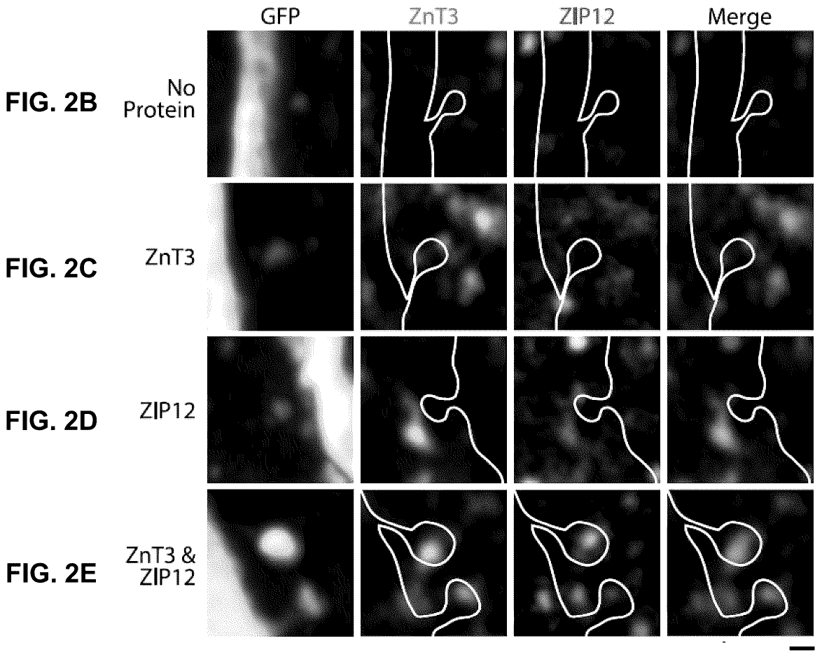

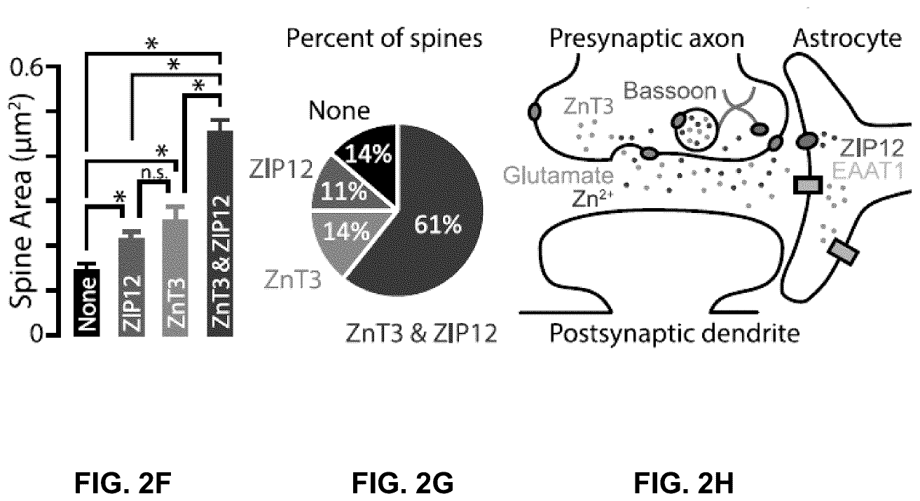

Given the association between ZnT3 and ZIP12 puncta in the brain, it was hypothesized that these proteins would localize to postsynaptic structures. To address this, stereotaxic injections of a retrograde AAV-GCaMP8m (see Materials and Methods) into the right inferior colliculus of WT mice were performed to induce the expression of GCaMP8 min Layer 5 corticocollicular neurons in the ACx (FIG. 2A). Mouse cortical sections were stained for MAP2 and ZIP12 to orient ZIP12 to the dendritic organization of GFP-expressing neurons in the cortex (FIG. 2A). We next quantified the relationship of ZIP12 and ZnT3 with postsynaptic dendritic spines in the cortex with antibodies for ZIP12 and ZnT3 (see Materials and Methods). Using GFP fluorescence, dendritic spines in layer 2/3 of the cortex were imaged and analyzed using a threshold measure of the spine head area (FIG. 2B-F; Hruska et al., 2022; Manning et al., 2024). Consistent with the association of ZnT3 with larger dendritic spines in the cortex (Manning et al., 2024), ZIP12 was also expressed at larger dendritic spines (FIG. 2F), with a majority of dendritic spines located at synapses that coexpress astrocytic ZIP12 and neuronal ZnT3 (FIG. 2G,H). Together, these results show that presynaptic ZnT3 and astrocytic ZIP12 are both associated with postsynaptic dendritic spines in the cortex, suggesting that these proteins are functionally coordinated with each other at synapses.

Having established that ZIP12 is expressed at synapses in the mouse cortex, we next sought methods to assess the functional consequences of ZIP12 on synaptic signaling. Specifically, we were interested in a method to reduce ZIP12 function because this could have therapeutic applications in disease models related to upregulation of ZIP12 expression, such as schizophrenia (Scarr et al., 2016; Dean et al., 2024). As a first step toward this goal, we established a ZIP12 activity assay based on heterologous expression systems that have been previously used to study the function of zinc transporters such as ZIP4 (Zhang et al., 2016, 2017; Hoch et al., 2020), ZIP13 (Bin et al., 2011), and ZnT5 Ohana et al., 2009). HEK293 cells were transfected with human Myc-tagged ZIP12 cDNA, and zinc uptake was determined using a fluorescent indicator in both transfected and native cells (Hoch et al., 2020). Transfection with ZIP12 cDNA resulted in increased ZIP12 expression by HEK293 cells (FIG. 3A). We quantified zinc uptake in HEK293 cells with FluoZin-3, AM—an intracellular, Zn2+-selective indicator that increases in fluorescence when bound to zinc (Hoch et al., 2020). 0.5 μM ZnCl2 was added to both native and ZIP12-transfected HEK293 cells (FIG. 3B). Consistent with expression of ZIP12 resulting in increased zinc transport into HEK293 cells, zinc application to cells transfected with ZIP12 cDNA resulted in increased fluorescent intensity compared with native cells (FIG. 3C). Having established a ZIP12 transport assay, we screened 610 compounds (1 μM) from a small-molecule library (Hit2Lead, ChemBridge; FIG. 3D,E) on ZIP12-transfected HEK293 cells. We quantified the effects of these putative ZIP12 modulator (ZiMo12) compounds on ZIP12 function by normalizing the changes in FluoZin-3 fluorescence compared with vehicle in ZIP12-transfected HEK293 cells and ranked the effect of the compounds from most antagonistic to most agonistic (FIG. 3E). Next, we sought to identify structural similarities between antagonist compounds by performing a virtual docking experiment combined with SAR analysis. Virtual docking experiments were performed by docking compounds into a putative binding site on the ZIP12 dimerization interface (see Materials and Methods; FIG. 3F,G,H; Extended Data Table 2-1). We focused on this dimerization interface because these residues are crucial for the homodimerization and zinc transport capacity of ZIP4, another member of the LIV-1 family of zinc transporter proteins and close homolog to ZIP12 (Kambe et al., 2015; Zhang et al., 2016, 2017). The dimerization interface was targeted by homology modeling in which the sequence of ZIP12 was mapped onto the structure of ZIP4 (FIG. 3F,G). The results showed that compounds with the highest inhibition and highest predicted binding affinity were consistently bound to the linker region connecting the ECD of ZIP12. These compounds all contained four cyclic rings which were generally nonpolar while also having relatively high flexibility owing to separation of cyclic structures. Based on these results, four compounds with high predicted binding affinity to ZIP12 and that experimentally acted as antagonists were used in further trials to assess ZIP12 specificity (FIG. 3I,J). To assess specificity of these compounds on ZIP12 versus other proteins expressed by HEK293 cells, we performed additional experiments comparing the effects of these compounds in native HEK293 cells versus ZIP12-transfected HEK293 cells. With these additional trials, the membranepermeable zinc chelator TPA was added at the end of the imaging session to return the fluorescence back to the baseline, ensuring that the fluorescence signals were zinc-dependent. Both transfected and native HEK293 cells underwent the zinc uptake assay in the presence of either vehicle or one of the top compounds (FIG. 3J). Three of these four compounds were able to reduce the zinc-mediated increase in FluoZin-3 fluorescence of ZIP12-transfected HEK293 cells to levels similar to untransfected HEK293 cells, suggesting that they are specifically acting to reduce the zinc transport capacity of ZIP12 expressed by transfected HEK293 cells (FIG. 3K). Together, these results suggest that the effects of these compounds on FluoZin-3 fluorescence are ZIP12 expression-dependent and are not due to other mechanisms—such as directly chelating zinc or directly affecting FluoZin-3 fluorescence—in this screening assay.

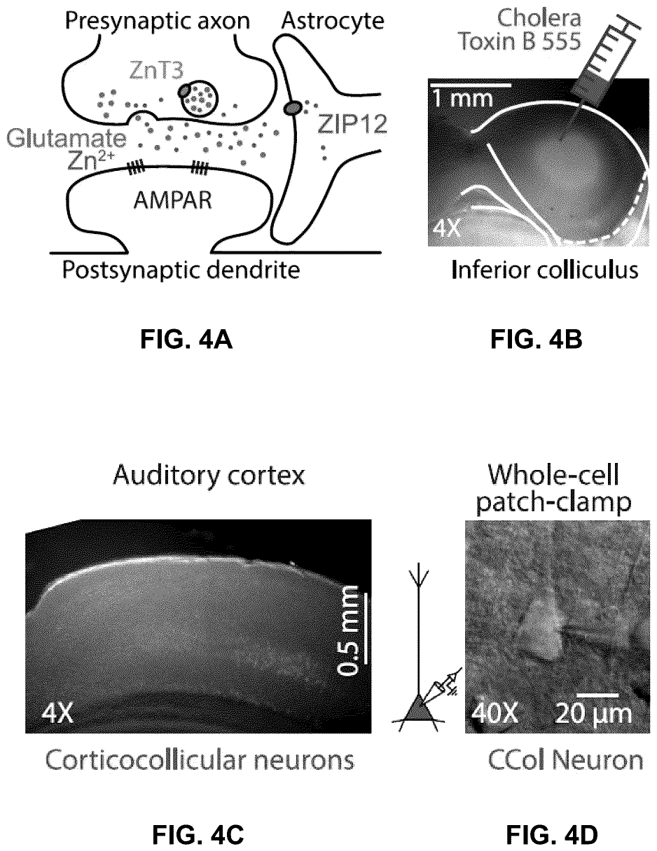

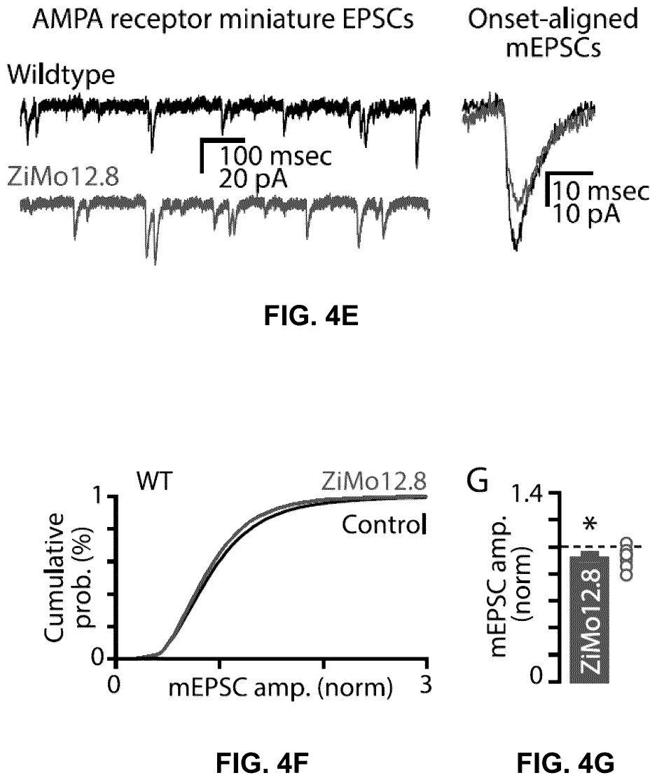

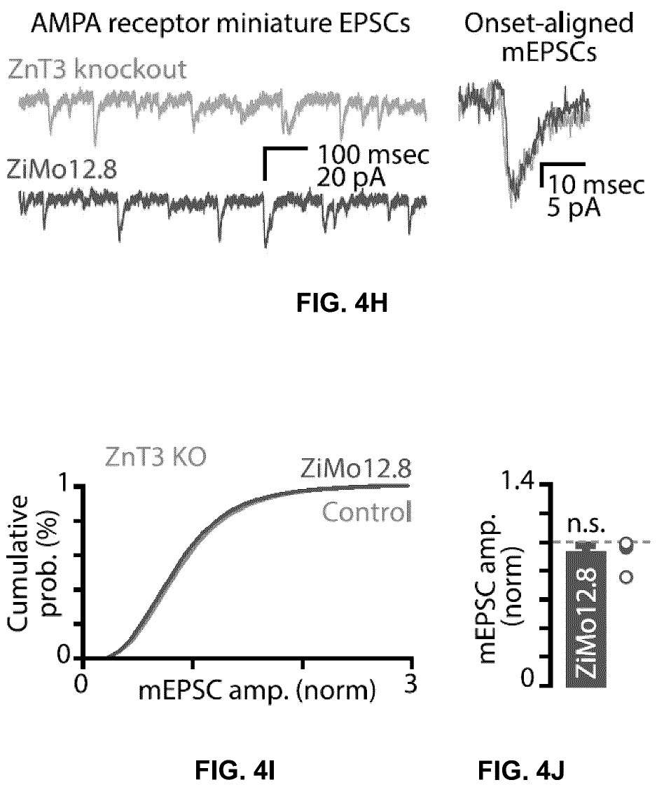

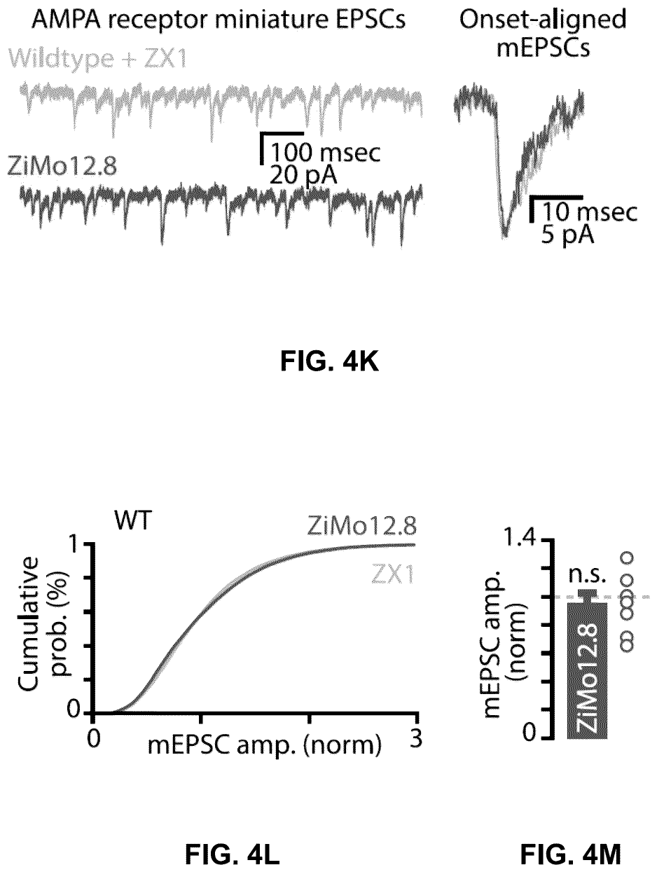

Having identified putative ZIP12 antagonists using SAR analysis combined with heterologous expression systems, we next set out to assess the effect of ZIP12 antagonist compounds on synaptic function. Because ZiMo12.8 had the largest effect on FluoZin-3 fluorescence in ZIP12-expressing HEK293 cells (28% signal reduction; FIG. 3K) and had the smallest effect on native HEK293 cells (1% signal reduction; FIG. 3K), we focused on this compound for experiments in acute brain slices of mouse ACx. It was hypothesized that if ZIP12 contributes to synaptic zinc clearance following its release into the synaptic cleft, ZiMo12.8 could increase zinc levels by reducing the zinc transport capacity of ZIP12 (FIG. 4A) and thereby increase zinc inhibition of glutamate receptors (McAllister and Dyck, 2017b). To test this hypothesis, 5 corticocollicular neurons were labeled by performing a stereotaxic injection of retrograde axonal tracer cholera toxin subunit B conjugated to Alexa Fluor 555 (CTB-555) into the right inferior colliculus of mice (FIG. 4B,C). Acute brain slices containing the ACx and visually targeted fluorescent neurons for electrophysiological recordings were then prepared. Whole-cell patch-clamp recordings were performed under voltage-clamp conditions in the presence of TTX to block sodium channels and isolate action potential-independent AMPA receptor-mediated miniature excitatory postsynaptic currents (mEPSCs; FIG. 4D). The addition of ZiMo12.8 resulted in a significant decrease in AMPA receptor mEPSC amplitudes, consistent with increased zinc levels in the cleft that became inhibitory to AMPA receptors (Blakemore and Trombley, 2004, 2019) by reducing the zinc transport activity of ZIP12 (FIG. 4E-G). The effects of ZiMo12.8 were dependent on the presence of synaptic zinc, because similar experiments using ZiMo12.8 in ZnT3 KO mice-which lack synaptic zinc-resulted in no changes to AMPA receptor mEPSC amplitudes (FIG. 4H-J). Furthermore, inWTmice, the high-affinity, extracellular, zinc-specific chelator ZX1 (Pan et al., 2011; Anderson et al., 2015; Kalappa et al., 2015; Morabito et al., 2022; Upmanyu et al., 2022; Bender et al., 2023) occluded the effects of ZiMo12.8 on mEPSC amplitudes. Together, these results suggest that the effects of ZiMo12.8 on AMPA receptor mEPSCs are due to increasing extracellular ZnT3-dependent zinc levels (FIG. 4K-M), that these effects can be occluded by genetic or pharmacological removal of zinc from synapses, and that ZiMo12.8 does not act directly on AMPA receptors in the absence of zinc.

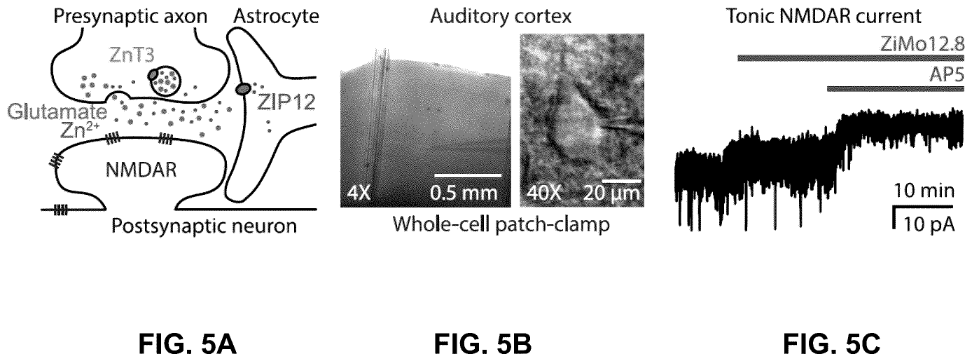

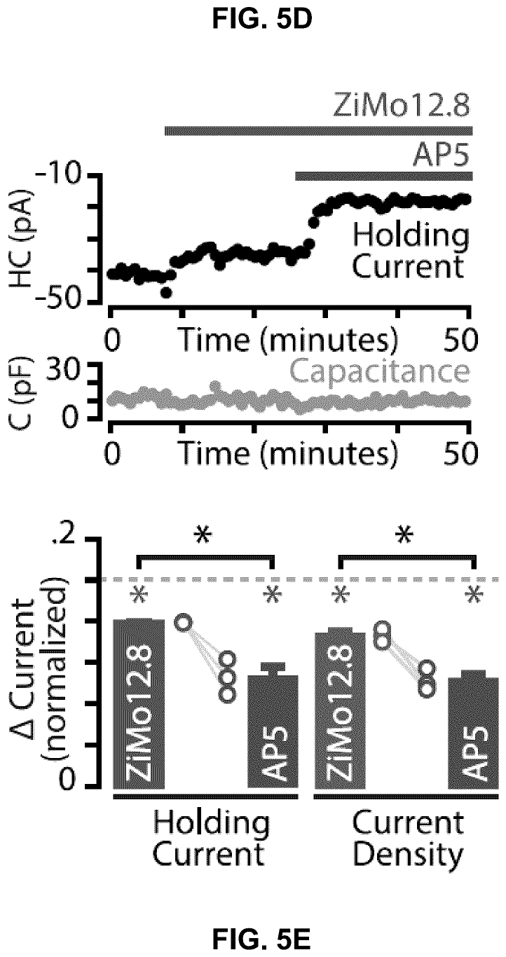

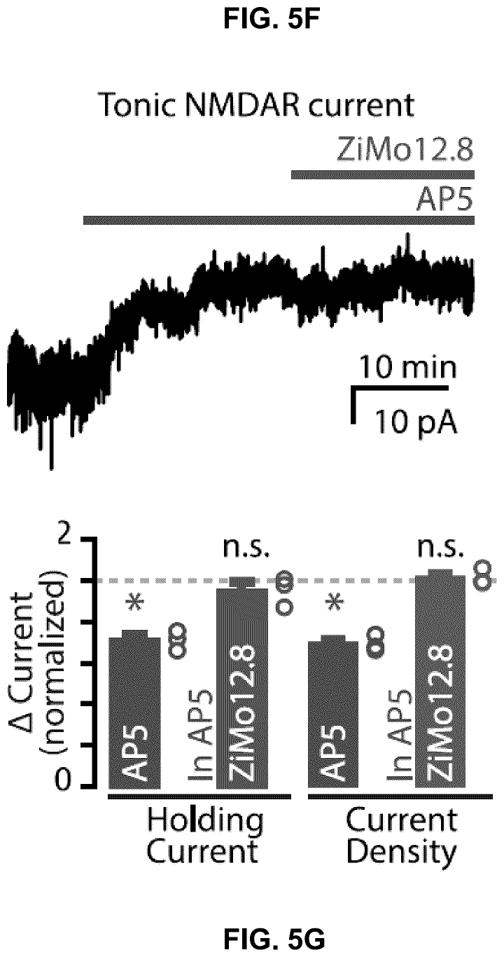

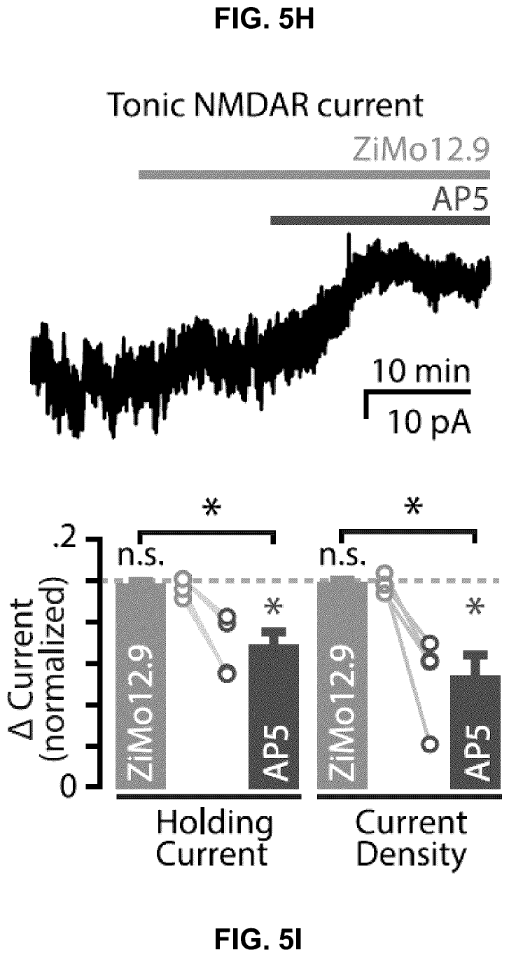

These electrophysiological results are consistent with ZiMo12.8 causing a ZnT3-dependent, ZX1-sensitive increase in extracellular zinc levels at synaptic connections in the brain. However, because changing zinc levels can have a range of effects on AMPA receptor function (from enhancement, to suppression, to no effect; Blakemore and Trombley, 2004; Pan et al., 2011; Kalappa et al., 2015; Blakemore and Trombley, 2019; Bender et al., 2023), it was hypothesized that the summation of these different effects of zinc on AMPA receptor function combine into the small but significant decrease in mEPSC amplitudes by ZiMo12.8. Although these results are consistent with an increase in synaptic zinc levels by ZiMol2.8, the range of possible effects of increasing zinc levels on AMPA receptor function is a potential caveat to this interpretation. To address this, a complimentary approach was utilized to assess the effects of ZiMo12.8 on zinc levels in the brain. Because NMDA receptors are unidirectionally inhibited by increased zinc levels (Paoletti et al., 1997; Rachline et al., 2005; Tovar and Westbrook, 2012; Hansen et al., 2014), it was hypothesized that if the inhibition of AMPA receptor function observed is due to ZiMo12.8 increasing zinc levels, this increase in zinc should also cause inhibition of NMDA receptor function. To test this hypothesis, whole-cell patch-clamp recordings were performed under voltage-clamp conditions in ACSF containing low levels of magnesium to remove the NMDA pore block by magnesium at negative membrane potentials (Mayer et al., 1984; Bender et al., 2023). The ACSF also contained TTX, SR95531 (gabazine), the AMPA receptor antagonist DNQX, and the NMDA receptor agonist glycine in order to isolate NMDA receptor-mediated tonic currents (Povysheva and Johnson, 2012). The addition of ZiMo12.8 decreased the tonic currents (FIG. 5A-E), consistent with ZiMo12.8 increasing zinc inhibition of NMDA receptors (Paoletti et al., 1997; Rachline et al., 2005; Tovar and Westbrook, 2012; Hansen et al., 2014). AP5 further reduced these currents showing that they are mediated by NMDA receptors (FIG. 5C-E). In experiments where AP5 was bath applied before ZiMo12.8, AP5 occluded the effect of ZiMo12.8 on tonic currents (FIG. 5F,G), confirming that the inhibition of tonic currents by ZiMo12.8 depends on the function of NMDA receptors. Importantly, these effects of ZiMo12.8 were also observed when current density was used as the measurement of tonic NMDA receptor activity (Lu et al., 2006; Gall and Dupont, 2019; Wu et al., 2021; Wyroslak et al., 2023) suggesting that the effects are due to zinc inhibition of NMDAR and not due to changes in the cell capacitance in response to ZiMo12.8. It was next hypothesized that if the effects of ZiMo12.8 result from ZIP12 antagonism, a different small molecule with low binding affinity for ZIP12 would not increase zinc-dependent inhibition of NMDA receptors. Therefore, similar experiments were performed with ZiMo12.9—which had no effect on HEK293 cell ZIP12-dependent zinc transport (FIG. 3D,E) and had low binding affinity for ZIP12 (FIG. 3H). Consistent with the hypothesis, ZiMo12.9 had no effect on tonic NMDA receptor currents in neurons (FIG. 5H,I). This result indicates that the inhibition of tonic NMDA receptor currents by ZiMo12.8 is not a general effect of small molecules, the vehicle used to deliver it into the ACSF, or due to other factors such as the duration of the whole cell recordings or the dialysis of neuronal intracellular milieu that occurs in these experiments. Taken together, these electrophysiological results suggest that ZiMo12.8 acts to increase levels of synaptically released zinc in the extracellular space via decreasing the zinc transport activity of ZIP12 in the brain.

### d) Discussion

As the importance of synaptic zinc in normal brain function is becoming more evident, there is a crucial need to understand the cellular and molecular mechanisms that control the dynamics of this powerful signaling system. This need is made more urgent because of the growing body of evidence that links altered synaptic zinc signaling to neurological conditions such as autism (Yoo et al., 2016), schizophrenia (Carrera et al., 2012; Perez-Becerril et al., 2016), and Alzheimer's disease (Lee et al., 2002; Beyer et al., 2009; Adlard et al., 2010) suggesting that synaptic zinc signaling could provide novel targets for therapeutic approaches in the treatment of these disorders. While the importance of ZnT3 for loading zinc ions into synaptic vesicles is well established (Palmiter et al., 1996; Kantheti et al., 1998; Cole et al., 1999; 2001; Vogt et al., 2000; Salazar et al., 2005, 2009; Upmanyu et al., 2022), much less is known about the molecular machinery that controls synaptic zinc levels during synaptic transmission. Here, ZIP12 was identified as a protein that contributes to extracellular zinc levels during synaptic transmission.

Synaptic zinc as a potent neuromodulatory signaling system (Pan et al., 2011; Vergnano et al., 2014; Anderson et al., 2015; Kalappa et al., 2015; Kouvaros et al., 2020; Cody and Tzounopoulos, 2022) that supports normal sensory processing (Anderson et al., 2017; Wu and Dyck, 2018; Kumar et al., 2019; Bender et al., 2023; McCollum et al., 2024) and ZnT3 KO mice (which lack synaptic zinc) display a range of cognitive, sensory, and behavioral deficits (Adlard et al., 2010; Martel et al., 2011; Sindreu et al., 2011; Yoo et al., 2016; Thackray et al., 2017; Wu and Dyck, 2018; Kumar et al., 2019; Zong et al., 2024). Synaptic zinc affects multiple aspects of excitatory and inhibitory neurotransmission. Exogenous zinc is an allosteric modulator that inhibits NMDA receptors (Legendre and Westbrook, 1990), with GluN2A-containing NMDA receptors inhibited by nanomolar levels of zinc (Paoletti et al., 1997; Tovar and Westbrook, 2012; Hansen et al., 2014). More recent work demonstrates that endogenous, synaptically released zinc inhibits NMDA receptors and affects synaptic plasticity (Vogt et al., 2000; Pan et al., 2011; Vergnano et al., 2014; Anderson et al., 2015). Synaptic zinc inhibits or potentiates AMPA receptor function (Kalappa et al., 2015; Bender et al., 2023) and triggers endocannabinoid synthesis that acts as a retrograde messenger to reduce presynaptic vesicular release probability (Perez-Rosello et al., 2013) and regulates chloride gradients and GABA signaling (Chorin et al., 2011) via the actions of the metabotropic zinc receptor GPR39 (Hershfinkel, 2018). At different concentrations, zinc also allosterically inhibits GABA receptors (Barberis et al., 2000), inhibits or potentiates glycine receptor function (Trombley et al., 2011; Perez-Rosello et al., 2015), and potentiates kainate glutamate receptors (Veran et al., 2012). The results uncover additional synaptic mechanisms that can contribute to the time course and concentration of zinc during synaptic transmission, which are crucial for determining the physiologically relevant roles of zinc at specific synapses in the brain.

The results add to the growing list of zinc transporter proteins that contribute to synaptic signaling in the brain. Recent work has clearly shown that ZnT1 associates with GluN2A-containing NMDA receptors and contributes to zinc inhibition of these glutamate receptors at synaptic connections in the central nervous system (Krall et al., 2020). In addition, ZIP4 expressed by neurons forms close associations with synaptic proteins in the dendrites of cultured neurons (De Benedictis et al., 2021). Thus, the findings that the astrocytic protein ZIP12 is also a synaptic protein increase the compliment of zinc transporter proteins that support synaptic physiology and synaptic zinc signaling. Together, these results extend the network of synaptic proteins in the brain that coordinate the effects of synaptic zinc on synaptic transmission and further expand the repertoire of astrocytic proteins that support synaptic function.

In this example, ZIP12 was identified as a synaptic protein and targeted its function at mouse cortical synapses. Pharmacological agents to selectively interfere with the removal of neurotransmitters from the synaptic cleft are powerful tools used for investigating synaptic function. From acetylcholinesterase inhibitors (Colović et al., 2013), glutamate reuptake inhibitors (Shimamoto et al., 1998), and GABA reuptake inhibitors (Braestrup et al., 1990), advancements in the understanding of synaptic physiology based on these tools have contributed to the understanding of synaptic function. This study identifies another synaptic protein to target with pharmacological agents to affect brain function. The zinc transporter system is an appealing target for neuromodulation because of its synapse specificity (Zeisel et al., 2015; Yao et al., 2021; Bender et al., 2023). Since not all synapses contain ZnT3 (Manning et al., 2024) or the same number or composition of AMPA and NMDA receptors (Nusser et al., 1998; Nusser, 2000; Masugi-Tokita et al., 2007), it is likely that zinc can have a diverse range of effects based on synapse-specific zinc release and clearance and on the specific expression of glutamate receptors. Unlike agents that target NMDA receptors such as ketamine and memantine (Johnson et al., 2015; Zhang et al., 2021; Chou et al., 2022; Wilcox et al., 2022; Ma et al., 2023) which are widely expressed throughout the nervous system, ZIP12 modulators have the potential to selectively affect glutamate receptors at synapses that express both ZnT3 and ZIP12, offering the ability to more precisely target certain synapses in the brain. Future studies establishing the specificity and potency of pharmacological compounds for ZIP12 over other zinc transporters (such as ZIP4; Kambe et al., 2015) will be required to more fully interpret the effects of ZIP12 antagonists on synaptic signaling and brain function. The present results show that the effects of ZiMo12.8 depend on the presence of ZIP12, ZnT3, extracellular zinc, and glutamate receptors, but do not rule out possible effects of ZiMo12.8 on other protein targets. The lack of effect of ZiMo12.8 on zinc levels in native HEK293 cells suggests that this compound has specific actions on ZIP12; however, future experiments to characterize the potency and specificity of ZiMo12.8 for ZIP12 will be required to understand and address potential off-target interactions of ZiMo12.8 with other proteins.

Astrocytes are integral components of synapses throughout the brain (Eroglu and Barres, 2010; Khakh and Sofroniew, 2015; Khakh and Deneen, 2019). They tile cortical space so that a single astrocyte supports the function of many types of synapses that occur within each astrocyte's spatial domain (Bushong et al., 2002, 2004; Baldwin et al., 2024). Astrocytes are sensitive to neurotransmitters such as GABA, acetylcholine, and adenosine triphosphate (Durkee and Araque, 2019) and can express neurotransmitter receptors such as muscarinic acetylcholine receptors, dopamine receptors, metabotropic GABA receptors, and purinergic receptors (Shan et al., 2021). Because they are also sensitive to glutamate via the actions of EAAT1 and EAAT2 (Chaudhry et al., 1995; Bergles et al., 1997; Diamond et al., 1998; Petr et al., 2015; Sipe et al., 2021) and metabotropic glutamate receptors (Panatier et al., 2011; Morel et al., 2014), they have simultaneous access to multiple modes of synaptic signaling that occur within their territory. Combined with their ability to generate intracellular calcium transients (Goenaga et al., 2023; Gau et al., 2024) that can propagate to neighboring astrocytes (Kuga et al., 2011; Fujii et al., 2017), there is an emerging appreciation for a signaling role of astrocytes in information processing (Papouin et al., 2017) and synaptic development (Sipe et al., 2021) and as additional pathways for information propagation in the brain (Araque et al., 2014). Together, the findings highlight another contribution of nonneuronal brain cells to synaptic function, further expanding the repertoire of how these cells can contribute to normal and pathological conditions relating to zinc signaling in the brain.

### REFERENCES RELATED TO EXAMPLE 1

Adlard P A, Parncutt J M, Finkelstein D I, Bush A I (2010) Cognitive loss in zinc transporter-3 knock-out mice: a phenocopy for the synaptic and memory deficits of Alzheimer's disease? J Neurosci 30:1631-1636.
Adlard P A, Parncutt J, Lal V, James S, Hare D, Doble P, Finkelstein D I, Bush A I (2015) Metal chaperones prevent zinc-mediated cognitive decline. Neurobiol Dis 81:196-202.
Adlard P A, Sedjahtera A, Gunawan L, Bray L, Hare D, Lear J, Doble P, Bush A I, Finkelstein D I, Cherny R A (2014) A novel approach to rapidly prevent age-related cognitive decline. Aging Cell 13:351-359.
Anderson C T, Kumar M, Xiong S, Tzounopoulos T (2017) Cell-specific gain modulation by synaptically released zinc in cortical circuits of audition. eLife 6:e29893.
Anderson C T, Radford R J, Zastrow M L, Zhang D Y, Apfel U P, Lippard S J, Tzounopoulos T (2015) Modulation of extrasynaptic NMDA receptors by synaptic and tonic zinc. Proc Natl Acad Sci USA 112:E2705-14.
Araque A, Carmignoto G, Haydon P G, Oliet S H, Robitaille R, Volterra A (2014) Gliotransmitters travel in time and space. Neuron 81:728-739.
Ashkenazy H, Abadi S, Martz E, Chay O, Mayrose I, Pupko T, Ben-Tal N (2016) Consurf 2016: an improved methodology to estimate and visualize evolutionary conservation in macromolecules. Nucleic acids research 44: W344-W350.
Baldwin K T, Murai K K, Khakh B S (2024) Astrocyte morphology. Trends Cell Biol 34:547-565.
Barberis A, Cherubini E, Mozrzymas J W (2000) Zinc inhibits miniature GABAergic currents by allosteric modulation of GABAA receptor gating. J Neurosci 20:8618-8627.
Bender P T R, McCollum M, Boyd-Pratt H, Mendelson B Z, Anderson C T (2023) Synaptic zinc potentiates AMPA receptor function in mouse auditory cortex. Cell Rep 42:112932.
Bergles D E, Dzubay J A, Jahr C E (1997) Glutamate transporter currents in Bergmann glial cells follow the time course of extrasynaptic glutamate. Proc Natl Acad Sci USA 94:14821-14825.
Beyer N, Coulson D T, Heggarty S, Ravid R, Irvine G B, Hellemans J, Johnston J A (2009) Znt3 mRNA levels are reduced in Alzheimer's disease postmortem brain. Mol Neurodegener 4:53.
Bin B H, Fukada T, Hosaka T, Yamasaki S, Ohashi W, Hojyo S, Miyai T, Nishida K, Yokoyama S, Hirano T (2011) Biochemical characterization of human ZIP13 protein: a homo-dimerized zinc transporter involved in the spondylocheiro dysplastic Ehlers-Danlos syndrome. J Biol Chem 286:40255-40265.
Blakemore L J, Trombley P Q (2004) Diverse modulation of olfactory bulb AMPA receptors by zinc. Neuroreport 15:919-923.
Blakemore L J, Trombley P Q (2019) Mechanisms of zinc modulation of olfactory bulb AMPA receptors. Neuroscience 410:160-175.
Boisvert M M, Erikson G A, Shokhirev M N, Allen N J (2018) The aging astrocyte transcriptome from multiple regions of the mouse brain. Cell Rep 22: 269-285.
Braestrup C, Nielsen E B, Sonnewald U, Knutsen L J, Andersen K E, Jansen J A, Frederiksen K, Andersen P H, Mortensen A, Suzdak P D (1990) (R)—N-[4,4-bis(3-methyl-2-thienyl)but-3-en-1-yl]nipecotic acid binds with high affinity to the brain gamma-aminobutyric acid uptake carrier. J Neurochem 54:639-647.
Bunch L, Erichsen M N, Jensen A A (2009) Excitatory amino acid transporters as potential drug targets. Expert Opin Ther Targets 13:719-731.
Bushong E A, Martone M E, Ellisman M H (2004) Maturation of astrocyte morphology and the establishment of astrocyte domains during postnatal hippocampal development. Int J Dev Neurosci 22:73-86.
Bushong E A, Martone M E, Jones Y Z, Ellisman M H (2002) Protoplasmic astrocytes in CA1 stratum radiatum occupy separate anatomical domains. J Neurosci 22:183-192.
Carrera N, et al. (2012) Association study of nonsynonymous single nucleotide polymorphisms in schizophrenia. Biol Psychiatry 71:169-177.
Chaudhry F A, Lehre K P, van Lookeren Campagne M, Ottersen O P, Danbolt N C, Storm-Mathisen J (1995) Glutamate transporters in glial plasma membranes: highly differentiated localizations revealed by quantitative ultrastructural immunocytochemistry. Neuron 15:711-720.
Chen X, Sun Y-C, Zhan H, Kebschull J M, Fischer S, Matho K, Huang Z J, Gillis J, Zador A M (2019) High-throughput mapping of long-range neuronal projection using in situ sequencing. Cell 179:772-786.e19.
Chorin E, Vinograd O, Fleidervish I, Gilad D, Herrmann S, Sekler I, Aizenman E, Hershfinkel M (2011) Upregulation of KCC2 activity by zinc-mediated neurotransmission via the mZnR/GPR39 receptor. J Neurosci 31:12916-12926.
Chou T H, Epstein M, Michalski K, Fine E, Biggin P C, Furukawa H (2022) Structural insights into binding of therapeutic channel blockers in NMDA receptors. Nat Struct Mol Biol 29:507-518.
Chowanadisai W, Graham D M, Keen C L, Rucker R B, Messerli M A (2013) Neurulation and neurite extension require the zinc transporter ZIP12 (slc39a12). Proc Natl Acad Sci USA 110:9903-9908.
Clarke L E, Liddelow S A, Chakraborty C, MQnch A E, Heiman M, Barres B A (2018) Normal aging induces A1-like astrocyte reactivity. Proc Natl Acad Sci USA 115:E1896-E1905.
Cody P A, Tzounopoulos T (2022) Neuromodulatory mechanisms underlying contrast gain control in mouse auditory cortex. J Neurosci 42:5564-5579.
Cole T B, Martyanova A, Palmiter R D (2001) Removing zinc from synaptic vesicles does not impair spatial learning, memory, or sensorimotor functions in the mouse. Brain Res 891:253-265.
Cole T B, Wenzel H J, Kafer K E, Schwartzkroin P A, Palmiter R D (1999) Elimination of zinc from synaptic vesicles in the intact mouse brain by disruption of the ZnT3 gene. Proc Natl Acad Sci USA 96:1716-1721.
Colović M B, Krstić D Z, Lazarević-Pašti T D, Bondžić A M, Vasić V M (2013) Acetylcholinesterase inhibitors: pharmacology and toxicology. Curr Neuropharmacol 11:315-335.
Dean B, Hopper S, Scarr E (2024) Changes in levels of the zinc transporter SLC39A12 in Brodmann's area 44: effects of sex, suicide, CNS pH and schizophrenia. J Psychiatr Res 177:177-184.
De Benedictis C A, Haffke C, Hagmeyer S, Sauer A K, Grabrucker A M (2021) Expression analysis of zinc transporters in nervous tissue cells reveals neuronal and synaptic localization of ZIP4. Int J Mol Sci 22:4511.
Diamond J S, Bergles D E, Jahr C E (1998) Glutamate release monitored with astrocyte transporter currents during LTP. Neuron 21:425-433.
Durkee C A, Araque A (2019) Diversity and specificity of astrocyte-neuron communication. Neuroscience 396:73-78.
Eberhardt J, Santos-Martins D, Tillack A F, Forli S (2021) AutoDock Vina 1.2.0: new docking methods, expanded force field, and python bindings. J Chem Inf Model 61:3891-3898.
Eroglu C, Barres B A (2010) Regulation of synaptic connectivity by glia. Nature 468:223-231.
Frederickson C J, Koh J-Y Y, Bush A I (2005) The neurobiology of zinc in health and disease. Nat Rev Neurosci 6:449-462.
Fujii Y, Maekawa S, Morita M (2017) Astrocyte calcium waves propagate proximally by gap junction and distally by extracellular diffusion of ATP released from volume-regulated anion channels. Sci Rep 7:13115.
Gall D, Dupont G (2019) Tonic activation of extrasynaptic NMDA receptors decreases intrinsic excitability and promotes bistability in a model of neuronal activity. Int J Mol Sci 21:206.
Gau Y A, Hsu E T, Cha R J, Pak R W, Looger L L, Kang J U, Bergles D E (2024) Multicore fiber optic imaging reveals that astrocyte calcium activity in the mouse cerebral cortex is modulated by internal motivational state. Nat Commun 15:3039.
Goenaga J, Araque A, Kofuji P, Herrera Moro Chao D (2023) Calcium signaling in astrocytes and gliotransmitter release. Front Synaptic Neurosci 15: 1138577.
Hansen K B, Ogden K K, Yuan H, Traynelis S F (2014) Distinct functional and pharmacological properties of Triheteromeric GluN1/GluN2A/GluN2B NMDA receptors. Neuron 81:1084-1096.
Hershfinkel M (2018) The zinc sensing receptor, ZnR/GPR39, in health and disease. Int J Mol Sci 19:439.
Hoch E, Levy M, Hershfinkel M, Sekler I (2020) Elucidating the H+ coupled Zn2+ transport mechanism of ZIP4; implications in acrodermatitis enteropathica. Int J Mol Sci 21:734.
Hruska M, Cain R E, Dalva M B (2022) Nanoscale rules governing the organization of glutamate receptors in spine synapses are subunit specific. Nat Commun 13:920.
Huang Z, Zhang X A, Bosch M, Smith S J, Lippard S J (2013) Tris(2-pyridylmethyl)amine (TPA) as a membrane-permeable chelator for interception of biological mobile zinc. Metallomics 5:648-655.
Icha J, Weber M, Waters J C, Norden C (2017) Phototoxicity in live fluorescence microscopy, and how to avoid it. Bioessays 39.
Insel T R (2010) Rethinking schizophrenia. Nature 468:187.
Johnson J W, GlasgowNG, Povysheva N V (2015) Recent insights into themode of action of memantine and ketamine. Curr Opin Pharmacol 20:54-63.
Kalappa B I, Anderson C T, Goldberg J M, Lippard S J, Tzounopoulos T (2015) AMPA receptor inhibition by synaptically released zinc. Proc Natl Acad Sci USA 112:15749-15754.
Kambe T, Tsuji T, Hashimoto A, Itsumura N (2015) The physiological, biochemical, and molecular roles of zinc transporters in zinc homeostasis and metabolism. Physiol Rev 95:749-784.
Kantheti P, Qiao X, Diaz M E, Peden A A, Meyer G E, Carskadon S L, Kapfhamer D, Sufalko D, Robinson M S, Noebels J L (1998) Mutation in AP-3 5 in the mocha mouse links endosomal transport to storage deficiency in platelets, melanosomes, and synaptic vesicles. Neuron 21: 111-122.
Khakh B S, Deneen B (2019) The emerging nature of astrocyte diversity. Annu Rev Neurosci 42:187-207.
Khakh B S, Sofroniew M V (2015) Diversity of astrocyte functions and phenotypes in neural circuits. Nat Neurosci 18:942-952.
Kouvaros S, Kumar M, Tzounopoulos T (2020) Synaptic zinc enhances inhibition mediated by somatostatin, but not parvalbumin, cells in mouse auditory cortex. Cereb Cortex 30:3895-3909.
Krall R F, Moutal A, Phillips M B, Asraf H, Johnson J W, Khanna R, Hershfinkel M, Aizenman E, Tzounopoulos T (2020) Synaptic zinc inhibition of NMDA receptors depends on the association of GluN2A with the zinc transporter ZnT1. Sci Adv 6:eabb1515.
Kuga N, Sasaki T, Takahara Y, Matsuki N, Ikegaya Y (2011) Large-scale calcium waves traveling through astrocytic networks in vivo. J Neurosci 31: 2607-2614.
Kumar M, Xiong S, Tzounopoulos T, Anderson C T (2019) Fine control of sound frequency tuning and frequency discrimination acuity by synaptic zinc signaling in mouse auditory cortex. J Neurosci 39:854-865.
Lee J Y, Cole T B, Palmiter R D, Suh S W, Koh J Y (2002) Contribution by synaptic zinc to the gender-disparate plaque formation in human Swedish mutant APP transgenic mice. Proc Natl Acad Sci USA 99:7705-7710.
Legendre P, Westbrook G L (1990) The inhibition of single N-methyl-Daspartate-activated channels by zinc ions on cultured rat neurones. J Physiol 429:429-449.
Lu C, Fu Z, Karavanov I, Yasuda R P, Wolfe B B, Buonanno A, Vicini S (2006) NMDA receptor subtypes at autaptic synapses of cerebellar granule neurons. J Neurophysiol 96:2282-2294.
Ma S, et al. (2023) Sustained antidepressant effect of ketamine through NMDAR trapping in the LHb. Nature 622:802-809.
Magupalli V G, Schwarz K, Alpadi K, Natarajan S, Seigel G M, Schmitz F (2008) Multiple RIBEYE-RIBEYE interactions create a dynamic scaffold for the formation of synaptic ribbons. J Neurosci 28:7954-7967.
Manning A, Bender P T R, Boyd-PrattH, Mendelson B Z, Hruska M, Anderson C T (2024) Trans-synaptic association of vesicular zinc transporter 3 and shank3 supports synapse-specific dendritic spine structure and function in the mouse auditory cortex. J Neurosci 44:e0619242024.
Martel G, Hevi C, Kane-Goldsmith N, Shumyatsky G P (2011) Zinc transporter ZnT3 is involved in memory dependent on the hippocampus and perirhinal cortex. Behav Brain Res 223:233-238.
Masugi-Tokita M, Tarusawa E, Watanabe M, Molner E, Fujimoto K, Shigemoto R (2007) Number and density of AMPA receptors in individual synapses in the rat cerebellum as revealed by SDS-digested freezefracture replica labeling. J Neurosci 27:2135-2144.
Mayer M L, Westbrook G L, Guthrie P B (1984) Voltage-dependent block by Mg2+ of NMDA responses in spinal cord neurones. Nature 309:261-263.
McAllister B B, Dyck R H (2017a) Sound processing: a new role for zinc in the brain. eLife 6:e31816.
McAllister B B, Dyck R H (2017b) Zinc transporter 3 (ZnT3) and vesicular zinc in central nervous system function. Neurosci Biobehav Rev 80:329-350.
McCollum M, Manning A, Bender P T R, Mendelson B Z, Anderson C T (2024) Cell-type-specific enhancement of deviance detection by synaptic zinc in the mouse auditory cortex. Proc Natl Acad Sci USA 121:e2405615121.
Morabito A, Zerlaut Y, Serraz B, Sala R, Paoletti P, Rebola N (2022) Activity-dependent modulation of NMDA receptors by endogenous zinc shapes dendritic function in cortical neurons. Cell Rep 38:110415.
Morel L, Higashimori H, Tolman M, Yang Y (2014) VGIuT1+ neuronal glutamatergic signaling regulates postnatal developmental maturation of cortical protoplasmic astroglia. J Neurosci 34:10950-10962.
Nishikawa M, Mori H, Hara M (2017) Analysis of ZIP (Zrt-, Irt-related protein) transporter gene expression in murine neural stem/progenitor cells. Environ Toxicol Pharmacol 53:81-88.
Nusser Z (2000) AMPA and NMDA receptors: similarities and differences in their synaptic distribution. Curr Opin Neurobiol 10:337-341.
Nusser Z, Lujan R, Laube G, Roberts J D, Molnar E, Somogyi P (1998) Cell type and pathway dependence of synaptic AMPA receptor number and variability in the hippocampus. Neuron 21:545-559.
Ohana E, Hoch E, Keasar C, Kambe T, Yifrach O, Hershfinkel M, Sekler I (2009) Identification of the Zn2+ binding site and mode of operation of a mammalian Zn2+ transporter. J Biol Chem 284:17677-17686.
Palmiter R D, Cole T B, Quaife C J, Findley S D (1996) ZnT-3, a putative transporter of zinc into synaptic vesicles. Proc Natl Acad Sci USA 93:14934-14939.
Pan E, Zhang X A, Huang Z, Krezel A, Zhao M, Tinberg C E, Lippard S J, McNamara J O (2011) Vesicular zinc promotes presynaptic and inhibits postsynaptic long-term potentiation of mossy fiber-CA3 synapse. Neuron 71:1116-1126.
Panatier A, Vallée J, Haber M, Murai K K, Lacaille J C, Robitaille R (2011) Astrocytes are endogenous regulators of basal transmission at central synapses. Cell 146:785-798.
Paoletti P, Ascher P, Neyton J (1997) High-affinity zinc inhibition of NMDA NR1-NR2A receptors. J Neurosci 17:5711-5725.
Papouin T, Dunphy J, TolmanM, Foley J C, Haydon P G (2017) Astrocytic control of synaptic function. Philos Trans R Soc Lond B Biol Sci 372:20160154.
Perea G, Navarrete M, Araque A (2009) Tripartite synapses: astrocytes process and control synaptic information. Trends Neurosci 32:421-431.
Perez-Becerril C, Morris A G, Mortimer A, McKenna P J, de Belleroche J (2016) Common variants in the chromosome 2p23 region containing the SLC30A3 (ZnT3) gene are associated with schizophrenia in female but not male individuals in a large collection of European samples. Psychiatry Res 246:335-340.
Perez-Rosello T, Anderson C T, Ling C, Lippard S J, Tzounopoulos T (2015) Tonic zinc inhibits spontaneous firing in dorsal cochlear nucleus principal neurons by enhancing glycinergic neurotransmission. Neurobiology of disease 81:14-19.
Perez-Rosello T, Anderson C T, Schopfer F J, Zhao Y, Gilad D, Salvatore S R, Freeman B A, Hershfinkel M, Aizenman E, Tzounopoulos T (2013) Synaptic Zn2+ inhibits neurotransmitter release by promoting endocannabinoid synthesis. J Neurosci 33:9259-9272.
Petr G T, et al. (2015) Conditional deletion of the glutamate transporter GLT-1 reveals that astrocytic GLT-1 protects against fatal epilepsy while neuronal GLT-1 contributes significantly to glutamate uptake into synaptosomes. J Neurosci 35:5187-5201.
Povysheva N V, Johnson J W (2012) Tonic NMDA receptor-mediated current in prefrontal cortical pyramidal cells and fast-spiking interneurons. J Neurophysiol 107:2232-2243.
Rachline J, Perin-Dureau F, Le Goff A, Neyton J, Paoletti P (2005) The micromolar zinc-binding domain on the NMDA receptor subunit NR2B. J Neurosci 25:308-317.
Rose C R, Felix L, Zeug A, Dietrich D, Reiner A, Henneberger C (2018) Astroglial glutamate signaling and uptake in the hippocampus. Front Mol Neurosci 10:451.
Salazar G, Craige B, Love R, Kalman D, Faundez V (2005) Vglut1 and ZnT3 co-targeting mechanisms regulate vesicular zinc stores in PC12 cells. J Cell Sci 118:1911-1921.
Salazar G, Falcon-Perez J M, Harrison R, Faundez V (2009) SLC30A3 (ZnT3) oligomerization by dityrosine bonds regulates its subcellular localization and metal transport capacity. PLoS One 4:e5896.
Sander T, Freyss J, von Korff M, Rufener C (2015) DataWarrior: an opensource program for chemistry aware data visualization and analysis. J Chem Inf Model 55:460-473.
Scarr E, et al. (2016) Increased cortical expression of the zinc transporter SLC39A12 suggests a breakdown in zinc cellular homeostasis as part of the pathophysiology of schizophrenia. NPJ Schizophr 2:16002.
Schindelin J, et al. (2012) Fiji: an open-source platform for biological-image analysis. Nat Methods 9:676-682.
Seve M, Chimienti F, Devergnas S, Favier A (2004) In silico identification and expression of SLC30 family genes: an expressed sequence tag data mining strategy for the characterization of zinc transporters' tissue expression. BMC genomics 5:32.
Shan L, Zhang T, Fan K, Cai W, Liu H (2021) Astrocyte-neuron signaling in synaptogenesis. Front Cell Dev Biol 9:680301.
Shimamoto K, Lebrun B, Yasuda-Kamatani Y, Sakaitani M, Shigeri Y, Yumoto N, Nakajima T (1998) D L-threo-beta-benzyloxyaspartate, a potent blocker of excitatory amino acid transporters. Mol Pharmacol 53:195-201.
Sindreu C, Palmiter R D, Storm D R (2011) Zinc transporter ZnT-3 regulates presynaptic Erk1/2 signaling and hippocampus-dependent memory. Proc Natl Acad Sci USA 108:3366-3370.
Sipe G O, Petravicz J, Rikhye R V, Garcia R, Mellios N, SurM(2021) Astrocyte glutamate uptake coordinates experience-dependent, eye-specific refinement in developing visual cortex. Glia 69:1723-1735.
Styrpejko D J, Cuajungco M P (2021) Transmembrane 163 (TMEM163) protein: a new member of the zinc efflux transporter family. Biomedicines 9: 220.
Surre J, Saint-Ruf C, Collin V, Orenga S, Ramjeet M, Matic I (2018) Strong increase in the autofluorescence of cells signals struggle for survival. Sci Rep 8:12088.
Suter B A, O'Connor T, lyer V, Petreanu L T, Hooks B M, Kiritani T, Svoboda K, Shepherd G M G (2010) Ephus: multipurpose data acquisition software for neuroscience experiments. Front Neural Circuits 4:100.
Takao-Rikitsu E, Mochida S, Inoue E, Deguchi-Tawarada M, Inoue M, Ohtsuka T, Takai Y (2004) Physical and functional interaction of the active zone proteins, CAST, RIM1, and Bassoon, in neurotransmitter release. J Cell Biol 164:301-311.
Thackray S E, McAllister B B, Dyck R H (2017) Behavioral characterization of female zinc transporter 3 (ZnT3) knockout mice. Behav Brain Res 321:36-49.
tom Dieck S, Altrock W D, Kessels M M, Qualmann B, Regus H, Brauner D, Fejtove A, Bracko O, Gundelfinger E D, Brandstatter J H (2005) Molecular dissection of the photoreceptor ribbon synapse: physical interaction of bassoon and RIBEYE is essential for the assembly of the ribbon complex. J Cell Biol 168:825-836.
Tovar K R, Westbrook G L (2012) Amino-terminal ligands prolong NMDA receptor-mediated EPSCs. J Neurosci 32:8065-8073.
Trombley P Q, Blakemore L J, Hill B J (2011) Zinc modulation of glycine receptors. Neuroscience 186:32-38.
Trott O, Olson A J (2010) AutoDock Vina: improving the speed and accuracy of docking with a new scoring function, efficient optimization, and multithreading. J Comput Chem 31:455-461.
Upmanyu N, et al. (2022) Colocalization of different neurotransmitter transporters on synaptic vesicles is sparse except for VGLUT1 and ZnT3. Neuron 110:1483-1497.e7.
Veran J, Kumar J, Pinheiro P S, Athane A, Mayer M L, Perrais D, Mulle C (2012) Zinc potentiates GluK3 glutamate receptor function by stabilizing the ligand binding domain dimer interface. Neuron 76:565-578.
Vergnano A M, Rebola N, Savtchenko L P, Pinheiro P S, Casado M, Kieffer B L, Rusakov D A, Mulle C, Paoletti P (2014) Zinc dynamics and action at excitatory synapses. Neuron 82:1101-1114.
Vogt K, Mellor J, Tong G, Nicoll R (2000) The actions of synaptically released zinc at hippocampal mossy fiber synapses. Neuron 26:187-196.
Wilcox M R, et al. (2022) Inhibition of NMDA receptors through a membrane-to-channel path. Nat Commun 13:4114.
Wu K, Castellano D, Tian Q, Lu W (2021) Distinct regulation of tonic GABAergic inhibition by NMDA receptor subtypes. Cell Rep 37:109960.
Wu H P, Dyck R H (2018) Signaling by synaptic zinc is required for whiskermediated, fine texture discrimination. Neuroscience 369:242-247.
Wyroslak M, Dobrzanski G, Mozrzymas J W (2023) Bidirectional plasticity of GABAergic tonic inhibition in hippocampal somatostatin- and parvalbumin-containing interneurons. Front Cell Neurosci 17: 1193383.
Yao Z, et al. (2021) A taxonomy of transcriptomic cell types across the isocortex and hippocampal formation. Cell 184:3222-3241.e26.
Yariv B, Yariv E, Kessel A, Masrati G, Chorin A B, Martz E, Mayrose I, Pupko T, Ben-Tal N (2023) Using evolutionary data to make sense of macromolecules with a “face-lifted” ConSurf. Protein Science 32:e4582.
Yoo M H, Kim T-Y Y, Yoon Y H, Koh J-Y Y (2016) Autism phenotypes in ZnT3 null mice: Involvement of zinc dyshomeostasis, MMP-9 activation and BDNF upregulation. Sci Rep 6:28548.
Zeisel A, Muñoz-Manchado A B, Codeluppi S, Lönnerberg P, La Manno G, Juréus A, Marques S, Munguba H, He L, Betsholtz C (2015) Cell types in the mouse cortex and hippocampus revealed by single-cell RNA-seq. Science 347:1138-1142.
Zhang Y, Chen K, Sloan S A, Bennett M L, Scholze A R, O'Keeffe S, Phatnani H P, Guarnieri P, Caneda C, Ruderisch N (2014a) An RNA-sequencing transcriptome and splicing database of glia, neurons, and vascular cells of the cerebral cortex. J Neurosci 34:11929-11947.
Zhang Y, Chen K, Sloan S A, Bennett M L, Scholze A R, O'Keeffe S, Phatnani H P, Guarnieri P, Caneda C, Ruderisch N (2014b) An RNA-sequencing transcriptome and splicing database of glia, neurons, and vascular cells of the cerebral cortex. J Neurosci 34:11929-11947.
Zhang T, Liu J, Fellner M, Zhang C, Sui D, Hu J (2017) Crystal structures of a ZIP zinc transporter reveal a binuclear metal center in the transport pathway. Science Advances 3:e1700344.
Zhang T, Sui D, Hu J (2016) Structural insights of ZIP4 extracellular domain critical for optimal zinc transport. Nat Commun 7:11979.
Zhang Y, Ye F, Zhang T, Lv S, Zhou L, Du D, Lin H, Guo F, Luo C, Zhu S (2021) Structural basis of ketamine action on human NMDA receptors. Nature 596:301-305.
Zong R, Zhang X, Dong X, Liu G, Zhang J, Gao Y, Zhang Z, Ma Y, Gao H, Gamper N (2024) Genetic deletion of zinc transporter ZnT3 induces progressive cognitive deficits in mice by impairing dendritic spine plasticity and glucose metabolism. Front Mol Neurosci 17:1375925.

### Example 2: ZIP12 Agonist Compounds

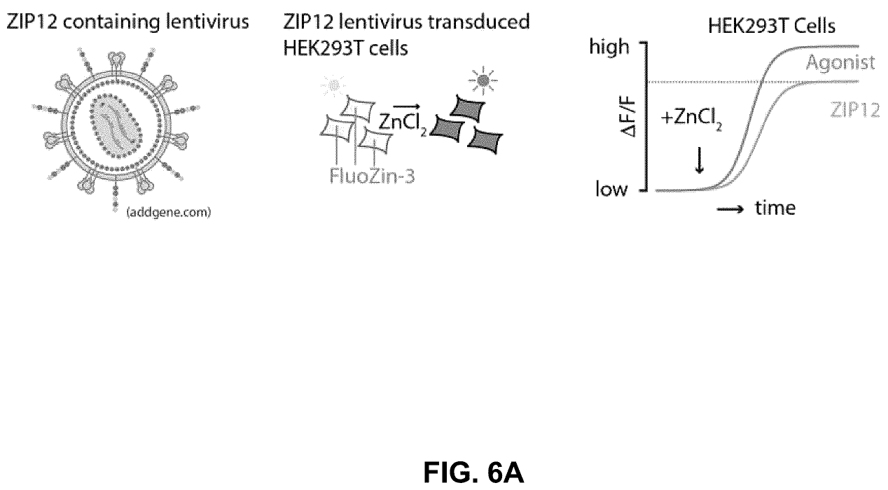

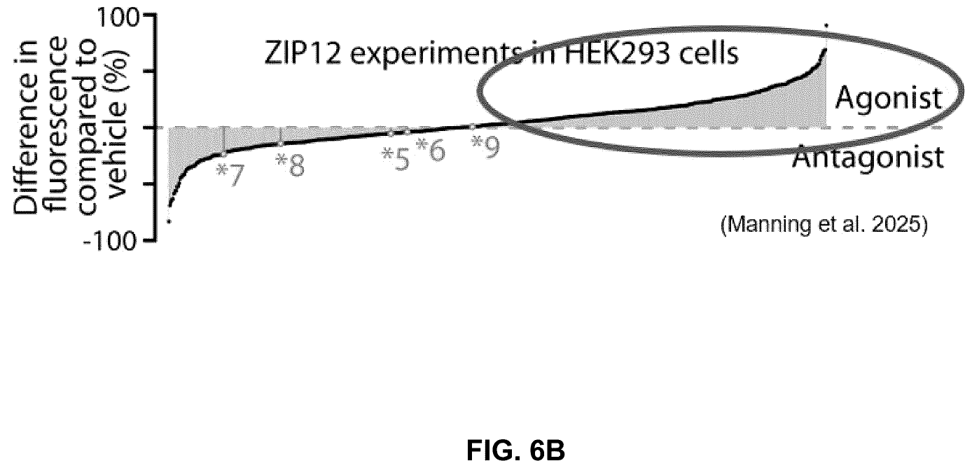

FIGS. 6A-6B: FIG. 6A shows an example experimental protocol to study ZIP12 agonists according to the present disclosure. FIG. 6B highlights the agonist activity of compounds from a hit screen described herein.

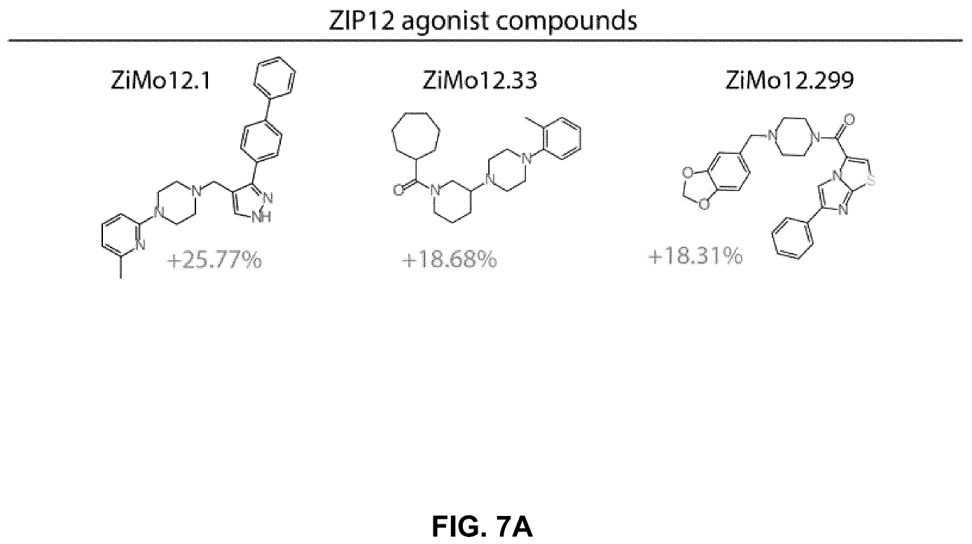

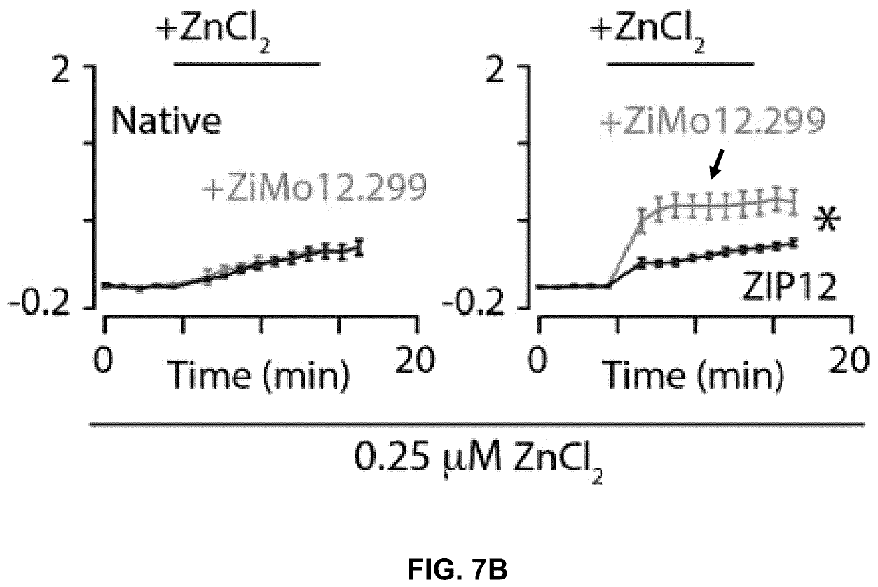

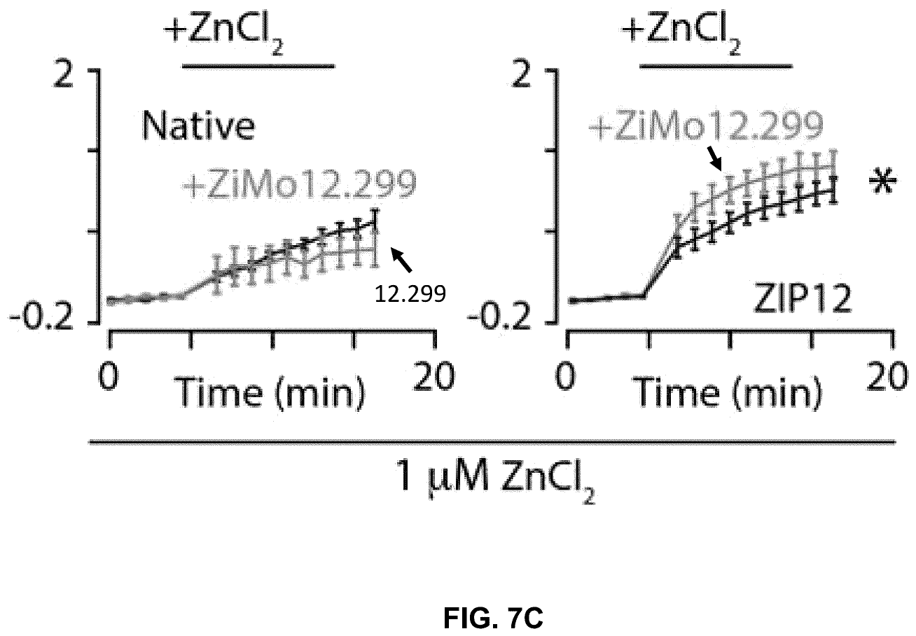

FIGS. 7A-7C: FIG. 7A displays representative examples of ZIP12 agonists according to the present disclosure. FIGS. 7B and 7C show activity of an example of FIG. 7A—ZiMo12.299.

As can be seen from FIGS. 7B and 7C, ZiMo12.299 is more effective with lower amounts of zinc.

### Example 3: Additional Examples of ZIP12 Modulators According to the Present Disclosure

Additional examples of ZIP12 modulators according to the present disclosure can be found in the table below. A ZIP12 agonist is denoted with a positive percentage value for “Difference in fluorescence compared to vehicle (%). Data shown in FIG. 3E” whereas a ZIP12 antagonist is denoted with a negative percentage value for “Difference in fluorescence compared to vehicle (%). Data shown in FIG. 3E.”

| | | Differe- |
|---|---|---|
| | | nce in |
| | | fluores- |
| | | cence |
| | | com- |
| | | pared to |
| | | vehicle |
| | | (%). |
| | | Data |
| | | shown |
| Compound | | in FIG. |
| Name | IUPAC Nomenclature | 3E |
| ZiMo12.586 | 1-[1-(2-chlorobenzoyl)-3-piperidinyl]- | 90.39 |
| | 4-(2-fluorophenyl)piperazine | |
| ZiMo12.362 | 2-methyl-6-({[2-(1-pyrrolidinyl- | 68.84 |
| | carbonyl)imidazo[1,2-a]pyridin-3- | |
| | yl]methyl}amino)-2-heptanol | |
| ZiMo12.157 | (4-chloro-2-methylphenyl)[1-(1H-pyrazol- | 68.39 |
| | 3-ylmethyl)-3-piperidinyl]methanone | |
| ZiMo12.493 | N-[(1-ethyl-2-pyrrolidinyl)methyl]- | 66.73 |
| | N-{[1-(2-methoxyethyl)- | |
| | 4-piperidinyl]methyl}- | |
| | 2-thiophenecarboxamide | |
| ZiMo12.263 | N-(1-{1-[(2,6-dimethyl-4- | 64.42 |
| | pyrimidinyl)carbonyl]-4-piperidinyl}-1H- | |
| | pyrazol-5-yl)-4-methoxybenzamide | |
| ZiMo12.313 | 2-butyl-N-[(4-methyl-5,6,7,8- | 63.22 |
| | tetrahydro-2-quinazolinyl)methyl]-1,3- | |
| | benzoxazole-5-carboxamide | |
| ZiMo12.541 | N-methyl-N-(1H-pyrazol-5-ylmethyl)-6-[4- | 59.96 |
| | (trifluoromethyl)phenyl]imidazo[2,1- | |
| | b][1,3]thiazole-3-carboxamide | |
| ZiMo12.487 | methyl 4-{[2-({[4-(1H-tetrazol- | 56.41 |
| | 1-yl)phenyl]amino}carbonyl)-1- | |
| | piperidinyl]methyl}benzoate | |
| ZiMo12.218 | 1-(1,3-benzodioxol-5-ylmethyl)- | 56.09 |
| | 4-{1-[(methylthio)acetyl]-3- | |
| | piperidinyl}piperazine | |
| ZiMo12.244 | 1-acetyl-4-(4-chloro-2- | 54.81 |
| | {[4-(methoxymethyl)-1- | |
| | piperidinyl]carbonyl}phenoxy)piperidine | |
| ZiMo12.118 | N-[(2-{methyl[2-(2-pyridinyl)ethyl]amino}-3- | 53.39 |
| | pyridinyl)methyl]imidazo[1,2- | |
| | a]pyridine-2-carboxamide | |
| ZiMo12.222 | 2-[(1-acetyl-4-piperidinyl)oxy]-5-chloro- | 53.18 |
| | N-[1-(4-fluorophenyl)ethyl]benzamide | |
| ZiMo12.348 | 2-ethoxy-6-{[3-(2-hydroxyethyl)- | 52.58 |
| | 4-(3-methoxybenzyl)-1- | |
| | piperazinyl]methyl}phenol | |
| ZiMo12.516 | N-{[1-(2,3-dihydro-1H-inden-2- | 52.52 |
| | yl)-3-piperidinyl]methyl}-N-[(4- | |
| | isopropyl-1,3-thiazol-2-yl)methyl]- | |
| | 2-methoxyethanamine | |
| ZiMo12.610 | N-(1-{1-[4-(acetylamino)benzyl]- | 51.07 |
| | 4-piperidinyl}-1H-pyrazol-5- | |
| | yl) cyclopentanecarboxamide | |
| ZiMo12.199 | 1-(1,3-benzodioxol-5-ylmethyl)-4-[1- | 49.83 |
| | (cyclopropylacetyl)-3-piperidinyl]piperazine | |
| ZiMo12.11 | N-(3-hydroxy-2,2-dimethylpropyl)- | 49.01 |
| | 5-{[(6-methyl-3-pyridinyl)oxy]methyl}- | |
| | 3-isoxazolecarboxamide | |
| ZiMo12.319 | 1′-[(4-methylphenoxy)acetyl]-N- | 48.18 |
| | (tetrahydro-2-furanylmethyl)-1,4′- | |
| | bipiperidine-4-carboxamide | |
| ZiMo12.351 | N-[1-(1-{[5-(hydroxymethyl)-2- | 47.98 |
| | furyl]methyl}-4-piperidinyl)-1H-pyrazol- | |
| | 5-yl]-3-methylbutanamide | |
| ZiMo12.559 | ethyl 1-{[(1,1-dioxidotetrahydro-3- | 47.86 |
| | thienyl)amino]carbonyl}-4-(2- | |
| | phenoxyethyl)-4-piperidinecarboxylate | |
| ZiMo12.50 | 1-{[3-(3-chlorophenyl)-1,2,4- | 46.09 |
| | oxadiazol-5-yl]methyl}-4-(2- | |
| | methoxyethoxy)piperidine | |
| ZiMo12.103 | 5-[(6,7-dimethoxy-3,4-dihydro-2(1H)- | 45.91 |
| | isoquinolinyl)carbonyl]-1-[2-(4- | |
| | morpholinyl)ethyl]-2-piperidinone | |
| ZiMo12.411 | N-[1-(2-fluorobenzyl)-3-piperidinyl]- | 45.89 |
| | 1-methyl-6-oxo-1,4,5,6- | |
| | tetrahydro-3-pyridazinecarboxamide | |
| ZiMo12.166 | (3R*,4R*)-4-[4-(2-fluorophenyl)-1- | 45.33 |
| | piperazinyl]-1-[(2E)-3-(3-pyridinyl)- | |
| | 2-propenoyl]-3-piperidinol | |
| ZiMo12.127 | 1-(2-fluorophenyl)-4-[1-(4- | 44.56 |
| | methoxy-3-methylbenzoyl)-3- | |
| | piperidinyl]piperazine | |
| ZiMo12.74 | N-(2,4-dimethoxybenzyl)-3-[1-(2- | 44.07 |
| | hydroxybenzyl)-3-piperidinyl]propanamide | |
| ZiMo12.504 | 1-{[3-(4-biphenylyl)-1H-pyrazol-4- | 43.93 |
| | yl]methyl}-3-(methoxymethyl)piperidine | |
| ZiMo12.71 | N-(2,1,3-benzoxadiazol-5- | 43.91 |
| | ylmethyl)-N-methyl-2-[3-oxo-1-(2- | |
| | pyridinylmethyl)-2-piperazinyl]acetamide | |
| ZiMo12.312 | N-{[3-(2,3-dihydro-1,4-benzodioxin- | 43.86 |
| | 6-yl)-1-methyl-1H-pyrazol-4- | |
| | yl]methyl}-2-(2-thienyl)ethanamine | |
| ZiMo12.32 | 1-(3-chlorophenyl)-4-[1-(4-pentenoyl)- | 43.02 |
| | 3-piperidinyl]piperazine | |
| ZiMo12.193 | N-(cyclohexylmethyl)-2-[1-(4- | 42.88 |
| | methoxy-3-methylbenzyl)-3-oxo-2- | |
| | piperazinyl]acetamide | |
| ZiMo12.443 | methyl 4-[(3-chloro-4-{[1-(methylsulfonyl)-4- | 42.78 |
| | piperidinyl]oxy}benzoyl)amino]butanoate | |
| ZiMo12.526 | 1-({2-[(4-benzyl-1-piperazinyl)methyl]- | 42.23 |
| | 1,3-oxazol-4-yl}carbonyl)-3-piperidinol | |
| ZiMo12.367 | ethyl 3-(2-fluorobenzyl)-1-[3-(2-oxo- | 41.62 |
| | 1-pyrrolidinyl)propanoyl]-3- | |
| | piperidinecarboxylate | |
| ZiMo12.338 | (3-fluorobenzyl){1-[1-(3-fluorophenyl)- | 41.38 |
| | 5-methyl-1H-pyrazol-4-yl]ethyl}amine | |
| ZiMo12.126 | N-(2,4-dimethoxybenzyl)-3-[1-(2- | 41.08 |
| | pyridinylmethyl)-3-piperidinyl]propanamide | |
| ZiMo12.140 | 2-{[1-(4-chloro-3-fluorobenzyl)- | 40.03 |
| | 4-piperidinyl]oxy}-4-methoxy-N-(2- | |
| | methoxyethyl)benzamide | |
| ZiMo12.27 | N-(2,4-dimethoxybenzyl)-3-[1- | 39.09 |
| | (4-hydroxy-3-methoxybenzyl)-3- | |
| | piperidinyl]propanamide | |
| ZiMo12.489 | 2-{[4-(2-fluorophenyl)-1- | 38.54 |
| | piperazinyl]methyl}-N-(2-methoxy-1- | |
| | methylethyl)-1,3-oxazole-4-carboxamide | |
| ZiMo12.485 | N-{[1-(3-isoxazolylmethyl)-3- | 38.44 |
| | piperidinyl]methyl}-4-biphenylcarboxamide | |
| ZiMo12.165 | 1-[1-(3-cyclopentylpropanoyl)-3- | 38.28 |
| | piperidinyl]-4-(2-methylphenyl)piperazine | |
| ZiMo12.535 | 2,6-dimethoxy-N-[1-(2-phenylethyl)- | 38.23 |
| | 3-piperidinyl]nicotinamide | |
| ZiMo12.332 | 6-[(3-anilino-1-piperidinyl)carbonyl]- | 38.11 |
| | 2,2-dimethyl-2,3-dihydro-4H- | |
| | pyran-4-one | |
| ZiMo12.571 | 2-[(1-ethyl-1H-pyrazol-4-yl)carbonyl]-7- | 37.73 |
| | (3-methoxybenzyl)-2,7-diazaspiro[4.5]decane | |
| ZiMo12.231 | 2-(4-morpholinyl)-N-[1-(2-phenylethyl)- | 37.68 |
| | 3-piperidinyl]benzamide | |
| ZiMo12.507 | 1-(2-{3-[4-(4-methoxyphenyl)-1- | 37.67 |
| | piperazinyl]-1-piperidinyl}-2- | |
| | oxoethyl)azepane | |
| ZiMo12.320 | methyl (2S*,4S*,5R*)-5-(2,3- | 37.53 |
| | difluorophenyl)-1-methyl-4-{[(3- | |
| | pyridinylmethyl)amino]carbonyl}- | |
| | 2-pyrrolidinecarboxylate | |
| ZiMo12.101 | methyl 4-[7-(2,3-difluorobenzyl)-2,7- | 37.14 |
| | diazaspiro[4.5]dec-2-yl]-4-oxobutanoate | |
| ZiMo12.330 | 1-[1-(3-butenoyl)-3-piperidinyl]-4-[3- | 37.07 |
| | (trifluoromethyl)phenyl]piperazine | |
| ZiMo12.494 | methyl (2S*,4S*,5R*)-4-{[(trans-4- | 36.75 |
| | hydroxycyclohexyl)amino]carbonyl}- | |
| | 1,2-dimethyl-5-(2-methylphenyl)- | |
| | 2-pyrrolidinecarboxylate | |
| ZiMo12.205 | 4-{[1-(cyclopropylcarbonyl)-4- | 36.61 |
| | piperidinyl]oxy}-N-[2-(3,5-dimethyl-1H- | |
| | pyrazol-1-yl)-1-methylethyl]benzamide | |
| ZiMo12.298 | (3S*)-1-ethyl-4-{[3-(6-methoxy- | 36.09 |
| | 2-naphthyl)-1H-pyrazol-4- | |
| | yl]methyl}-3-methyl-2-piperazinone | |
| ZiMo12.78 | 2-[(1-acetyl-4-piperidinyl)oxy]-5-chloro-N- | 36.06 |
| | (3-fluoro-4-methylbenzyl)benzamide | |
| ZiMo12.180 | N-(2-methoxyphenyl)-N′-(1- | 35.53 |
| | {1-[3-(4-pyridinyl)propanoyl]-4- | |
| | piperidinyl}-1H-pyrazol-5-yl)urea | |
| ZiMo12.512 | 2-[(1-acetyl-4-piperidinyl)oxy]-5-methoxy- | 35.38 |
| | N-methyl-N-(tetrahydro-2H-pyran- | |
| | 2-ylmethyl)benzamide | |
| ZiMo12.345 | 6-(3-fluorophenyl)-3-[(4-methoxy-1- | 34.3 |
| | piperidinyl)carbonyl]imidazo[2,1- | |
| | b][1,3]thiazole | |
| ZiMo12.251 | 1-[(6-fluoro-1H-benzimidazol-2-yl)methyl]- | 34 |
| | N-methyl-N-(2-phenylethyl)-3-piperidinamine | |
| ZiMo12.100 | N′-cyclopentyl-N-ethyl-N- | 33.69 |
| | methyl[1,2,5]oxadiazolo[3,4-b]pyrazine-5,6- | |
| | diamine | |
| ZiMo12.145 | (3-furylmethyl){[1-(2-methylbenzyl)- | 33.49 |
| | 4-piperidinyl]methyl}(tetrahydro- | |
| | 2-furanylmethyl)amine | |
| ZiMo12.300 | 2-[(1-acetyl-4-piperidinyl)oxy]-5-chloro- | 33.48 |
| | N-[(6-methyl-2-pyridinyl)methyl]benzamide | |
| ZiMo12.578 | 4-methoxy-N-(2-methoxyethyl)- | 33.12 |
| | 2-{[1-(4,4,4-trifluorobutyl)-4- | |
| | piperidinyl]oxy}benzamide | |
| ZiMo12.260 | N-({1-[(3-isobutyl-1H-pyrazol-5-yl)carbonyl]- | 32.91 |
| | 3-piperidinyl}methyl)-1- | |
| | phenylmethanesulfonamide | |
| ZiMo12.63 | 4-methoxy-2-{[7-(2-phenylethyl)-2,7- | 32.12 |
| | diazaspiro[4.5]dec-2-yl]methyl}phenol | |
| ZiMo12.147 | 1-[1-(1-cyclopenten-1-ylcarbonyl)-3- | 31.52 |
| | piperidinyl]-4-(2-methylphenyl)piperazine | |
| ZiMo12.133 | 2-{[7-(3-methoxybenzyl)-2,7- | 31.43 |
| | diazaspiro[4.5]dec-2-yl]carbonyl}-4- | |
| | methylphenol | |
| ZiMo12.99 | 6-(2-chlorophenyl)-N-isopropyl-N-[(1- | 31.37 |
| | methyl-1H-pyrazol-4-yl)methyl]imidazo[2,1- | |
| | b][1,3]thiazole-3-carboxamide | |
| ZiMo12.95 | N-methyl-1-(2-phenylethyl)-N-[(2-propyl- | 31.28 |
| | 5-pyrimidinyl)methyl]-3-piperidinamine | |
| ZiMo12.150 | N-(2,4-dimethoxybenzyl)-3-[1-(3- | 31.06 |
| | hydroxybenzyl)-3-piperidinyl]propanamide | |
| ZiMo12.384 | 1-{3-[5-(1,3-benzodioxol-5-yl)-1,3,4- | 30.84 |
| | oxadiazol-2-yl]propanoyl}-4- | |
| | (cyclopropylmethyl)piperazine | |
| ZiMo12.375 | N-(1-{1-[(6-ethoxy-2-quinolinyl)methyl]-4- | 30.46 |
| | piperidinyl}-1H-pyrazol-5-yl)acetamide | |
| ZiMo12.587 | methyl (2S*,4S*,5R*)-5-(2,3-difluorophenyl)- | 30.27 |
| | 1-methyl-4-{[(4- | |
| | pyridinylmethyl)amino]carbonyl}- | |
| | 2-pyrrolidinecarboxylate | |
| ZiMo12.565 | methyl 3-{[3-(hydroxymethyl)-3-(2- | 30.03 |
| | phenoxyethyl)-1- | |
| | piperidinyl]carbonyl}benzoate | |
| ZiMo12.238 | 1-(cyclopentylmethyl)-4-[2-(1- | 29.87 |
| | piperidinylcarbonyl)phenoxy]piperidine | |
| ZiMo12.460 | N-[(2-{methyl[2-(2-pyridinyl)ethyl]amino}- | 29.46 |
| | 3-pyridinyl)methyl]-3-furamide | |
| ZiMo12.591 | 5-(4-fluorophenyl)-N-methyl-N-{[1-(4- | 28.93 |
| | morpholinyl)cyclohexyl]methyl}- | |
| | 1,2,4-triazin-3-amine | |
| ZiMo12.457 | 1-{[2-(benzylsulfonyl)-1-isobutyl-1H- | 28.58 |
| | imidazol-5-yl]methyl}-3-methoxypiperidine | |
| ZiMo12.34 | 6-oxo-N-[(2-phenyl-1,3-oxazol-4-yl)methyl]- | 28.48 |
| | 1-(2-pyridinylmethyl)-3- | |
| | piperidinecarboxamide | |
| ZiMo12.311 | 1-ethyl-N-{[1-(2-fluorobenzyl)-4- | 28.45 |
| | piperidinyl]methyl}-3-methyl-N-(tetrahydro-2- | |
| | furanylmethyl)-1H-pyrazole-5-carboxamide | |
| ZiMo12.198 | N-methyl-2-[3-oxo-1-(3-phenylpropyl)-2- | 28.22 |
| | piperazinyl]-N-(3-pyridinylmethyl)acetamide | |
| ZiMo12.237 | 4-(3-methoxypropyl)-1-{[3-(1-naphthyl)- | 28.08 |
| | 1H-pyrazol-4-yl]methyl}piperidine | |
| ZiMo12.409 | (3R*,4R*)-1-(3-chloro-4,5-dimethoxybenzyl)- | 27.98 |
| | 4-(4-methyl-1-piperazinyl)-3-piperidinol | |
| ZiMo12.502 | (3aS*,5S*,9aS*)-2-(3- | 27.95 |
| | chlorobenzyl)-5-(3-ethoxy-4- | |
| | hydroxyphenyl)hexahydro-7H-pyrrolo[3,4- | |
| | g]pyrrolizin-1(2H)-one | |
| ZiMo12.38 | 1′-cyclohexyl-N-(3-pyridinylmethyl)- | 27.87 |
| | 1,4′-bipiperidine-4-carboxamide | |
| ZiMo12.561 | 2-{[4-(4-ethoxybenzyl)-3-(2-hydroxyethyl)- | 27.62 |
| | 1-piperazinyl]methyl}-6-methoxyphenol | |
| ZiMo12.200 | N-methyl-2-{3-oxo-1-[3- | 27.44 |
| | (trifluoromethyl)benzyl]-2-piperazinyl}-N-(2- | |
| | phenylethyl)acetamide | |
| ZiMo12.177 | 5-[(4-chloro-3,5-dimethyl- | 27.36 |
| | phenoxy)methyl]-N-[(5-methyl-2- | |
| | pyrazinyl)methyl]-3-isoxazolecarboxamide | |
| ZiMo12.530 | N-(6,6-dimethyl-1-phenyl-4,5,6,7- | 27.27 |
| | tetrahydro-1H-indazol-4-yl)-3-(4- | |
| | pyridinyl)propanamide | |
| ZiMo12.160 | 1-cyclobutyl-4-[2-(1-piperidinyl- | 26.68 |
| | carbonyl)phenoxy]piperidine | |
| ZiMo12.304 | 1-[3-(4-biphenylyl)-1H-pyrazol- | 26.64 |
| | 4-yl]-N-[(1-ethyl-1H-pyrazol-4- | |
| | yl)methyl]methanamine | |
| ZiMo12.382 | 6-phenyl-3-{[4-(1-pyrrolidinyl)-1- | 26.5 |
| | piperidinyl]carbonyl}imidazo[2,1- | |
| | b][1,3]thiazole | |
| ZiMo12.45 | N-[2-({3-[methyl(2-phenylethyl)amino]-1- | 26.49 |
| | piperidinyl}methyl)-6-quinolinyl]acetamide | |
| ZiMo12.315 | 2-[1-(3-{5-[2-(2-methoxyphenyl)ethyl]- | 26.42 |
| | 1,3,4-oxadiazol-2-yl}propanoyl)- | |
| | 2-piperidinyl]ethanol | |
| ZiMo12.307 | N-(2,4-dimethoxybenzyl)-3-[1- | 26.29 |
| | (2-furylmethyl)-3-piperidinyl]propanamide | |
| ZiMo12.21 | 1,3-benzodioxol-5-yl(1-{[2-(2-thienyl)- | 26.26 |
| | 1,3-thiazol-4-yl]carbonyl}-3- | |
| | piperidinyl)methanone | |
| ZiMo12.490 | [5-({4-[5-chloro-2-(1- | 26.06 |
| | piperidinylcarbonyl)phenoxy]-1- | |
| | piperidinyl}methyl)-2-furyl]methanol | |
| ZiMo12.403 | 1′-(3-ethynylbenzoyl)-N-(tetrahydro-2- | 25.89 |
| | furanylmethyl)-1,4′-bipiperidine- | |
| | 4-carboxamide | |
| ZiMo12.1 | 1-{[3-(4-biphenylyl)-1H- | 25.77 |
| | pyrazol-4-yl]methyl}- | |
| | 4-(1H-1,2,4-triazol-3-ylcarbonyl)piperazine | |
| ZiMo12.608 | 1-(3-butenoyl)-N-(3′-methyl-4-biphenylyl)- | 25.65 |
| | 4-piperidinecarboxamide | |
| ZiMo12.463 | N-benzyl-3-{[(2,4-difluoro- | 25.6 |
| | benzyl)amino]methyl}- | |
| | N-methylimidazo[1,2- | |
| | alpyridine-2-carboxamide | |
| ZiMo12.60 | 4-[1-({3-[4-(trifluoromethyl)phenyl]- | 25.56 |
| | 1,2,4-oxadiazol-5-yl}methyl)-4- | |
| | piperidinyl]thiomorpholine | |
| ZiMo12.359 | 1-(1-{[2-(isopropylsulfonyl)-1- | 25.39 |
| | (2-phenylethyl)-1H-imidazol-5- | |
| | yl]methyl}-3-piperidinyl)ethanone | |
| ZiMo12.239 | 3-(dimethylamino)-N-(1′-methyl- | 25.17 |
| | 1,4′-bipiperidin-3-yl)benzamide | |
| ZiMo12.431 | 3-chloro-N-cyclohexyl-4- | 25.12 |
| | {[1-(methylsulfonyl)- | |
| | 4-piperidinyl]oxy}benzamide | |
| ZiMo12.19 | N-[3-(3,5-dimethyl-1H-pyrazol-1- | 25.02 |
| | yl)benzyl]-N-methyl-6-oxo-1-[2-(2- | |
| | pyridinyl)ethyl]-3-piperidinecarboxamide | |
| ZiMo12.303 | N-[3-(1H-tetrazol-1-yl)phenyl]-1- | 25 |
| | [(3,5,6-trimethyl-2-pyrazinyl)methyl]- | |
| | 3-piperidinecarboxamide | |
| ZiMo12.604 | N-(2,4-dimethoxybenzyl)-3-[1-(4- | 24.67 |
| | hydroxybenzyl)-3-piperidinyl]propanamide | |
| ZiMo12.536 | 2-(4-fluorophenyl)-1-{[3-(4- | 24.52 |
| | methoxyphenyl)-1,2,4-oxadiazol-5- | |
| | yl]methyl}piperidine | |
| ZiMo12.14 | 3-chloro-4-{[1-(methylsulfonyl)-4- | 24.51 |
| | piperidinyl]oxy}-N-[2-(1H-pyrazol-1- | |
| | yl)ethyl]benzamide | |
| ZiMo12.522 | 1-(1-{[3-(2-chlorobenzyl)-1,2,4- | 24.34 |
| | oxadiazol-5-yl]methyl}-3-piperidinyl)- | |
| | 3-methyl-1-butanone | |
| ZiMo12.17 | 2-{[4-(2-ethoxybenzyl)-3-(2- | 23.46 |
| | hydroxyethyl)-1-piperazinyl]methyl}-6- | |
| | methoxyphenol | |
| ZiMo12.603 | N-[(2-ethyl-1,3-thiazol-4-yl)methyl]- | 23.07 |
| | 1-[2-(4-morpholinyl)ethyl]-6-oxo- | |
| | 3-piperidinecarboxamide | |
| ZiMo12.391 | N-(3-methoxypropyl)-6-[4- | 22.96 |
| | (trifluoromethyl)phenyl]imidazo[2,1- | |
| | b][1,3]thiazole-3-carboxamide | |
| ZiMo12.75 | N-ethyl-N-({1-[2-(2-methylphenyl)ethyl]- | 22.89 |
| | 4-piperidinyl}methyl)-1- | |
| | cyclopentene-1-carboxamide | |
| ZiMo12.80 | N-(2-hydroxy-1,1-dimethylethyl)-6-[4- | 22.73 |
| | (trifluoromethyl)phenyl]imidazo[2,1- | |
| | b][1,3]thiazole-3-carboxamide | |
| ZiMo12.52 | N-[(1-ethyl-3,5-dimethyl-1H-pyrazol- | 22.69 |
| | 4-yl)methyl]-4-{[1-(methoxyacetyl)- | |
| | 4-piperidinyl]oxy}benzamide | |
| ZiMo12.236 | ethyl 1-[(3-methyl-5,6-dihydro- | 22.55 |
| | 1,4-dioxin-2-yl)carbonyl]-4-[2- | |
| | (trifluoromethyl)benzyl]-4- | |
| | piperidinecarboxylate | |
| ZiMo12.182 | N-(tert-butyl)-5-chloro-2-{[1- | 22.5 |
| | (tetrahydro-2H-thiopyran-4-yl)-4- | |
| | piperidinyl]oxy}benzamide | |
| ZiMo12.148 | 5-methyl-N-[(1-{[1-(3-methylphenyl)- | 22.47 |
| | 1H-imidazol-2-yl]methyl}-3- | |
| | piperidinyl)methyl]-3-isoxazolecarboxamide | |
| ZiMo12.607 | 5-[(4-chloro-2-methoxyphenoxy)methyl]- | 22.39 |
| | N-[(1-methyl-1H-pyrazol-4- | |
| | yl)methyl]-3-isoxazolecarboxamide | |
| ZiMo12.290 | methyl 4-({4-[4-(1- | 22.36 |
| | pyrrolidinylcarbonyl)phenoxy]-1- | |
| | piperidinyl}methyl)benzoate | |
| ZiMo12.363 | 5-(1H-benzimidazol-1-ylmethyl)- | 22.24 |
| | N-methyl-N-[(3-methyl-4-pyridinyl)methyl]- | |
| | 1H-pyrazole-3-carboxamide | |
| ZiMo12.248 | N-{1-[1-(1H-indol-3-ylmethyl)- | 22.04 |
| | 4-piperidinyl]-1H-pyrazol-5- | |
| | yl}cyclopentanecarboxamide | |
| ZiMo12.259 | 1′-[(5-cyclopentyl-2-thienyl)methyl]- | 21.98 |
| | N-cyclopropyl-1,4′-bipiperidine-4- | |
| | carboxamide | |
| ZiMo12.112 | N-[1-(2-phenylethyl)-3- | 21.89 |
| | piperidinyl]nicotinamide | |
| ZiMo12.585 | 1-isopropyl-4-{[2-(isopropylsulfonyl)- | 21.81 |
| | 1-(3-methylbutyl)-1H-imidazol-5- | |
| | yl]methyl}piperazine | |
| ZiMo12.560 | N-[(6-chloro-1,3-benzodioxol-5- | 21.65 |
| | yl)methyl]-1-(5-methyl-1-phenyl-1H- | |
| | pyrazol-4-yl)ethanamine | |
| ZiMo12.258 | 2-(2-{3-[4-(2-methylphenyl)-1- | 20.97 |
| | piperazinyl]-1-piperidinyl}-2-oxoethyl)- | |
| | 2H-1,2,3-benzotriazole | |
| ZiMo12.73 | (3aS*,6aR*)-3-[3-(4- | 20.89 |
| | methoxyphenyl)propyl]-5-[(4-methyl-1H- | |
| | imidazol-5-yl)methyl]hexahydro-2H- | |
| | pyrrolo[3,4-d][1,3]oxazol-2-one | |
| ZiMo12.189 | 1-(2-chlorobenzyl)-N-methyl-N-(2- | 20.8 |
| | phenylethyl)-1H-1,2,3-triazole-4- | |
| | carboxamide | |
| ZiMo12.174 | methyl (2S*,4S*,5R*)-1,2-dimethyl- | 20.71 |
| | 4-({methyl[(4-methyl-1H-imidazol- | |
| | 2-yl)methyl]amino}carbonyl)-5-(2- | |
| | methylphenyl)-2-pyrrolidinecarboxylate | |
| ZiMo12.164 | N,N-diallyl-2-[1-(3-fluorobenzyl)-3- | 20.5 |
| | oxo-2-piperazinyl]acetamide | |
| ZiMo12.347 | N-[1-(3-fluorophenyl)-6,6-dimethyl- | 19.75 |
| | 4,5,6,7-tetrahydro-1H-indazol-4- | |
| | yl]-2-(2-oxo-1-pyrrolidinyl)acetamide | |
| ZiMo12.210 | N-(1-{1-[2-(1H-pyrazol-1-yl)acetyl]- | 19.59 |
| | 4-piperidinyl}-1H-pyrazol-5- | |
| | yl)cyclopentanecarboxamide | |
| ZiMo12.401 | ethyl 1-benzoyl-3-(4-fluorobenzyl)- | 19.58 |
| | 3-piperidinecarboxylate | |
| ZiMo12.569 | 3-chloro-N-methyl-N-[(4-methyl- | 19.39 |
| | 1H-imidazol-2-yl)methyl]-4-{[1- | |
| | (methylsulfonyl)-4- | |
| | piperidinyl]oxy}benzamide | |
| ZiMo12.589 | 1′-[3-(1-methylcyclopropyl)propanoyl]- | 19.38 |
| | N-(tetrahydro-2-furanylmethyl)- | |
| | 1,4′-bipiperidine-4-carboxamide | |
| ZiMo12.151 | 4-({1-butyl-2-[(4- | 19.26 |
| | methylpentyl)sulfonyl]-1H-imidazol-5- | |
| | yl}methyl)morpholine | |
| ZiMo12.448 | 6-({1-[4-(methylthio)phenyl]-1,3,4,9- | 19.26 |
| | tetrahydro-2H-beta-carbolin-2- | |
| | yl}carbonyl)-2,4(1H,3H)-pyrimidinedione | |
| ZiMo12.492 | N-ethyl-6-(3-fluorophenyl)-N-(2- | 19.2 |
| | methyl-2-propen-1-yl)imidazo[2,1- | |
| | b][1,3]thiazole-3-carboxamide | |
| ZiMo12.181 | 3-chloro-N-[(6-methyl-2- | 19.14 |
| | pyridinyl)methyl]-4-{[1-(methylsulfonyl)-4- | |
| | piperidinyl]oxy}benzamide | |
| ZiMo12.97 | ethyl 3-(2-fluorobenzyl)-1-(3- | 19.09 |
| | pyridinylcarbonyl)-3-piperidinecarboxylate | |
| ZiMo12.68 | 2-{[4-(2-ethoxybenzyl)-3-(2-hydroxyethyl)- | 19.06 |
| | 1-piperazinyl]methyl}-5- | |
| | methoxyphenol | |
| ZiMo12.33 | 3-{[4-(1,3-benzodioxol-5-ylmethyl)- | 18.68 |
| | 1-piperazinyl]carbonyl}-6- | |
| | phenylimidazo[2,1-b][1,3]thiazole | |
| ZiMo12.57 | 4-{3-[(2-methoxyethyl)amino]-3- | 18.67 |
| | oxopropyl}-N-(2-methoxy-5- | |
| | methylphenyl)-1-piperidinecarboxamide | |
| ZiMo12.39 | ethyl 1-[3-(5-fluoro-1H-benzimidazol- | 18.64 |
| | 2-yl)propanoyl]-4-(tetrahydro- | |
| | 2H-pyran-2-ylmethyl)-4-piperidinecarboxylate | |
| ZiMo12.337 | methyl 4-[{{1-[2-(4-methoxyphenyl)ethyl]-4- | 18.38 |
| | piperidinyl}methyl)(methyl)amino]- | |
| | 4-oxobutanoate | |
| ZiMo12.299 | 1-[1-(cycloheptylcarbonyl)-3-piperidinyl]- | 18.31 |
| | 4-(2-methylphenyl)piperazine | |
| ZiMo12.77 | 2-[4-(3,4-difluorobenzyl)-1-(4-methoxy- | 18.22 |
| | 3-methylbenzyl)-2-piperazinyl]ethanol | |
| ZiMo12.285 | 1-(2-ethyl-4-methyl-1H-imidazol-5-yl)-N- | 18.16 |
| | methyl-N-({1-[2-(2-methylphenyl)ethyl]- | |
| | 3-piperidinyl}methyl)methanamine | |
| ZiMo12.352 | N-(1,1-dioxidotetrahydro-3-thienyl)-2-(3- | 17.85 |
| | phenylpropyl)-1,3-benzoxazole-5-carboxamide | |
| ZiMo12.49 | N-methyl-1-(2-phenylethyl)-N- | 17.81 |
| | [(3,5,6-trimethyl-2-pyrazinyl)methyl]-3- | |
| | piperidinamine | |
| ZiMo12.246 | 1-[2-(3-fluorophenyl)ethyl]-6-oxo-N-(2,2,2- | 17.77 |
| | trifluoroethyl)-3-piperidinecarboxamide | |
| ZiMo12.427 | 1-{5-[(4-isopropyl-1-piperazinyl)methyl]- | 17.67 |
| | 2-methoxyphenoxy}-3-(4- | |
| | methyl-1-piperazinyl)-2-propanol | |
| ZiMo12.106 | 5-chloro-4-{[3-(2,3-dihydro- | 17.57 |
| | 1,4-benzodioxin-6- | |
| | ylamino)-1-piperidinyl]methyl}- | |
| | 2-methoxyphenol | |
| ZiMo12.265 | N-(2-hydroxy-1,1-dimethylethyl)-2- | 17.41 |
| | {3-oxo-1-[3-(trifluoromethyl)benzyl]- | |
| | 2-piperazinyl}acetamide | |
| ZiMo12.25 | 6-(4-chlorophenyl)-3-[(4-isopropyl-1- | 17.39 |
| | piperazinyl)carbonyl]imidazo[2,1- | |
| | b][1,3]thiazole | |
| ZiMo12.241 | 1-isopropyl-2-{[1-(3-methoxypropyl)- | 17.32 |
| | 2-(methylsulfonyl)-1H-imidazol-5- | |
| | yl]methyl}-2,3,4,9-tetrahydro- | |
| | 1H-beta-carboline | |
| ZiMo12.317 | N-[(2-phenoxy-3-pyridinyl)methyl]tetrahydro- | 17.29 |
| | 3-furancarboxamide | |
| ZiMo12.531 | N-cyclobutyl-1-(2,3-dimethylphenyl)- | 17.25 |
| | 4,5,6,7-tetrahydro-1H-indazol-4- | |
| | amine | |
| ZiMo12.44 | 3-[1-(5-chloro-2-hydroxybenzyl)- | 17.11 |
| | 3-piperidinyl]-N-(2,4- | |
| | dimethoxybenzyl)propanamide | |
| ZiMo12.495 | N-{[1-butyl-2-(cyclohexylsulfonyl)- | 17.07 |
| | 1H-imidazol-5-yl]methyl}-N-methyl- | |
| | 2-(1H-pyrazol-4-yl)ethanamine | |
| ZiMo12.482 | N-(4-methoxy-2-methylphenyl)- | 16.83 |
| | 3-[1-(4,4,4-trifluorobutyl)-3- | |
| | piperidinyl]propanamide | |
| ZiMo12.414 | N~2 ~-acetyl-N~1~-{[1-(2- | 16.38 |
| | methylbenzyl)-4-piperidinyl]methyl}-N~1~- | |
| | (tetrahydro-2-furanylmethyl)glycinamide | |
| ZiMo12.539 | N-{[2-(2-methoxyethyl)-5- | 16.18 |
| | pyrimidinyl]methyl}-N-methyl-1-(2- | |
| | phenylethyl)-3-piperidinamine | |
| ZiMo12.445 | 3-phenoxy-N-{1-[1-(1,3-thiazol-2- | 16.13 |
| | ylmethyl)-4-piperidinyl]-1H-pyrazol- | |
| | 5-yl}propanamide | |
| ZiMo12.197 | ethyl 3-(4-fluorobenzyl)-1-(4-pentenoyl)- | 16.03 |
| | 3-piperidinecarboxylate | |
| ZiMo12.519 | N-methyl-6-phenyl-N-[2-(tetrahydro- | 16.01 |
| | 2H-pyran-2-yl)ethyl]imidazo[2,1- | |
| | b][1,3]thiazole-3-carboxamide | |
| ZiMo12.24 | N-(2,4-dimethoxybenzyl)-3-[1-(4- | 15.98 |
| | methoxybenzyl)-3-piperidinyl]propanamide | |
| ZiMo12.12 | 2-[4-cyclopentyl-1-(4-methoxy-3- | 15.9 |
| | methylbenzyl)-2-piperazinyl]ethanol | |
| ZiMo12.102 | 1-[1-(3-fluorobenzoyl)-3-piperidinyl]- | 15.86 |
| | 4-(2-methylphenyl)piperazine | |
| ZiMo12.228 | N-[3-(2-furyl)phenyl]-1-[(1-methyl-1H- | 15.79 |
| | pyrrol-3-yl)acetyl]-4-piperidinecarboxamide | |
| ZiMo12.595 | N-(3-isoxazolylmethyl)-6-[4- | 15.79 |
| | (trifluoromethyl)phenyl]imidazo[2,1- | |
| | b][1,3]thiazole-3-carboxamide | |
| ZiMo12.81 | 1-(3,5-dimethylphenyl)-N- | 15.32 |
| | (tetrahydro-2H-pyran-4-yl)-4,5,6,7- | |
| | tetrahydro-1H-indazol-4-amine | |
| ZiMo12.184 | ethyl 3-(2-fluorobenzyl)-1- | 15.32 |
| | (6-quinoxalinylcarbonyl)-3- | |
| | piperidinecarboxylate | |
| ZiMo12.234 | N-({1-[2-(4-methoxyphenyl)ethyl]- | 15.29 |
| | 4-piperidinyl}methyl)-N,3-dimethyl- | |
| | 5,6-dihydro-1,4-dioxine-2-carboxamide | |
| ZiMo12.250 | 3-(1-azocanylcarbonyl)-6-(3- | 15.27 |
| | chlorophenyl)imidazo[2,1-b][1,3]thiazole | |
| ZiMo12.322 | N-(1-{1-[(3-phenyl-1H-pyrazol-4- | 15.09 |
| | yl)methyl]-4-piperidinyl}-1H-pyrazol- | |
| | 5-yl)benzamide | |
| ZiMo12.327 | 4-(dimethylamino)-N-[1-(2-phenylethyl)- | 15.08 |
| | 3-piperidinyl]benzamide | |
| ZiMo12.163 | 4-{[{[3-(1-benzofuran-2-yl)-1-benzyl-1H- | 15.07 |
| | pyrazol-4-yl]methyl}(methyl)amino]methyl}- | |
| | 1-ethyl-2-pyrrolidinone | |
| ZiMo12.455 | 3-methyl-N-[1-(2-phenylethyl)-3- | 15.07 |
| | piperidinyl]-1-propyl-1H-pyrazole-4- | |
| | carboxamide | |
| ZiMo12.143 | methyl 4-({[(1-cycloheptyl-3-piperidinyl) | 15.06 |
| | amino]carbonyl}amino)benzoate | |
| ZiMo12.297 | 2-(1H-indazol-1-yl)-N-[1-(2-phenylethyl)- | 15.04 |
| | 3-piperidinyl]acetamide | |
| ZiMo12.314 | N-{1-[1-(3-ethoxybenzyl)-4-piperidinyl]- | 14.75 |
| | 1H-pyrazol-5-yl}cyclopentanecarboxamide | |
| ZiMo12.390 | N-{[3-(3,4-dimethylphenyl)-1H-pyrazol- | 14.65 |
| | 4-yl]methyl}-1-phenylcyclopropanamine | |
| ZiMo12.275 | (1-{[3-(3-chlorophenyl)-1-methyl-1H- | 14.46 |
| | pyrazol-4-yl]methyl}-4-piperidinyl)(3- | |
| | pyridinyl)methanol | |
| ZiMo12.446 | 2-({[2-(3,4-dihydro-2(1H)-isoquinolinyl)-3- | 14.46 |
| | pyridinyl]methyl}amino)nicotinamide | |
| ZiMo12.36 | N-[1-(2,3-dimethylphenyl)-4,5,6,7- | 14.4 |
| | tetrahydro-1H-indazol-4-yl]-2-(2- | |
| | oxo-1-pyrrolidinyl)acetamide | |
| ZiMo12.350 | N-(cyclopropylmethyl)-1′-(2-phenylethyl)- | 14.29 |
| | 1,4′-bipiperidine-3-carboxamide | |
| ZiMo12.43 | N-(4-isopropylphenyl)- | 14.28 |
| | 1-[(methylthio)acetyl]-3-piperidinamine | 14.2 |
| ZiMo12.294 | N-[(2,3-dimethyl-1H-indol-5- | |
| | yl)methyl]-1-(3-pyridinyl)-2-propanamine | |
| ZiMo12.26 | N-(1-cycloheptyl-3-piperidinyl)-1-methyl- | 14.18 |
| | 1H-pyrazole-5-carboxamide | |
| ZiMo12.342 | 2-chloro-4-[{{1-[1-(3-fluorophenyl)-5-methyl- | 14.14 |
| | 1H-pyrazol-4-yl]ethyl}amino)methyl]phenol | |
| ZiMo12.309 | N-{[1-(3-methoxyphenyl)-1H-pyrazol- | 14.12 |
| | 4-yl]methyl}-N,N′,N′- | |
| | trimethyl[1,2,5]oxadiazolo[3,4- | |
| | b]pyrazine-5,6-diamine | |
| ZiMo12.478 | N-[(5-methyl-2-thienyl)methyl]-5-[(8- | 14.08 |
| | quinolinyloxy)methyl]-3- | |
| | isoxazolecarboxamide | |
| ZiMo12.142 | 6-(1-azepanylcarbonyl)-2-(3,4- | 13.92 |
| | dimethoxybenzyl)-1,3-benzoxazole | |
| ZiMo12.374 | 1-(4-chlorophenyl)-N-({1-[3-(1H- | 13.91 |
| | pyrazol-1-yl)propyl]-3- | |
| | piperidinyl}methyl)cyclopropanecarboxamide | |
| ZiMo12.484 | ethyl 3-(3-chlorobenzyl)-1-[(3- | 13.8 |
| | methyl-5,6-dihydro-1,4-dioxin-2- | |
| | yl)carbonyl]-3-piperidinecarboxylate | |
| ZiMo12.291 | 2-(1-(2-fluorobenzyl)-4-{[1-(2- | 13.71 |
| | pyridinyl)-1H-pyrrol-2-yl]methyl}-2- | |
| | piperazinyl)ethanol | |
| ZiMo12.520 | 1′-(3-chloro-4-fluorobenzoyl)-N-isopropyl- | 13.56 |
| | 1,4′-bipiperidine-4-carboxamide | |
| ZiMo12.82 | ethyl 4-{methyl[1-(2-phenylethyl)-3- | 13.48 |
| | piperidinyl]amino}-1-piperidinecarboxylate | |
| ZiMo12.376 | N-{[2-(4-acetyl-1-piperazinyl)-3- | 13.43 |
| | pyridinyl]methyl}-3-(trifluoromethyl)- | |
| | 2-pyridinamine | |
| ZiMo12.533 | N-[(6-{[(2R*,6S*)-2,6-dimethyl-4- | 13.42 |
| | morpholinyl]carbonyl}imidazo[2,1- | |
| | b][1,3]thiazol-5-yl)methyl]-2-[(1-methyl- | |
| | 1H-tetrazol-5-yl)thio]ethanamine | |
| ZiMo12.418 | 4-{[1-(3-methoxypropanoyl)-4- | 13.25 |
| | piperidinyl]oxy}-N-[2-(3-methyl-2- | |
| | pyridinyl)ethyl]benzamide | |
| ZiMo12.139 | ethyl 1-[(5-methyl-1H-indazol-3- | 13.21 |
| | yl)carbonyl]-4-(tetrahydro-2H-pyran- | |
| | 2-ylmethyl)-4-piperidinecarboxylate | |
| ZiMo12.370 | 2-[4-(3,4-difluorobenzyl)-1-(4-methoxy-2,3- | 13.11 |
| | dimethylbenzyl)-2-piperazinyl]ethanol | |
| ZiMo12.566 | methyl 2-{[{{1-[2-(3-fluorophenyl)ethyl]-4- | 13.05 |
| | piperidinyl}methyl)(methyl) | |
| | amino]carbonyl}benzoate | |
| ZiMo12.480 | 1′-(3-methylphenyl)-3-[3-(4-morpholinyl)- | 12.95 |
| | 3-oxopropyl]-1,4′-bipiperidine | |
| ZiMo12.155 | N-(2,4-dimethoxybenzyl)-3-[1-(3- | 12.94 |
| | fluorobenzyl)-3-piperidinyl]propanamide | |
| ZiMo12.458 | 1-benzyl-4-({5-[(2-methoxy- | 12.76 |
| | 4-methylphenoxy)methyl]-3- | |
| | isoxazolyl}carbonyl)piperazine | |
| ZiMo12.468 | N-butyl-4-{[1-(cyclopropylcarbonyl)- | 12.72 |
| | 4-piperidinyl]oxy}benzamide | |
| ZiMo12.540 | 3-chloro-4-{[1-(methylsulfonyl)- | 12.45 |
| | 4-piperidinyl]oxy}-N-[(2-methyl-1,3- | |
| | thiazol-4-yl)methyl]benzamide | |
| ZiMo12.191 | 1-[(2E)-4-methyl-2-penten-1-yl]-4-[2-(1- | 12.35 |
| | pyrrolidinylcarbonyl)phenoxy]piperidine | |
| ZiMo12.308 | 4-(2-oxo-1-pyrrolidinyl)-N-[1-(2- | 12.29 |
| | phenylethyl)-3-piperidinyl]butanamide | |
| ZiMo12.29 | 2-amino-N-{[1-(2,3-dihydro-1H-inden- | 12.26 |
| | 2-yl)-4-piperidinyl]methyl}-6- | |
| | methyl-N-(tetrahydro-2-furanylmethyl)- | |
| | 4-pyrimidinecarboxamide | |
| ZiMo12.59 | methyl 6-({[1-(2-fluorophenyl)- | 12.23 |
| | 4,5,6,7-tetrahydro-1H-indazol-4- | |
| | yl]amino}carbonyl)nicotinate | |
| ZiMo12.398 | N-({1-[2-(4-chlorophenyl)ethyl]-4- | 12.15 |
| | piperidinyl}methyl)-N-methyl-6- | |
| | quinoxalinecarboxamide | |
| ZiMo12.426 | methyl 5-(3-{3-[(4-methoxy-2- | 12.08 |
| | methylphenyl)amino]-3-oxopropyl}-1- | |
| | piperidinyl)-5-oxopentanoate | |
| ZiMo12.98 | 2-[1-(2,4-dimethoxybenzyl)-3-oxo-2- | 11.8 |
| | piperazinyl]-N-[2-(3- | |
| | pyridinyl)ethyl]acetamide | |
| ZiMo12.58 | N-(3,4-dimethoxyphenyl)-1-(3- | 11.79 |
| | methoxybenzoyl)-3-piperidinamine | |
| ZiMo12.279 | N-{[3-(2,3-dihydro-1,4-benzodioxin- | 11.72 |
| | 6-yl)-1-methyl-1H-pyrazol-4- | |
| | yl]methyl}-3-(1H-pyrazol-1-yl)- | |
| | 1-propanamine | |
| ZiMo12.192 | 1-{4-[(3-{[2-(2-methoxyethyl)- | 11.64 |
| | 1-piperidinyl]carbonyl}-5- | |
| | isoxazolyl)methoxy]phenyl}ethanone | |
| ZiMo12.229 | 4-{3-[benzyl(methyl)amino]-3- | 11.58 |
| | oxopropyl}-N-[3-(methylthio)phenyl]-1- | |
| | piperidinecarboxamide | |
| ZiMo12.129 | N-ethyl-1-[2-(4-methoxyphenyl)ethyl]- | 11.43 |
| | N-(2-methyl-2-propen-1-yl)-6- | |
| | oxo-3-piperidinecarboxamide | |
| ZiMo12.48 | (3R*,4R*)-1-(2-hydroxy-5- | 11.19 |
| | methoxybenzyl)-4-[4-(2-pyridinyl)-1- | |
| | piperazinyl]-3-piperidinol | |
| ZiMo12.302 | N-[2-(4-methyl-5,6,7,8-tetrahydro- | 11.17 |
| | 2-quinazolinyl)ethyl]-6-oxo-1-(2- | |
| | pyridinylmethyl)-3-piperidinecarboxamide | |
| ZiMo12.323 | ethyl 1-[(1-ethyl-5-methyl- | 11.15 |
| | 1H-pyrazol-4-yl)methyl]-3-(3- | |
| | methoxybenzyl)-3-piperidinecarboxylate | |
| ZiMo12.281 | N-(2,4-dimethoxybenzyl)-3-[1-(2- | 10.94 |
| | thienylmethyl)-3-piperidinyl]propanamide | |
| ZiMo12.405 | ethyl 1-[(4-chlorophenoxy)acetyl]- | 10.79 |
| | 4-(tetrahydro-2H-pyran-2- | |
| | ylmethyl)-4-piperidinecarboxylate | |
| ZiMo12.104 | ethyl 1-[(2-methyl-3-pyridinyl)carbonyl]- | 10.77 |
| | 4-(3-phenylpropyl)-4- | |
| | piperidinecarboxylate | |
| ZiMo12.173 | N-{1-[1-(4-fluoro-2-methoxybenzyl)- | 10.75 |
| | 4-piperidinyl]-1H-pyrazol-5- | |
| | yl}tetrahydro-3-furancarboxamide | |
| ZiMo12.194 | 1-[2-({[3-(4-cyclohexylphenyl)- | 10.73 |
| | 1-methyl-1H-pyrazol-4- | |
| | yl]methyl}amino)ethyl]-4-piperidinol | |
| ZiMo12.109 | 3-fluoro-N-{1-[1-(2-methyl-3-furoyl)- | 10.61 |
| | 4-piperidinyl]-1H-pyrazol-5- | |
| | yl}benzamide | |
| ZiMo12.88 | N-({1-[2-(2-methoxyphenyl)ethyl]- | 10.55 |
| | 4-piperidinyl}methyl)-N-methyl-2- | |
| | (2-methylphenyl)acetamide | |
| ZiMo12.601 | (3aS*,6aR*)-5-isopropyl-3-[3-(4- | 10.54 |
| | methoxyphenyl)propyl]hexahydro- | |
| | 2H-pyrrolo[3,4-d][1,3]oxazol-2-one | |
| ZiMo12.86 | 1-{[1-(2-fluorobenzyl)-2-(methylsulfonyl)- | 10.5 |
| | 1H-imidazol-5-yl]methyl}-4- | |
| | (3-methoxypropyl)piperidine | |
| ZiMo12.213 | ethyl 3-(2-methylbenzyl)-1-[(2-methyl- | 10.34 |
| | 1,3-thiazol-5-yl)methyl]-3- | |
| | piperidinecarboxylate | |
| ZiMo12.185 | 2-(ethyl{[3-(3-fluoro-4-methoxyphenyl)- | 10.3 |
| | 1-(2-methylphenyl)-1H- | |
| | pyrazol-4-yl]methyl}amino)ethanol | |
| ZiMo12.576 | 2-({[1-(3,5-difluorophenyl)- | 10.19 |
| | 4,5,6,7-tetrahydro-1H-indazol-4- | |
| | yl]amino}methyl)-6-ethoxyphenol | |
| ZiMo12.534 | methyl 5-[7-(2,3-difluorobenzyl)- | 10.1 |
| | 2,7-diazaspiro[4.5]dec-2-yl]-5- | |
| | oxopentanoate | |
| ZiMo12.602 | 1-(cyclopropylmethyl)-N-[4-(3- | 10.1 |
| | pyridinyloxy)phenyl]-4- | |
| | piperidinecarboxamide | |
| ZiMo12.115 | (1-{[2-(3-phenylpropyl)-1,3- | 9.8 |
| | benzoxazol-5-yl]carbonyl}-2- | |
| | piperidinyl)methanol | |
| ZiMo12.40 | 1-ethyl-4-(2-{[1-(2-pyridinylmethyl)-4- | 9.74 |
| | piperidinyl]oxy}benzoyl)piperazine | |
| ZiMo12.261 | methyl N-[(1-cycloheptyl-6- | 9.67 |
| | oxo-3-piperidinyl)carbonyl]-N- | |
| | methylglycinate | |
| ZiMo12.360 | ethyl 4-(4-methoxybenzyl)-1-[2- | 9.56 |
| | (methylthio)benzoyl]-4- | |
| | piperidinecarboxylate | |
| ZiMo12.53 | 4-methoxy-N-(2-methoxyethyl)- | 9.52 |
| | 2-({1-[(1-methyl-1H-imidazol-2- | |
| | yl)methyl]-4-piperidinyl}oxy)benzamide | |
| ZiMo12.116 | N-(3-hydroxy-2,2- | 9.44 |
| | dimethylpropyl)-5-[(5,6,7,8- | |
| | tetrahydro-2-naphthalenyloxy)methyl]- | |
| | 3-isoxazolecarboxamide | |
| ZiMo12.358 | (2-fluoro-5-methoxybenzyl)(8-methoxy- | 9.26 |
| | 3,4-dihydro-2H-chromen-3-yl)amine | |
| ZiMo12.430 | 5-{[(6-methyl-3-pyridinyl)oxy]methyl}- | 9.22 |
| | N-[2-(2-oxo-1-piperidinyl)ethyl]- | |
| | 3-isoxazolecarboxamide | |
| ZiMo12.216 | N-[2-(methylthio)ethyl]-6-oxo-1- | 9.19 |
| | [3-(trifluoromethyl)benzyl]-3- | |
| | piperidinecarboxamide | |
| ZiMo12.117 | N-{[1-(2,3-dihydro-1H-inden-2-yl)- | 9.12 |
| | 4-piperidinyl]methyl}-4-methyl-N- | |
| | (tetrahydro-2-furanylmethyl)-1,3- | |
| | thiazole-5-carboxamide | |
| ZiMo12.156 | 4-({1-butyl-2-[(4- | 8.89 |
| | methylpentyl)sulfonyl]-1H-imidazol-5- | |
| | yl}methyl)thiomorpholine | |
| ZiMo12.447 | 1-(1-cyclopentyl-4-piperidinyl)-N- | 8.84 |
| | (3-methoxybenzyl)-N-(tetrahydro-2- | |
| | furanylmethyl)methanamine | |
| ZiMo12.592 | {1-[3-(2-methoxyphenyl)propanoyl]-3- | 8.51 |
| | piperidinyl}[3- | |
| | (trifluoromethyl)phenyl]methanone | |
| ZiMo12.96 | 4-{[3-(3-fluoro-4-methoxyphenyl)- | 8.48 |
| | 1-(2-methylphenyl)-1H-pyrazol-4- | |
| | yl]methyl}morpholine | |
| ZiMo12.257 | 4-{[1-(2-chloro-4-fluorobenzyl)-1H-1,2,3- | 8.37 |
| | triazol-4-yl]carbonyl}morpholine | |
| ZiMo12.434 | 3-[4-(2-methoxyphenyl)-1-piperazinyl]- | 8.08 |
| | 1′-methyl-1,4′-bipiperidine | |
| ZiMo12.296 | N-{[1-(2-fluorobenzyl)- | 8.04 |
| | 4-piperidinyl]methyl}- | |
| | 4-methoxy-N-(2-methoxyethyl)- | |
| | 3-methylbenzamide | |
| ZiMo12.572 | N-(tert-butyl)-1′-(2-phenylethyl)- | 7.98 |
| | 1,4′-bipiperidine-3-carboxamide | |
| ZiMo12.515 | 3-chloro-4-{[1-(methylsulfonyl)- | 7.94 |
| | 4-piperidinyl]oxy}- | |
| | N-[3-(methylthio)propyl]benzamide | |
| ZiMo12.553 | N-cyclopropyl-3-[1-(2,4-dimethoxy-3- | 7.76 |
| | methylbenzyl)-3-piperidinyl]propanamide | |
| ZiMo12.235 | 5-[(4-acetyl-2-methoxyphenoxy)methyl]- | 7.72 |
| | N-methyl-N-(1,3-thiazol-2- | |
| | ylmethyl)-3-isoxazolecarboxamide | |
| ZiMo12.255 | N-[(1-ethyl-1H-pyrazol-4-yl)methyl]- | 7.7 |
| | 3-[(1-isopropyl-4-piperidinyl)oxy]- | |
| | 4-methoxybenzamide | |
| ZiMo12.84 | 4-[5-({[1-(3-methoxybenzyl)-3- | 7.29 |
| | piperidinyl]amino}methyl)-2-thienyl]-2- | |
| | methyl-3-butyn-2-ol | |
| ZiMo12.249 | ethyl 3-benzyl-1-[(1-ethyl-5-methyl-1H- | 7.27 |
| | pyrazol-4-yl)methyl]-3-piperidinecarboxylate | |
| ZiMo12.2 | 2-(4-ethyl-1-piperazinyl)-N-[2-(4- | 7.25 |
| | methoxyphenyl)ethyl]-N-[(1-methyl- | |
| | 4-piperidinyl)methyl]acetamide | |
| ZiMo12.497 | N-methyl-N-(2-phenylethyl)-1′-(2- | 7.23 |
| | pyridinylmethyl)-1,4′-bipiperidin-3- | |
| | amine | |
| ZiMo12.396 | ethyl 1-acetyl-3-(4-fluorobenzyl)-3- | 6.92 |
| | piperidinecarboxylate | |
| ZiMo12.509 | N-[1-(2,3-dimethylphenyl)-4,5,6,7- | 6.91 |
| | tetrahydro-1H-indazol-4-yl]-4-(2- | |
| | oxo-1-piperidinyl)butanamide | |
| ZiMo12.505 | ethyl 4-{[(1-cycloheptyl-6-oxo- | 6.85 |
| | 3-piperidinyl)carbonyl]amino}-1- | |
| | piperidinecarboxylate | |
| ZiMo12.54 | N-({1-[(3-methyl-2-thienyl) | 6.84 |
| | sulfonyl]-3-piperidinyl}methyl)-1- | |
| | phenylmethanesulfonamide | |
| ZiMo12.170 | N-benzyl-N-methyl-3-({[2-(4- | 6.77 |
| | pyridinyl)ethyl]amino}methyl)imidazo[1,2- | |
| | a]pyridine-2-carboxamide | |
| ZiMo12.440 | N-(2-chlorobenzyl)-3-[1- | 6.76 |
| | (4-methoxybenzoyl)-3- | |
| | piperidinyl]propanamide | |
| ZiMo12.186 | (3R*,4R*)-1-(4-fluoro-3-methoxybenzyl)- | 6.72 |
| | 4-(4-methyl-1-piperazinyl)-3- | |
| | piperidinol | |
| ZiMo12.65 | 5-[(4-acetylphenoxy)methyl]-N-(2- | 6.44 |
| | phenylpropyl)-3-isoxazolecarboxamide | |
| ZiMo12.424 | 1-{[3-(2-fluoro-4-methoxyphenyl)- | 6.4 |
| | 1-phenyl-1H-pyrazol-4-yl]methyl}- | |
| | 4-methoxypiperidine | |
| ZiMo12.278 | N-allyl-6-[3-(trifluoro- | 6.1 |
| | methyl)phenyl]imidazo[2,1- | |
| | b][1,3]thiazole-3-carboxamide | |
| ZiMo12.408 | N-cyclopropyl-2-[1-(4-methoxy-3- | 6.09 |
| | methylbenzyl)-3-oxo-2- | |
| | piperazinyl]acetamide | |
| ZiMo12.437 | N-butyl-3-({[3-(trifluoro- | 6.09 |
| | methyl)benzoyl]amino}methyl)-1- | |
| | piperidinecarboxamide | |
| ZiMo12.89 | N-{[1-(4,5,6,7-tetrahydro-2- | 6.08 |
| | benzothien-1-ylcarbonyl)-3- | |
| | piperidinyl]methyl}-2-thiophenesulfonamide | |
| ZiMo12.590 | methyl N-{2-[(1-acetyl-4-piperidinyl)oxy]- | 5.9 |
| | 5-methoxybenzoyl}-L-valinate | |
| ZiMo12.306 | 2-methoxy-N-(1-{1-[(5-methyl-2- | 5.86 |
| | thienyl)methyl]-4-piperidinyl}-1H- | |
| | pyrazol-5-yl)-2-phenylacetamide | |
| ZiMo12.113 | ethyl 3-[(2E)-3-phenyl-2-propen- | 5.81 |
| | 1-yl]-1-(3-pyridinylacetyl)-3- | |
| | piperidinecarboxylate | |
| ZiMo12.355 | N-(tert-butyl)-5-chloro-2-({1-[(4- | 5.8 |
| | methyl-1H-imidazol-2-yl)methyl]-4- | |
| | piperidinyl}oxy)benzamide | |
| ZiMo12.529 | N-ethyl-4,4,4-trifluoro-N-({1-[2- | 5.75 |
| | (2-methylphenyl)ethyl]-4- | |
| | piperidinyl}methyl)butanamide | |
| ZiMo12.176 | N-(1-cycloheptyl-3-piperidinyl)- | 5.61 |
| | N′-1H-indol-3-ylurea | |
| ZiMo12.119 | ethyl 1-(3,3-dimethylbutanoyl)-3-(4- | 5.56 |
| | fluorobenzyl)-3-piperidinecarboxylate | |
| ZiMo12.385 | 2-({1-[(4-isopropyl-1,3-thiazol-2- | 5.43 |
| | yl)methyl]-4-piperidinyl}oxy)-4- | |
| | methoxy-N-(2-methoxyethyl)benzamide | |
| ZiMo12.544 | N-(2,3-dihydro-1,4-benzodioxin- | 5.41 |
| | 6-yl)-3-[1-(4-pyridinylmethyl)-4- | |
| | piperidinyl]propanamide | |
| ZiMo12.284 | 3-{1-[(5-chloro-2-thienyl)methyl]- | 5.19 |
| | 4-piperidinyl}-N-[(1-ethyl-2- | |
| | pyrrolidinyl)methyl]propanamide | |
| ZiMo12.564 | 3-{[1-(4-fluorophenyl)-6,6-dimethyl- | 5.01 |
| | 4,5,6,7-tetrahydro-1H-indazol-4- | |
| | yllamino}-2,2-dimethyl-1-propanol | |
| ZiMo12.137 | 2-[1-(2-fluorobenzyl)-4-(2,3,6- | 4.85 |
| | trifluorobenzyl)-2-piperazinyl]ethanol | |
| ZiMo12.329 | N-(3,5-dimethoxyphenyl)-3-[1-(4- | 4.67 |
| | fluoro-3-methoxybenzyl)-3- | |
| | piperidinyl]propanamide | |
| ZiMo12.379 | 1-(1-cyclopentyl-4-piperidinyl)-N- | 4.6 |
| | (3-fluorobenzyl)-N-(tetrahydro-2- | |
| | furanylmethyl)methanamine | |
| ZiMo12.135 | 3-(1-{[2-(dimethylamino)-1,3-thiazol- | 4.58 |
| | 5-yl]methyl}-4-piperidinyl)-N-(3- | |
| | pyridinylmethyl)propanamide | |
| ZiMo12.91 | N-(3-pyridinylmethyl)-1′-(1,2,3,4- | 4.48 |
| | tetrahydro-2-naphthalenyl)-1,4′- | |
| | bipiperidine-4-carboxamide | |
| ZiMo12.266 | N-{[1-(2-methoxybenzyl)-4- | 3.97 |
| | piperidinyl]methyl}-N-(2-methoxyethyl)-3- | |
| | (methylthio)propanamide | |
| ZiMo12.226 | (3-ethoxy-4-methoxybenzyl){1-[1- | 3.94 |
| | (3-fluorophenyl)-5-methyl-1H- | |
| | pyrazol-4-yl]ethyl}amine | |
| ZiMo12.456 | N-(3,5-dimethoxybenzyl)-2-[1-(2- | 3.93 |
| | ethoxybenzyl)-3-oxo-2-piperazinyl]acetamide | |
| ZiMo12.366 | N-(1-cycloheptyl-3-piperidinyl)- | 3.89 |
| | 2-methyl-3-furamide | |
| ZiMo12.420 | N-{[1-(1,3-benzothiazol-2-ylmethyl)-3- | 3.86 |
| | piperidinyl]methyl}-1- | |
| | phenylmethanesulfonamide | |
| ZiMo12.183 | ethyl 1-(1,3-benzodioxol-4-ylmethyl)-3-(3- | 3.83 |
| | methoxybenzyl)-3-piperidinecarboxylate | |
| ZiMo12.496 | 2-{4-[6-(1-azepanyl)[1,2,5]oxadiazolo[3,4- | 3.83 |
| | b]pyrazin-5-yl]-1-piperazinyl}ethanol | |
| ZiMo12.331 | 1-(3-ethoxybenzyl)-4-[2-(1-piperi- | 3.81 |
| | dinylcarbonyl)phenoxy]piperidine | |
| ZiMo12.486 | 3-({3-[(4-fluorophenyl)amino]-1- | 3.7 |
| | piperidinyl}carbonyl)-2H-chromen-2- | |
| | one | |
| ZiMo12.256 | N-isopropyl-1′-{[5-methyl-2-(2-thienyl)- | 3.69 |
| | 1,3-oxazol-4-yl]methyl}-1,4′- | |
| | bipiperidine-4-carboxamide | |
| ZiMo12.436 | N-{[2-(4-acetyl-1-piperazinyl)-3- | 3.5 |
| | pyridinyl]methyl}-2,6-dimethoxy-4- | |
| | pyrimidinamine | |
| ZiMo12.18 | N-(1-cycloheptyl-3-piperidinyl)- | 3.42 |
| | 3-(2-fluorophenyl)propanamide | |
| ZiMo12.125 | 1-{4-[(cycloheptylamino)methyl]- | 3.3 |
| | 2-methoxyphenoxy}-3-(4- | |
| | thiomorpholinyl)-2-propanol | |
| ZiMo12.209 | 1-[1-(3-fluorophenyl)-5-methyl- | 3.3 |
| | 1H-pyrazol-4-yl]-N-(2,3,4- | |
| | trimethoxybenzyl)ethanamine | |
| ZiMo12.562 | 2-(methylthio)-N-[1-(2-phenylethyl)- | 3.22 |
| | 3-piperidinyl]benzamide | |
| ZiMo12.527 | 1-(1-azocanyl)-3-(5-{[4-(hydroxymethyl)- | 3.15 |
| | 1-piperidinyl]methyl}-2- | |
| | methoxyphenoxy)-2-propanol | |
| ZiMo12.557 | N-[(1-hydroxycyclohexyl)methyl]- | 3.11 |
| | 6-phenylimidazo[2,1-b][1,3]thiazole- | |
| | 3-carboxamide | |
| ZiMo12.168 | 1-[2-(benzylsulfonyl)-1-isobutyl-1H- | 3.05 |
| | imidazol-5-yl]-N-methyl-N-[(5- | |
| | methyl-2-furyl)methyl]methanamine | |
| ZiMo12.386 | N-(3-isoxazolylmethyl)-6- | 2.92 |
| | phenylimidazo[2,1-b][1,3]thiazole-3- | |
| | carboxamide | |
| ZiMo12.361 | N-(1-{1-[3-(methylthio)propanoyl]- | 2.85 |
| | 4-piperidinyl}-1H-pyrazol-5-yl)-3- | |
| | phenylpropanamide | |
| ZiMo12.599 | (2,1,3-benzothiadiazol-5- | 2.8 |
| | ylmethyl){{1-[2-(4-fluorophenyl)ethyl]-4- | |
| | piperidinyl}methyl)methylamine | |
| ZiMo12.343 | (1R)-1-(3-methoxyphenyl)-N-{[8-methyl- | 2.79 |
| | 2-(4-morpholinylcarbonyl)imidazo[1,2- | |
| | a]pyridin-3-yl]methyl}ethanamine | |
| ZiMo12.605 | 1-[3-(2,4-difluorophenyl)-1H-pyrazol- | 2.74 |
| | 4-yl]-N-(2,3-dihydro-1-benzofuran-2- | |
| | ylmethyl)-N-methylmethanamine | |
| ZiMo12.402 | N-{[1-(1,3-thiazol-5-ylcarbonyl)-3- | 2.72 |
| | piperidinyl]methyl}-4-biphenylcarboxamide | |
| ZiMo12.593 | ethyl 4-(3-phenylpropyl)-1- | 2.7 |
| | ([1,2,4]triazolo[1,5-a]pyrimidin-6-ylacetyl)- | |
| | 4-piperidinecarboxylate | |
| ZiMo12.552 | 3-(3-methoxyphenyl)-N-({1-[2- | 2.62 |
| | (2-methoxyphenyl)ethyl]-4- | |
| | piperidinyl}methyl)-N-methylpropanamide | |
| ZiMo12.407 | 4,4,4-trifluoro-N-{[1-(2-fluorobenzyl)- | 2.49 |
| | 4-piperidinyl]methyl}-N- | |
| | (tetrahydro-2-furanylmethyl)butanamide | |
| ZiMo12.108 | 2-{[1-(2-fluorobenzyl)-1H-1,2,3-triazol- | 2.38 |
| | 4-yl]carbonyl}-6,7-dimethoxy- | |
| | 1,2,3,4-tetrahydroisoquinoline | |
| ZiMo12.442 | N-(2,4-dimethoxybenzyl)-3-[1-(3- | 2.38 |
| | methoxybenzyl)-3-piperidinyl]propanamide | |
| ZiMo12.187 | methyl (2S*,4S*,5R*)-5-(2,3- | 2.04 |
| | difluorophenyl)-4-({[(1,5-dimethyl-1H- | |
| | pyrazol-4-yl)methyl]amino}carbonyl)-1- | |
| | methyl-2-pyrrolidinecarboxylate | |
| ZiMo12.51 | 2-[4-(4-ethylbenzyl)-1-(2-fluorobenzyl)- | 2.01 |
| | 2-piperazinyl]ethanol | |
| ZiMo12.286 | 1-[(2E)-3-(3,4-dimethoxyphenyl)- | 1.95 |
| | 2-propenoyl]-N-(3-fluorophenyl)-3- | |
| | piperidinamine | |
| ZiMo12.289 | 3-(1-{[5-(methoxymethyl)-2- | 1.8 |
| | furyl]methyl}-3-piperidinyl)-N-(4- | |
| | methoxy-2-methylphenyl)propanamide | |
| ZiMo12.462 | 3-[1-(2,5-difluorobenzyl)-3-piperidinyl]- | 1.7 |
| | N-(2,4-dimethoxybenzyl)propanamide | |
| ZiMo12.87 | 3-{2-[4-(cyclopropylmethyl)-1- | 1.61 |
| | piperazinyl]-2-oxoethyl}-4-(3- | |
| | fluorobenzyl)-2-piperazinone | |
| ZiMo12.277 | N-[2-(3,5-dimethyl-1H-pyrazol-1- | 1.42 |
| | yl)ethyl]-2-[1-(1-naphthylmethyl)-3- | |
| | oxo-2-piperazinyl]acetamide | |
| ZiMo12.252 | 5-(2,3-dihydro-1,4-benzoxazepin- | 1.15 |
| | 4(5H)-ylcarbonyl)-1-[2-(4- | |
| | morpholinyl)ethyl]-2-piperidinone | |
| ZiMo12.474 | N-(3′-methyl-4-biphenylyl)-1-(2- | 1.08 |
| | pyrazinylcarbonyl)-4-piperidinecarboxamide | |
| ZiMo12.83 | 1′-ethyl-N-(3-pyridinylmethyl)- | 1.03 |
| | 1,4′-bipiperidine-4-carboxamide | |
| ZiMo12.547 | 2-(3,4-dimethoxybenzyl)-N-[2- | 0.84 |
| | (methylthio)ethyl]-1,3-benzoxazole-6- | |
| | carboxamide | |
| ZiMo12.580 | 1-(6-chloro-1,3-benzodioxol-5-yl)-N-[(1- | 0.8 |
| | cyclopentyl-4-piperidinyl) methyl]-N- | |
| | (tetrahydro-2-furanylmethyl)methanamine | |
| ZiMo12.149 | 1-(2-fluorobenzyl)-N-[2-(3- | 0.74 |
| | pyridinyloxy)propyl]-1H-1,2,3-triazole-4- | |
| | carboxamide | |
| ZiMo12.412 | 1-cyclohexyl-4-{[1-(3,4-difluorobenzyl)- | 0.69 |
| | 3-oxo-2-piperazinyl]acetyl}-2- | |
| | piperazinone | |
| ZiMo12.9 | N-{1-[5-methyl-1-(2-methylphenyl)-1H- | 0.66 |
| | pyrazol-4-ylethyl}cyclopropanecarboxamide | |
| ZiMo12.132 | 2-[4-[(1-ethyl-3,5-dimethyl-1H-pyrazol- | 0.65 |
| | 4-yl)methyl]-1-(2-fluoro-4- | |
| | methoxybenzyl)-2-piperazinyl]ethanol | |
| ZiMo12.123 | 3-(2-methoxyphenyl)-N-[2-(3- | 0.62 |
| | pyridinylmethyl)-1,2,3,4-tetrahydro-7- | |
| | isoquinolinyl]propanamide | |
| ZiMo12.372 | 1-[5-(1-azepanylmethyl)-2-methoxyphenoxy]- | 0.55 |
| | 3-(4-methyl-1-piperazinyl)-2-propanol | |
| ZiMo12.72 | 5-[(4-chloro-3,5- | 0.42 |
| | dimethylphenoxy)methyl]-N-methyl-N-(2- | |
| | pyrazinylmethyl)-3-isoxazolecarboxamide | |
| ZiMo12.64 | 1-(4-fluorobenzyl)-4-{[2-(3- | 0.41 |
| | methoxyphenyl)-1-pyrrolidinyl]carbonyl}- | |
| | 1H-1,2,3-triazole | |
| ZiMo12.364 | N-methyl-N-[(5-methyl-2-furyl)methyl]- | 0.32 |
| | 2-{[1-(2-pyridinylmethyl)-4- | |
| | piperidinyl]oxy}benzamide | |
| ZiMo12.227 | N-(2,4-dimethoxybenzyl)-3-[1- | 0.26 |
| | (2-hydroxy-5-methoxybenzyl)-3- | |
| | piperidinyl]propanamide | |
| ZiMo12.381 | 2-cyclopropyl-6-[(4-phenyl-3,6- | 0.26 |
| | dihydro-1(2H)-pyridinyl)carbonyl]-1,3- | |
| | benzoxazole | |
| ZiMo12.543 | 2-[4-[(1,5-dimethyl-1H-pyrazol- | 0.21 |
| | 4-yl)methyl]-1-(4-methoxy-3- | |
| | methylbenzyl)-2-piperazinyl]ethanol | |
| ZiMo12.417 | N-isopropyl-1′-[3-(5-methyl-2- | 0.1 |
| | furyl)benzyl]-1,4′-bipiperidine-4- | |
| | carboxamide | |
| ZiMo12.188 | ethyl 4-(4-chlorobenzyl)-1-(3-hydroxy-4- | −0.06 |
| | methoxybenzyl)-4-piperidinecarboxylate | |
| ZiMo12.110 | 1″-propyl-N-(3-pyridinylmethyl)-1,4′:1′, | −0.12 |
| | 4″-terpiperidine-4-carboxamide | |
| ZiMo12.525 | N-[3-(4-methyl-1,3-thiazol-5- | −0.12 |
| | yl)propyl]-1-(tetrahydro-2- | |
| | furanylmethyl)-1H-1,2,3-triazole- | |
| | 4-carboxamide | |
| ZiMo12.233 | 2-{1-(3,5-dimethoxybenzyl)-4-[(1- | −0.18 |
| | isopropyl-1H-pyrazol-4-yl)methyl]- | |
| | 2-piperazinyl}ethanol | |
| ZiMo12.419 | N-[4-({methyl[1-(2-phenylethyl)-3- | −0.22 |
| | piperidinyl]amino}methyl)phenyl]acetamide | |
| ZiMo12.573 | N-methyl-N-[(3-methyl-4- | −0.25 |
| | pyridinyl)methyl]-5-{[4-(1H-1,2,4-triazol-1- | |
| | yl)phenoxy]methyl}-3-isoxazolecarboxamide | |
| ZiMo12.254 | N-[(5-methyl-2-furyl)methyl]-5- | −0.48 |
| | {[4-(1H-pyrazol-1-ylmethyl)-1- | |
| | piperidinyl]carbonyl}-2-pyridinamine | |
| ZiMo12.93 | 2-(dimethylamino)-N-[1-(2-phenylethyl)- | −0.51 |
| | 3-piperidinyl]benzamide | |
| ZiMo12.136 | ethyl 1-{[6-(2-fluorophenyl)imidazo[2,1- | −0.51 |
| | b][1,3]thiazol-3-yl]carbonyl}- | |
| | 4-piperidinecarboxylate | |
| ZiMo12.221 | methyl 3-{[(1-cycloheptyl-3- | −0.51 |
| | piperidinyl)amino]carbonyl}benzoate | |
| ZiMo12.301 | 1-(3-methoxybenzyl)-N-[3-(1H- | −0.51 |
| | pyrazol-1-yl)benzyl]-3-piperidinamine | |
| ZiMo12.13 | N-{1-[1-(3-phenoxypropanoyl)- | −0.53 |
| | 4-piperidinyl]-1H-pyrazol-5- | |
| | yl}tetrahydro-3-furancarboxamide | |
| ZiMo12.217 | N~1~-[1-(3,5-dimethylphenyl)- | −0.68 |
| | 4,5,6,7-tetrahydro-1H-indazol-4-yl]- | |
| | 1,1-cyclopropanedicarboxamide | |
| ZiMo12.283 | N-[(2-phenoxy-3-pyridinyl)methyl]- | −0.68 |
| | 1,3-benzodioxole-5-carboxamide | |
| ZiMo12.208 | 5-[(3-acetylphenoxy)methyl]-N-isopropyl- | −1.2 |
| | N-[(1-methyl-1H-pyrazol-4- | |
| | yl)methyl]-3-isoxazolecarboxamide | |
| ZiMo12.203 | 2-[1-(4-methoxy-3-methylbenzyl)-4-(3- | −1.21 |
| | methylbenzyl)-2-piperazinyl]ethanol | |
| ZiMo12.128 | 4-{2-[4-({4-[(2-methylphenyl)thio]-1- | −1.22 |
| | piperidinyl}carbonyl)-1H-1,2,3- | |
| | triazol-1-yllethyl}morpholine | |
| ZiMo12.202 | N-allyl-1′-(4-pyridinylmethyl)-1,4′- | −1.26 |
| | bipiperidine-3-carboxamide | |
| ZiMo12.550 | 2-[(1-cyclopentyl-4-piperidinyl)oxy]-N- | −1.28 |
| | (2-hydroxypropyl)-5-methoxybenzamide | |
| ZiMo12.528 | N-methyl-1-(7-methyl-1H-benzimidazol- | −1.31 |
| | 2-yl)-N-{[2-(methylsulfonyl)- | |
| | 1-(tetrahydro-2-furanylmethyl)-1H-imidazol- | |
| | 5-yl]methyl}methanamine | |
| ZiMo12.124 | 4-{[3-(2-hydroxyethyl)-4-(3- | −1.47 |
| | methoxybenzyl)-1-piperazinyl]methyl}-2- | |
| | methoxyphenol | |
| ZiMo12.453 | N-{2-[(2-amino-5-pyrimidinyl)methyl]- | −1.62 |
| | 1,2,3,4-tetrahydro-7-isoquinolinyl}-4- | |
| | (trifluoromethyl)benzamide | |
| ZiMo12.549 | 1-(2-methylphenyl)-4-[1- | −1.84 |
| | (3-phenoxypropanoyl)- | |
| | 3-piperidinyl]piperazine | |
| ZiMo12.318 | 5-({4-[2-(1H-pyrazol-1-yl)ethyl]- | −1.88 |
| | 1-piperidinyl}carbonyl)-1-(2- | |
| | pyridinylmethyl)-2-piperidinone | |
| ZiMo12.264 | (3S*)-1-isobutyl-3-methyl-4-{4-[5- | −1.97 |
| | (4-morpholinylmethyl)-1H-tetrazol- | |
| | 1-yl]butanoyl}-2-piperazinone | |
| ZiMo12.243 | methyl 4-({4-[3-(1- | −2.02 |
| | pyrrolidinylcarbonyl)phenoxy]-1- | |
| | piperidinyl}methyl)benzoate | |
| ZiMo12.178 | 1-{2-[1-(4-methoxy-3-methylphenyl)- | −2.16 |
| | 1,3,4,9-tetrahydro-2H-beta- | |
| | carbolin-2-yl]-2-oxoethyl}-1,2-dihydro- | |
| | 3,6-pyridazinedione | |
| ZiMo12.368 | 1-(3-furylmethyl)-N-[4-(1H-indol-2- | −2.25 |
| | yl)phenyl]-4-piperidinecarboxamide | |
| ZiMo12.23 | N-(2-methoxyethyl)-3-{[1-(4- | −2.26 |
| | quinolinylmethyl)-4- | |
| | piperidinyl]oxy}benzamide | |
| ZiMo12.506 | ethyl 3-(4-fluorobenzyl)-1-isobutyryl- | −2.38 |
| | 3-piperidinecarboxylate | |
| ZiMo12.416 | 1-[3-(3,4-dimethylphenyl)-1H-pyrazol- | −2.49 |
| | 4-yl]-N-[2-fluoro-5- | |
| | (trifluoromethyl)benzyl]methanamine | |
| ZiMo12.551 | 3-[1-(3-acetylbenzyl)-4-piperidinyl]-N-[(1- | −2.5 |
| | ethyl-2-pyrrolidinyl)methyl]propanamide | |
| ZiMo12.546 | 1′-[(5-methyl-2-thienyl)methyl]-N-(2- | −2.64 |
| | pyridinylmethyl)-1,4′-bipiperidine- | |
| | 4-carboxamide | |
| ZiMo12.449 | 2-(2-fluorobenzyl)-6-{[4- | −2.72 |
| | (2-pyrimidinyl)-1,4-diazepan-1-yl]carbonyl}- | |
| | 1,3-benzoxazole | |
| ZiMo12.469 | 1′-[1-methyl-2-(2-pyridinyl)ethyl]-N-(2- | −2.8 |
| | pyridinylmethyl)-1,4′-bipiperidine- | |
| | 4-carboxamide | |
| ZiMo12.598 | 2-{[6-(2-methylphenyl)imidazo[2,1- | −3.06 |
| | b][1,3]thiazol-3-yl]carbonyl}octa- | |
| | hydropyrrolo[1,2-a]pyrazine | |
| ZiMo12.454 | N-(4-methoxy-2-methylphenyl)-3-[1-(3- | −3.14 |
| | thienylsulfonyl)-3-piperidinyl]propanamide | |
| ZiMo12.413 | 2-[4-(3-fluorobenzyl)-1-(4-methoxy-2,3- | −3.27 |
| | dimethylbenzyl)-2-piperazinyl]ethanol | |
| ZiMo12.293 | 1-(3-methoxybenzyl)-N-{[1-(3-methylphenyl)- | −3.38 |
| | 1H-imidazol-2-yl]methyl}-3-piperidinamine | |
| ZiMo12.271 | 4-{[1-(cyclopropylcarbonyl)- | −3.43 |
| | 4-piperidinyl]oxy}- | |
| | N-(2-hydroxybutyl)benzamide | |
| ZiMo12.76 | 1-(4-fluorobenzyl)-N-[4-(3- | −3.48 |
| | pyridinyloxy)phenyl]-4- | |
| | piperidinecarboxamide | |
| ZiMo12.483 | 2-[4-[(4-isopropyl-1,3-thiazol-2-yl)methyl]- | −3.53 |
| | 1-(4-methoxy-2,3-dimethylbenzyl)- | |
| | 2-piperazinyl]ethanol | |
| ZiMo12.253 | N-isopropyl-2-[2-methyl-4-oxo-1- | −3.54 |
| | (2-phenylethyl)-4,5,6,7-tetrahydro- | |
| | 1H-indol-3-yl]acetamide | |
| ZiMo12.121 | N-(1-{1-[(1-ethyl-1H-pyrazol-4-yl)methyl]- | −3.55 |
| | 4-piperidinyl}-1H-pyrazol-5- | |
| | yl)-2-phenylacetamide | |
| ZiMo12.105 | 6-phenyl-N-(4-pyridinylmethyl)-N- | −3.61 |
| | (tetrahydro-2-furanylmethyl)imidazo[2,1- | |
| | b][1,3]thiazole-3-carboxamide | |
| ZiMo12.524 | 1-[2-(benzylsulfonyl)-1-isobutyl- | −3.62 |
| | 1H-imidazol-5-yl]-N-methyl-N-(4- | |
| | methylbenzyl)methanamine | |
| ZiMo12.400 | 1-[1-(2-methylbenzyl)-4-piperidinyl]- | −3.73 |
| | N-[(1-methyl-1H-imidazol-2- | |
| | yl)methyl]-N-(tetrahydro-2- | |
| | furanylmethyl)methanamine | |
| ZiMo12.120 | 2-(2-fluorobenzyl)-N-methyl- | −3.8 |
| | N-[4-(4-morpholinyl)butyl]-1,3- | |
| | benzoxazole-6-carboxamide | |
| ZiMo12.556 | methyl (2S*,4S*,5R*)-1,2-dimethyl- | −3.81 |
| | 5-(2-methylphenyl)-4-({[2- | |
| | (methylthio)ethyl]amino}carbonyl)- | |
| | 2-pyrrolidinecarboxylate | |
| ZiMo12.30 | 3-{1-[(1-cyclopropyl-5-oxo-3- | −3.83 |
| | pyrrolidinyl)carbonyl]-4-piperidinyl}-N- | |
| | (2,3-dihydro-1,4-benzodioxin-6- | |
| | yl)propanamide | |
| ZiMo12.6 | (3aS*,5S*,9aS*)-2-(2-phenylethyl)- | −3.84 |
| | 5-(8-quinolinyl)hexahydro-7H- | |
| | pyrrolo[3,4-g]pyrrolizin-1(2H)-one | |
| ZiMo12.538 | N-ethyl-1,5-dimethyl-N-({1-[2- | −3.94 |
| | (2-methylphenyl)ethyl]-4- | |
| | piperidinyl}methyl)-1H-pyrazole- | |
| | 3-carboxamide | |
| ZiMo12.415 | 2-{1-[(2E)-3-(2-methoxyphenyl)-2- | −4.04 |
| | propen-1-yl]-3-oxo-2-piperazinyl}- | |
| | N-(3-pyridinylmethyl)acetamide | |
| ZiMo12.31 | N-{[1-(2-fluorobenzyl)-2-(methylsulfonyl)- | −4.11 |
| | 1H-imidazol-5-yl]methyl}-N- | |
| | methyl-2-phenylethanamine | |
| ZiMo12.28 | N-(3-chloro-4-methoxyphenyl)- | −4.14 |
| | 3-{1-[(5-methyl-1H-pyrazol-3- | |
| | yl)carbonyl]-3-piperidinyl}propanamide | |
| ZiMo12.471 | N-(3-methoxypropyl)-6-(4-methyl-3- | −4.16 |
| | phenyl-1-piperazinyl)[1,2,5]oxadiazolo[3,4- | |
| | b]pyrazin-5-amine | |
| ZiMo12.596 | 1-benzoyl-N-(4-fluorophenyl)-3- | −4.51 |
| | piperidinamine | |
| ZiMo12.441 | 2-[4-(2,4-difluorobenzyl)-1-(4-methoxy- | −4.7 |
| | 2,3-dimethylbenzyl)-2-piperazinyl]ethanol | |
| ZiMo12.207 | ethyl 3-(2-fluorobenzyl)-1-[3-(2- | −4.72 |
| | hydroxyethoxy)benzyl]-3- | |
| | piperidinecarboxylate | |
| ZiMo12.201 | 2-(4-{[1-butyl-2-(ethylsulfonyl)-1H-imidazol- | −4.94 |
| | 5-yl]methyl}-1-piperazinyl)ethanol | |
| ZiMo12.579 | 3-({1-[(dimethylamino)sulfonyl]- | −4.95 |
| | 4-piperidinyl}oxy)-N-methyl-N-[(4- | |
| | methyl-1H-imidazol-2-yl)methyl]benzamide | |
| ZiMo12.131 | N-{[2-(2,6-dimethylphenoxy)-3- | −4.96 |
| | pyridinyl]methyl}-2-(2-pyridinyl)acetamide | |
| ZiMo12.577 | 2-{1-(2-ethoxybenzyl)-4-[(5-methyl- | −4.98 |
| | 1-propyl-1H-pyrazol-4-yl)methyl]- | |
| | 2-piperazinyl}ethanol | |
| ZiMo12.212 | 1-(1-cyclopentyl-4-piperidinyl)- | −4.99 |
| | N-(2-fluorobenzyl)-N-(tetrahydro-2- | |
| | furanylmethyl)methanamine | |
| ZiMo12.499 | ethyl 1-{5-[(dimethylamino)methyl]- | −5.02 |
| | 2-furoyl}-3-(2-phenoxyethyl)-3- | |
| | piperidinecarboxylate | |
| ZiMo12.606 | 2-phenyl-4-[4-(tetrahydro-3-thienyl)- | −5.12 |
| | 1-piperazinyl]-6,7-dihydro-5H- | |
| | cyclopenta[d]pyrimidine | |
| ZiMo12.141 | 1-{[2-(benzylsulfonyl)-1-isobutyl- | −5.18 |
| | 1H-imidazol-5-yl]methyl}-2- | |
| | (methoxymethyl)piperidine | |
| ZiMo12.5 | phenyl[1-({3-[4-(trifluoromethyl)phenyl]- | −5.43 |
| | 1,2,4-oxadiazol-5-yl}methyl)- | |
| | 3-piperidinyl]methanone | |
| ZiMo12.450 | N-[2-(3,4-dimethoxyphenyl)ethyl]-N-methyl- | −5.52 |
| | 1-[3-(1H-pyrazol-4-yl)propanoyl]- | |
| | 3-piperidinamine | |
| ZiMo12.508 | 4-(4-{[7-(cyclobutylmethyl)- | −5.54 |
| | 2,7-diazaspiro[4.5]dec-2- | |
| | yl]methyl}phenyl)-3-butyn-1-ol | |
| ZiMo12.451 | N-(3-methoxyphenyl)-3-{1-[3- | −5.59 |
| | (1H-pyrazol-4-yl)propanoyl]-4- | |
| | piperidinyl}propanamide | |
| ZiMo12.395 | (3R*,4R*)-4-(1-azepanyl)-1-[4-(1H- | −5.66 |
| | pyrazol-1-ylmethyl)benzyl]-3- | |
| | piperidinol | |
| ZiMo12.305 | 2-[1-(2-fluorobenzyl)-4-(2-fluoro-4- | −5.68 |
| | methoxybenzyl)-2-piperazinyl]ethanol | |
| ZiMo12.356 | N-[3-(1H-indol-2-yl)phenyl]-1- | −5.77 |
| | propionyl-4-piperidinecarboxamide | |
| ZiMo12.397 | N-{[1-(2-methoxybenzyl)-4- | −5.86 |
| | piperidinyl]methyl}-N-(2-methoxyethyl)-2- | |
| | methyl-1-propanamine | |
| ZiMo12.600 | 1-{[3-(diphenylmethyl)-1,2,4-oxadiazol- | −5.94 |
| | 5-yl]methyl}-2-ethylpiperidine | |
| ZiMo12.295 | N-methyl-5-({[(5-methyl-2- | −5.95 |
| | furyl)methyl]amino}methyl)-N-(2- | |
| | phenylethyl)imidazo[2,1-b][1,3]thiazole- | |
| | 6-carboxamide | |
| ZiMo12.310 | 2-[4-(2,3-dimethoxybenzyl)-1-(2- | −6.02 |
| | fluorobenzyl)-2-piperazinyl]ethanol | |
| ZiMo12.548 | 4,6-dimethyl-2-oxo-N-[(2-phenoxy- | −6.04 |
| | 3-pyridinyl)methyl]-2H-pyran-5- | |
| | carboxamide | |
| ZiMo12.316 | 1-{1-[(2-isopropyl-1,3-benzoxazol-5- | −6.09 |
| | yl)carbonyl]-4-phenyl-4- | |
| | piperidinyl}ethanone | |
| ZiMo12.152 | ethyl 3-(4-fluorobenzyl)-1-(3- | −6.11 |
| | methoxybenzoyl)-3-piperidinecarboxylate | |
| ZiMo12.107 | 4-[(1-{4-[2-(3-methoxyphenyl)-1- | −6.2 |
| | pyrrolidinyl]-4-oxobutyl}-1H-tetrazol- | |
| | 5-yl)methyl]morpholine | |
| ZiMo12.287 | N-[3-(1H-1,2,3-benzotriazol-1-yl)propyl]- | −6.25 |
| | 1-(2-fluorobenzyl)-1H-1,2,3- | |
| | triazole-4-carboxamide | |
| ZiMo12.464 | N-(3-isopropoxypropyl)-5-{[(6- | −6.53 |
| | methyl-3-pyridinyl)oxy]methyl}-3- | |
| | isoxazolecarboxamide | |
| ZiMo12.353 | 2-[(4-methyl-1,3-thiazol-2-yl)thio]-N-[(2- | −6.57 |
| | phenoxy-3-pyridinyl)methyl]acetamide | |
| ZiMo12.158 | 5-[(4-chloro-3,5-dimethylphenoxy)methyl]- | −6.67 |
| | N-[1-(3-isoxazolyl)ethyl]- | |
| | N-methyl-3-isoxazolecarboxamide | |
| ZiMo12.242 | 5-[(5-isoquinolinyloxy)methyl]-N- | −6.71 |
| | methyl-N-[1-(2-pyridinyl)ethyl]-3- | |
| | isoxazolecarboxamide | |
| ZiMo12.574 | 3-{2-[3-(1,3-benzodioxol-5-ylcarbonyl)- | −6.72 |
| | 1-piperidinyl]-2-oxoethyl}-1,3- | |
| | benzoxazol-2(3H)-one | |
| ZiMo12.597 | N-{1-[5-methyl-1-(1-naphthyl)-1H-pyrazol- | −6.89 |
| | 4-yl]ethyl}-2-(3-pyridinyl)acetamide | |
| ZiMo12.114 | 2-{1-(4-ethoxybenzyl)-4-[(1-ethyl-1H- | −6.94 |
| | pyrazol-4-yl)methyl]-2-piperazinyl}ethanol | |
| ZiMo12.62 | 2-[4-(3,5-difluorobenzyl)-1-(4-methoxy-2,3- | −6.96 |
| | dimethylbenzyl)-2-piperazinyl]ethanol | |
| ZiMo12.514 | N-{[1-(4-biphenylylsulfonyl)-3- | −7.14 |
| | piperidinyl]methyl}-2-furamide | |
| ZiMo12.79 | ethyl 4-[4-(2-fluorobenzyl)-3- | −7.2 |
| | (2-hydroxyethyl)-1-piperazinyl]-1- | |
| | piperidinecarboxylate | |
| ZiMo12.144 | 2-({4-[3-(1-pyrrolidinylcarbonyl)phenoxy]- | −7.32 |
| | 1-piperidinyl}methyl)-1H- | |
| | benzimidazole | |
| ZiMo12.195 | N-[1-(2,5-dimethyl-1,3-thiazol-4- | −7.38 |
| | yl)ethyl]-N′-(3-methoxypropyl)-N- | |
| | methyl[1,2,5]oxadiazolo[3,4- | |
| | b]pyrazine-5,6-diamine | |
| ZiMo12.444 | 1′-[3-(cyclopentyloxy)benzyl]-N- | −7.56 |
| | cyclopropyl-1,4′-bipiperidine-4- | |
| | carboxamide | |
| ZiMo12.500 | 4-methoxy-N-(2-methoxyethyl)-2-({1-[3- | −7.57 |
| | p(methylthio)benzyl]-4- | |
| | iperidinyl}oxy)benzamide | |
| ZiMo12.10 | 3-methyl-N-(1-{1-[(3-phenyl-1H-pyrazol- | −7.72 |
| | 4-yl)methyl]-4-piperidinyl}- | |
| | 1H-pyrazol-5-yl)butanamide | |
| ZiMo12.172 | N-ethyl-2-[1-(3-furylmethyl)-3-oxo- | −7.81 |
| | 2-piperazinyl]-N-[2-(1H-pyrazol-1- | |
| | yl)ethyl]acetamide | |
| ZiMo12.35 | ethyl 1-(2,5-dimethoxybenzoyl)-3-(4- | −7.95 |
| | fluorobenzyl)-3-piperidinecarboxylate | |
| ZiMo12.16 | 2-(1-(2-fluoro-4-methoxybenzyl)-4- | −7.96 |
| | {[1-(2-pyrimidinyl)-1H-pyrrol-2- | |
| | yl]methyl}-2-piperazinyl)ethanol | |
| ZiMo12.349 | ethyl 3-(4-fluorobenzyl)-1-(4- | −7.99 |
| | methoxybenzoyl)-3-piperidinecarboxylate | |
| ZiMo12.346 | 2-{1-(3,5-dimethoxybenzyl)-4-[(5- | −8.01 |
| | methyl-1-propyl-1H-pyrazol-4- | |
| | yl)methyl]-2-piperazinyl}ethanol | |
| ZiMo12.223 | 2-(2-fluoro-4-methoxybenzyl)-7-(2- | −8.03 |
| | methoxyethyl)-2,7-diazaspiro[4.5]decane | |
| ZiMo12.537 | 1-[3-(2-chlorophenyl)-1-(4-fluorophenyl)- | −8.09 |
| | 1H-pyrazol-4-yl]-N-(3- | |
| | isoxazolylmethyl)-N-methylmethanamine | |
| ZiMo12.94 | methyl 5-[7-(3,4-difluorobenzyl)- | −8.12 |
| | 2,7-diazaspiro[4.5]dec-2-yl]-5- | |
| | oxopentanoate | |
| ZiMo12.85 | N-(1′-methyl-1,4′-bipiperidin-3-yl)- | −8.19 |
| | 3-biphenylcarboxamide | |
| ZiMo12.335 | N-methyl-N-[(4-methyl-1,2,5- | −8.21 |
| | oxadiazol-3-yl)methyl]-6-[3- | |
| | (trifluoromethyl)phenyl]imidazo[2,1- | |
| | b][1,3]thiazole-3-carboxamide | |
| ZiMo12.138 | 4-(2-fluorobenzyl)-3-[2-(4-methoxy-1- | −8.42 |
| | piperidinyl)-2-oxoethyl]-2-piperazinone | |
| ZiMo12.511 | (3aS*,6aR*)-3-cyclopentyl-5-[4-(4-hydroxy- | −8.81 |
| | 1-butyn-1-yl)benzyl]hexahydro- | |
| | 2H-pyrrolo[3,4-d][1,3]oxazol-2-one | |
| ZiMo12.472 | N-(2-hydroxybutyl)-1-(2-phenyl-6,7- | −8.86 |
| | dihydro-5H-cyclopenta[d]pyrimidin-4-yl)- | |
| | 4-piperidinecarboxamide | |
| ZiMo12.582 | ethyl 4-[7-(cyclobutylmethyl)-2,7- | −9.04 |
| | diazaspiro[4.5]dec-2-yl]-1- | |
| | piperidinecarboxylate | |
| ZiMo12.421 | N-{[1-(2,3-dihydro-1H-inden-2-yl)- | −9.08 |
| | 4-piperidinyl]methyl}-4,4,4-trifluoro- | |
| | N-(tetrahydro-2-furanylmethyl)butanamide | |
| ZiMo12.575 | N-methyl-N-[(6-methyl-2- | −9.28 |
| | pyridinyl)methyl]-1-(2-phenylethyl)-3- | |
| | piperidinamine | |
| ZiMo12.41 | methyl (2S*,4S*,5R*)-5-(2- | −9.36 |
| | chlorophenyl)-4-{[(3-isoxazolyl- | |
| | methyl)(methyl)amino]carbonyl}- | |
| | 1,2-dimethyl-2-pyrrolidinecarboxylate | |
| ZiMo12.47 | (3,4-dimethoxybenzyl){[2-(1- | −9.55 |
| | pyrrolidinylcarbonyl)imidazo[1,2- | |
| | alpyridin-3-yl]methyl}amine | |
| ZiMo12.470 | N-({1-[2-(4-methoxyphenyl)ethyl]- | −9.55 |
| | 4-piperidinyl}methyl)-N-methyl-2- | |
| | furamide | |
| ZiMo12.326 | N-[2-(1-{[3-(2,3,4-trimethoxyphenyl)- | −9.73 |
| | 1,2,4-oxadiazol-5-yl]methyl}-4- | |
| | piperidinyl)ethyl]acetamide | |
| ZiMo12.37 | 2,6-dimethoxy-4-({[1-(5-methyl- | −9.77 |
| | 1-phenyl-1H-pyrazol-4- | |
| | yl)ethyl]amino}methyl)phenol | |
| ZiMo12.20 | 1-{4-[(cyclooctylamino)methyl]- | −9.9 |
| | 2-methoxyphenoxy}- | |
| | 3-(4-thiomorpholinyl)-2-propanol | |
| ZiMo12.406 | N-methyl-N-[(1-methyl-1H-imidazol- | −10 |
| | 2-yl)methyl]-2-[3-oxo-1-(3- | |
| | phenylpropyl)-2-piperazinyl]acetamide | |
| ZiMo12.521 | 6-(3-chlorophenyl)-N-methyl- | −10.01 |
| | N-[(4-methyl-1,3-thiazol-2- | |
| | yl)methyl]imidazo[2,1-b][1,3]thiazole- | |
| | 3-carboxamide | |
| ZiMo12.467 | 1′-benzyl-N-(2-pyridinylmethyl)- | −10.07 |
| | 1,4′-bipiperidine-4-carboxamide | |
| ZiMo12.503 | 2-[4-[(5-isopropyl-1H-pyrazol-3-yl)methyl]- | −10.07 |
| | 1-(4-methoxy-2,3-dimethylbenzyl)- | |
| | 2-piperazinyl]ethanol | |
| ZiMo12.404 | N-[(1-benzyl-3-piperidinyl)methyl]-6-(4- | −10.11 |
| | morpholinyl)[1,2,5]oxadiazolo[3,4- | |
| | b]pyrazin-5-amine | |
| ZiMo12.423 | ethyl 4-(cyclopropylmethyl)-1-[(2E)- | −10.21 |
| | 3-(2-methoxyphenyl)-2-propen-1- | |
| | yl]-4-piperidinecarboxylate | |
| ZiMo12.428 | N-[(3S)-2-oxo-3-azepanyl]-5-[(5,6,7,8- | −10.23 |
| | tetrahydro-2-naphthalenyloxy)methyl]- | |
| | 3-isoxazolecarboxamide | |
| ZiMo12.274 | 2-{1-(2-ethoxybenzyl)-4-[(1-ethyl- | −10.24 |
| | 1H-pyrazol-4-yl)methyl]-2- | |
| | piperazinyl}ethanol | |
| ZiMo12.435 | 2-{1-(4-methoxy-3-methylbenzyl)- | −10.51 |
| | 4-[(6-methyl-2-pyridinyl)methyl]-2- | |
| | piperazinyl}ethanol | |
| ZiMo12.498 | 2-[(1,5-dimethyl-1H-pyrazol-3- | −10.61 |
| | yl)carbonyl]-7-(3-methylbenzyl)-2,7- | |
| | diazaspiro[4.5]decane | |
| ZiMo12.383 | 2-{[(1-{[3-(3-chlorophenyl)-1-methyl- | −10.62 |
| | 1H-pyrazol-4-yl]methyl}-4- | |
| | piperidinyl)oxy]methyl}pyridine | |
| ZiMo12.146 | N-[2-(1H-imidazol-1-yl)ethyl]- | −10.78 |
| | 2-{[1-(2-pyridinylmethyl)-4- | |
| | piperidinyl]oxy}benzamide | |
| ZiMo12.567 | ethyl 3-(1,3-benzodioxol-5-ylamino)- | −10.79 |
| | 1,4′-bipiperidine-1′-carboxylate | |
| ZiMo12.477 | ethyl 3-benzyl-1-[2- | −10.85 |
| | (methylthio)benzoyl]-3-piperidinecarboxylate | |
| ZiMo12.422 | methyl (2S*,4S*,5R*)-5-(2-chlorophenyl)- | −10.92 |
| | 4-{[(5-isoxazolylmethyl)(methyl) | |
| | amino]carbonyl}-1,2-dimethyl-2- | |
| | pyrrolidinecarboxylate | |
| ZiMo12.609 | N-methyl-N-(1-methyl-4-piperidinyl)- | −10.92 |
| | 2-[3-oxo-1-(3-phenoxybenzyl)-2- | |
| | piperazinyl]acetamide | |
| ZiMo12.267 | 2-[4-(2-fluorobenzyl)-1-(4-methoxy- | −11.07 |
| | 2,3-dimethylbenzyl)-2- | |
| | piperazinyl]ethanol | |
| ZiMo12.42 | 1-[3-({4-[3-oxo-3-(4-phenyl- | −11.26 |
| | 1-piperazinyl)propyl]-1- | |
| | piperidinyl}methyl)phenyl]ethanone | |
| ZiMo12.153 | 5-methyl-N-[(1-{[5-methyl-2- | −11.58 |
| | (2-thienyl)-1,3-oxazol-4-yl]methyl}-3- | |
| | piperidinyl)methyl]-3-isoxazolecarboxamide | |
| ZiMo12.321 | 4-benzyl-1-{3-[1-(3- | −11.58 |
| | cyclohexen-1-ylcarbonyl)-4- | |
| | piperidinyl]propanoyl}piperidine | |
| ZiMo12.373 | N-[1-(2-phenylethyl)-3-piperidinyl]-1- | −11.77 |
| | cyclopentene-1-carboxamide | |
| ZiMo12.270 | N-cyclopentyl-2-methyl-N-({1-[2- | −11.94 |
| | (2-methylphenyl)ethyl]-4- | |
| | piperidinyl}methyl)propanamide | |
| ZiMo12.399 | N-(3,4-dimethoxyphenyl)-1-[2- | −12.02 |
| | (trifluoromethyl)benzoyl]-3- | |
| | piperidinamine | |
| ZiMo12.61 | 3-[2-oxo-2-(1-piperidinyl)ethyl]- | −12.06 |
| | 4-[3-(trifluoromethyl)benzyl]-2- | |
| | piperazinone | |
| ZiMo12.432 | N-{2-[(4-methyl-1,2,5-oxadiazol-3- | −12.19 |
| | yl)oxy]ethyl}-6-phenylimidazo[2,1- | |
| | b][1,3]thiazole-3-carboxamide | |
| ZiMo12.171 | 2-chloro-N-({1-[2-(4-methoxy- | −12.56 |
| | phenyl)ethyl]-4-piperidinyl}methyl)-N- | |
| | methylisonicotinamide | |
| ZiMo12.438 | N-[1-(4-methoxyphenyl)-6,6-dimethyl- | −12.86 |
| | 4,5,6,7-tetrahydro-1H-indazol- | |
| | 4-yl]-2-butynamide | |
| ZiMo12.67 | N-(3-chloro-4-fluorophenyl)-3-{1-[3- | −12.91 |
| | (1H-1,2,4-triazol-1-yl)propanoyl]- | |
| | 4-piperidinyl}propanamide | |
| ZiMo12.570 | 4-({4-[3-oxo-3-(4-phenyl- | −12.92 |
| | 1-piperazinyl)propyl]-1- | |
| | piperidinyl}methyl)phenol | |
| ZiMo12.340 | 2-{[1-(2-fluoro-4-methoxybenzyl)-4- | −12.94 |
| | piperidinyl]oxy}-4-methoxy-N-(2- | |
| | methoxyethyl)benzamide | |
| ZiMo12.583 | 1-{[1-(2-fluorobenzyl)-1H-1,2,3-triazol- | −13.11 |
| | 4-yl]carbonyl}-2-(1,3-thiazol-2- | |
| | yl)piperidine | |
| ZiMo12.542 | 1′-[1-methyl-2-(3-pyridinyl)ethyl]- | −13.12 |
| | N-(2-pyridinylmethyl)-1,4′- | |
| | bipiperidine-4-carboxamide | |
| ZiMo12.476 | N-cyclopropyl-2-[1-(2,3-dihydro-1H- | −13.14 |
| | inden-2-yl)-3-oxo-2-piperazinyl]- | |
| | N-[(1,3,5-trimethyl-1H-pyrazol- | |
| | 4-yl)methyl]acetamide | |
| ZiMo12.523 | 1-(1-adamantylcarbonyl)-N-(4- | −13.24 |
| | fluorophenyl)-3-piperidinamine | |
| ZiMo12.215 | 1-(1H-imidazol-2-ylmethyl)-4-[2-(1- | −13.41 |
| | piperidinylcarbonyl)phenoxy]piperidine | |
| ZiMo12.214 | 2-[(4-methyl-1H-imidazol-2- | −13.43 |
| | yl)carbonyl]-7-(3-phenylpropyl)-2,7- | |
| | diazaspiro[4.5]decane | |
| ZiMo12.224 | (4aS*,8aR*)-2-{2-[(1-acetyl- | −13.6 |
| | 4-piperidinyl)oxy]-5- | |
| | methoxybenzoyl}decahydroisoquinoline | |
| ZiMo12.594 | 2-({[1-(cyclohexylmethyl)-3- | −13.6 |
| | piperidinyl]amino}methyl)-4- | |
| | methoxyphenol | |
| ZiMo12.532 | 1-[1-(2-fluorobenzyl)-2-(methylsulfonyl)- | −13.66 |
| | 1H-imidazol-5-yl]-N-[(5- | |
| | fluoro-1H-indol-2-yl)methyl]-N- | |
| | methylmethanamine | |
| ZiMo12.568 | 2-{1-(3,5-dimethoxybenzyl)-4-[3- | −13.71 |
| | (methylthio)benzyl]-2-piperazinyl}ethanol | |
| ZiMo12.377 | N-(1-benzyl-3-pyrrolidinyl)-3-{1- | −13.77 |
| | [(5-methyl-1H-pyrazol-3-yl)carbonyl]- | |
| | 4-piperidinyl}propanamide | |
| ZiMo12.465 | 6-{4-[(1-methyl-1H-imidazol-2- | −13.79 |
| | yl)methyl]-1-piperazinyl}-N-(2- | |
| | thienylmethyl)[1,2,5]oxadiazolo[3,4- | |
| | b]pyrazin-5-amine | |
| ZiMo12.410 | N~2~,N~2 ~-dimethyl-N~5 | −13.88 |
| | ~-(1-propyl-4-piperidinyl)-5,6,7,8- | |
| | tetrahydro-2,5-quinazolinediamine | |
| ZiMo12.466 | 3-[1-(tetrahydro-2H-pyran-4- | −14.06 |
| | ylcarbonyl)-4-piperidinyl]-N-[3- | |
| | (trifluoromethyl)benzyl]propanamide | |
| ZiMo12.387 | 2-isonicotinoyl-7-(3-phenylpropyl)- | −14.1 |
| | 2,7-diazaspiro[4.5]decane | |
| ZiMo12.8 | 1-{[3-(diphenylmethyl)-1,2,4-oxadiazol- | −14.12 |
| | 5-yl]methyl}-4-(2-furoyl)-1,4- | |
| | diazepane | |
| ZiMo12.92 | 5-[(4-chloro-3,5-dimethyl- | −14.13 |
| | phenoxy)methyl]-N-(1,4-dioxan-2-ylmethyl)- | |
| | 3-isoxazolecarboxamide | |
| ZiMo12.46 | N-({1-[2-(4-chlorophenyl)ethyl]-3- | −14.15 |
| | piperidinyl}methyl)-4,4,4-trifluoro-N- | |
| | methylbutanamide | |
| ZiMo12.232 | 3-[1-(tetrahydro-2-furanylcarbonyl)- | −14.25 |
| | 4-piperidinyl]-N-[3- | |
| | (trifluoromethyl)benzyl]propanamide | |
| ZiMo12.518 | 5-{[(2-methyl-1,3-benzothiazol-5- | −14.62 |
| | yl)oxy]methyl}-N-[1-(1,3-thiazol-2- | |
| | yl)ethyl]-3-isoxazolecarboxamide | |
| ZiMo12.344 | N-({1-[2-(2-methoxyphenyl)ethyl]- | −14.73 |
| | 4-piperidinyl}methyl)-N-methyl-3- | |
| | (methylthio)propanamide | |
| ZiMo12.154 | 2-[1-(2-ethoxybenzyl)-3-oxo-2- | −15.06 |
| | piperazinyl]-N-methyl-N-(4- | |
| | pyrimidinylmethyl)acetamide | |
| ZiMo12.276 | 2-[1-(4-methoxy-2,3-dimethylbenzyl)- | −15.09 |
| | 3-oxo-2-piperazinyl]-N-methyl- | |
| | N-[(1-methyl-1H-pyrazol-4- | |
| | yl)methyl]acetamide | |
| ZiMo12.56 | methyl 1-{[2-(benzylsulfonyl)-1- | −15.1 |
| | isobutyl-1H-imidazol-5-yl]methyl}-2- | |
| | piperidinecarboxylate | |
| ZiMo12.378 | 1-{[1-(2-fluorobenzyl)-2-(methylsulfonyl)- | −15.1 |
| | 1H-imidazol-5-yl]methyl}-3- | |
| | methylpiperidine | |
| ZiMo12.169 | N-ethyl-N-[(1′-methyl-1,4′-bipiperidin-4- | −15.33 |
| | yl)methyl]-4-biphenylcarboxamide | |
| ZiMo12.333 | N-methyl-N-{[3-(2-pyridinyl)-1,2,4- | −15.36 |
| | oxadiazol-5-yl]methyl}cyclohexanamine | |
| ZiMo12.134 | methyl 3-{[3-(3-chlorobenzoyl)-1- | −15.38 |
| | piperidinyl]sulfonyl}-2-thiophenecarboxylate | |
| ZiMo12.501 | 1-[4-({4-[3-oxo-3-(4-phenyl- | −15.51 |
| | 1-piperazinyl)propyl]-1- | |
| | piperidinyl}methyl)-2-thienyllethanone | |
| ZiMo12.206 | N-ethyl-N-({1-[2-(2-methyl- | −15.52 |
| | phenyl)ethyl]-4-piperidinyl}methyl)-2- | |
| | thiophenecarboxamide | |
| ZiMo12.162 | ethyl 3-(2-phenoxyethyl)-1-(2- | −15.54 |
| | pyridinylmethyl)-3-piperidinecarboxylate | |
| ZiMo12.122 | 1-(2,3-dimethoxyphenyl)-2-(1H-imidazol- | −15.83 |
| | 4-ylcarbonyl)-2,3,4,9-tetrahydro- | |
| | 1H-beta-carboline | |
| ZiMo12.475 | 2-(1-{[2-(ethylsulfonyl)-1-(2-phenylethyl)- | −15.83 |
| | 1H-imidazol-5-yl]methyl}-2- | |
| | pyrrolidinyl)pyridine | |
| ZiMo12.175 | N-(3,5-dimethoxyphenyl)-3-(1-{[2- | −15.86 |
| | (dimethylamino)-1,3-thiazol-5- | |
| | yl]methyl}-3-piperidinyl)propanamide | |
| ZiMo12.179 | 3-(1-cycloheptyl-3-piperidinyl)- | −16.13 |
| | N-(2-fluorophenyl)propanamide | |
| ZiMo12.481 | N-ethyl-2-methyl-N-({1-[2-(2- | −16.28 |
| | methylphenyl)ethyl]-4-piperidinyl}methyl)- | |
| | 1,3-thiazole-4-carboxamide | |
| ZiMo12.211 | N-{[8-methyl-2-(4-morpho- | −16.45 |
| | linylcarbonyl)imidazo[1,2-a]pyridin-3- | |
| | yl]methyl}-2-(1-methyl-2- | |
| | pyrrolidinyl)ethanamine | |
| ZiMo12.161 | N-(3-chloro-4-fluorophenyl)- | −16.74 |
| | 3-[1-(3-methyl-2-furoyl)-4- | |
| | piperidinyl]propanamide | |
| ZiMo12.15 | 1-[3-(2-fluoro-4-methoxyphenyl)-1- | −16.77 |
| | phenyl-1H-pyrazol-4-yl]-N-methyl- | |
| | N-(2-pyrazinylmethyl)methanamine | |
| ZiMo12.70 | N-[4-(1H-pyrazol-1-yl)phenyl]- | −16.89 |
| | 1-(3-pyridinylmethyl)-2- | |
| | piperidinecarboxamide | |
| ZiMo12.55 | N-(1,4-dioxan-2-ylmethyl)-N-methyl-5-{[3- | −17.34 |
| | (trifluoromethyl)phenoxy]methyl}- | |
| | 1H-pyrazole-3-carboxamide | |
| ZiMo12.272 | N-(2,4-dimethoxyphenyl)-3-{1-[(2- | −17.65 |
| | methoxy-3-pyridinyl)carbonyl]-4- | |
| | piperidinyl}propanamide | |
| ZiMo12.334 | 1-(1-cyclopentyl-4-piperidinyl)- | −18 |
| | N-(2,3-dimethoxybenzyl)-N- | |
| | (tetrahydro-2-furanylmethyl)methanamine | |
| ZiMo12.433 | 2-methyl-N-[(2-{methyl[2- | −18.29 |
| | (2-pyridinyl)ethyl]amino}-3- | |
| | pyridinyl)methyl]-1,3-thiazole-4-carboxamide | |
| ZiMo12.429 | 2-({[1-({3-[4-(trifluoromethyl)phenyl]- | −18.3 |
| | 1,2,4-oxadiazol-5-yl}methyl)-4- | |
| | piperidinyl]oxy}methyl)pyridine | |
| ZiMo12.563 | ethyl 4-(4-methoxybenzyl)-1-[(2-methyl- | −18.41 |
| | 1-benzofuran-5-yl)carbonyl]- | |
| | 4-piperidinecarboxylate | |
| ZiMo12.245 | 1-[(7-chloro-3,5-dimethyl-1- | −18.43 |
| | benzofuran-2-yl)carbonyl]-4-(1- | |
| | piperidinylcarbonyl)piperidine | |
| ZiMo12.22 | 1′-[2-(1H-benzimidazol-1- | −18.53 |
| | yl)propanoyl]-4-(1-pyrrolidinylcarbonyl)- | |
| | 1,4′-bipiperidine | |
| ZiMo12.167 | 2-(1-(2-fluorobenzyl)-4-{[1-(4- | −18.61 |
| | methylphenyl)-1H-pyrazol-4-yl]methyl}- | |
| | 2-piperazinyl)ethanol | |
| ZiMo12.325 | N-phenyl-1-[4-(2-thienyl)butanoyl]- | −18.79 |
| | 3-piperidinamine | |
| ZiMo12.341 | N-benzyl-1-(2-chlorobenzyl)-N-methyl- | −18.95 |
| | 1H-1,2,3-triazole-4-carboxamide | |
| ZiMo12.282 | N-cyclopropyl-4-methoxy-2-{[1-(4- | −19.37 |
| | methylbenzyl)-4-piperidinyl]oxy}benzamide | |
| ZiMo12.394 | 2-ethoxy-6-{[4-(2-ethoxybenzyl)-3-(2- | −19.63 |
| | hydroxyethyl)-1-piperazinyl]methyl}phenol | |
| ZiMo12.388 | 2-[1-(3,5-dimethoxybenzyl)-4-(2,5- | −19.88 |
| | dimethylbenzyl)-2-piperazinyl]ethanol | |
| ZiMo12.280 | 1-{3-[1-(2-fluoro-5-methoxybenzyl)-4- | −19.92 |
| | piperidinyl]propanoyl}-4-phenylpiperazine | |
| ZiMo12.425 | 3-[1-(2-chloro-6-fluorobenzyl)-4-piperidinyl]- | −19.96 |
| | N-[(1-ethyl-2-pyrrolidinyl)methyl]pro- | |
| | panamide | |
| ZiMo12.247 | 4-(2-phenylethyl)-1-{[1-(tetrahydro- | −20.6 |
| | 2-furanylmethyl)-1H-1,2,3-triazol- | |
| | 4-yl]carbonyl}piperidine | |
| ZiMo12.339 | 1′-(2-phenylethyl)-N-(2,2,2-trifluoroethyl)- | −20.6 |
| | 1,4′-bipiperidine-3-carboxamide | |
| ZiMo12.273 | N-(4-methoxyphenyl)-1-{[(5-methyl-1H- | −20.79 |
| | benzimidazol-2-yl) thio]acetyl}- | |
| | 3-piperidinamine | |
| ZiMo12.555 | N-cyclopropyl-4-methoxy-2-[(1-{[2- | −21.09 |
| | (methylthio)-5-pyrimidinyl]methyl}- | |
| | 4-piperidinyl)oxy]benzamide | |
| ZiMo12.196 | 4-(4-methoxy-3-methylbenzyl)-3- | −21.29 |
| | [2-(4-methoxy-1-piperidinyl)-2- | |
| | oxoethyl]-2-piperazinone | |
| ZiMo12.240 | N-cyclopropyl-1′-[3-(1H-pyrazol- | −21.32 |
| | 1-yl)benzyl]-1,4′-bipiperidine-4- | |
| | carboxamide | |
| ZiMo12.90 | 4-(benzyloxy)-1-{[3-(4-methoxyphenyl)- | −21.43 |
| | 1,2,4-oxadiazol-5-yl]methyl}piperidine | |
| ZiMo12.558 | 2-(3-{3-[(5-chloro-2-methoxyphenyl)amino]- | −22.65 |
| | 3-oxopropyl}-1-piperidinyl)nicotinamide | |
| ZiMo12.268 | N-(5-chloro-2-methoxyphenyl)- | −23.36 |
| | 3-(1-{[5-(methoxymethyl)-2- | |
| | furyl]methyl}-3-piperidinyl)propanamide | |
| ZiMo12.7 | 1-(5-chloro-2-methylphenyl)-4-{[3- | −23.53 |
| | (2-pyridinyl)-1,2,4-oxadiazol-5- | |
| | yl]methyl}piperazine | |
| ZiMo12.473 | 7-(2,3-difluorobenzyl)-2-[(1-methyl- | −23.6 |
| | 1H-imidazol-2-yl)methyl]-2,7- | |
| | diazaspiro[4.5]decane | |
| ZiMo12.159 | 3-[(1-acetyl-4-piperidinyl)oxy]-4-methoxy- | −24.36 |
| | N-(1-phenylethyl)benzamide | |
| ZiMo12.365 | (3R*,4R*)-1-{[5-(methoxymethyl)- | −24.49 |
| | 2-furyl]methyl}-4-[4-(2-pyridinyl)-1- | |
| | piperazinyl]-3-piperidinol | |
| ZiMo12.219 | 1-{[2-(ethylsulfonyl)-1-(3-phenylpropyl)-1H- | −24.55 |
| | imidazol-5-yl]methyl}-4-methoxypiperidine | |
| ZiMo12.190 | N-methyl-N-(2-pyrazinylmethyl)-6-[3- | −24.57 |
| | (trifluoromethyl)phenyl]imidazo[2,1- | |
| | b][1,3]thiazole-3-carboxamide | |
| ZiMo12.369 | 7-(2,3-difluorobenzyl)-2-(4,4,4- | −24.67 |
| | trifluorobutyl)-2,7-diazaspiro[4.5]decane | |
| ZiMo12.354 | N-[2-(3-furoyl)-1,2,3,4-tetrahydro-7- | −24.73 |
| | isoquinolinyl]-1-phenylcyclopropane- | |
| | carboxamide | |
| ZiMo12.324 | 2-(cyclopentylcarbonyl)-7-(2-fluoro-5- | −25.46 |
| | methoxybenzyl)-2,7-diazaspiro[4.5]decane | |
| ZiMo12.292 | N-[3-(1H-indol-2-yl)phenyl]-1-propionyl- | −26.33 |
| | 3-piperidinecarboxamide | |
| ZiMo12.439 | N-[4-({3-[(3-fluorophenyl)amino]-1- | −26.71 |
| | piperidinyl}carbonyl)phenyl]acetamide | |
| ZiMo12.262 | 1-({5-[(4-chloro-3,5- | −27.3 |
| | dimethylphenoxy)methyl]- | |
| | 3-isoxazolyl}carbonyl)azocane | |
| ZiMo12.69 | 1-(1-cyclopentyl-4-piperidinyl)-N- | −27.37 |
| | (2,4-difluorobenzyl)-N-(tetrahydro- | |
| | 2-furanylmethyl)methanamine | |
| ZiMo12.328 | 4-{[7-(2-phenylethyl)-2,7- | −27.52 |
| | diazaspiro[4.5]dec-2-yl]carbonyl}-1 (2H)- | |
| | phthalazinone | |
| ZiMo12.459 | 1-(2,3-dihydro-1H-inden-2-yl)- | −27.71 |
| | N-[2-(3-pyridinyloxy)phenyl]-3- | |
| | piperidinecarboxamide | |
| ZiMo12.488 | 1′-[(6-methyl-2-pyridinyl)methyl]- | −28.51 |
| | N-(2-pyridinylmethyl)-1,4′- | |
| | bipiperidine-4-carboxamide | |
| ZiMo12.452 | (3R*,4R*)-4-(1-azepanyl)-1-[2-(2- | −28.89 |
| | hydroxyethoxy)benzyl]-3-piperidinol | |
| ZiMo12.220 | 3-[1-(5-chloro-2-hydroxybenzyl)- | −28.92 |
| | 4-piperidinyl]-N-[(1-ethyl-2- | |
| | pyrrolidinyl)methyl]propanamide | |
| ZiMo12.513 | 1-{4-[(2,3-dihydro-1H-inden-2- | −29.2 |
| | ylamino)methyl]-2-methoxyphenoxy}- | |
| | 3-(1-pyrrolidinyl)-2-propanol | |
| ZiMo12.336 | N-{[8-methyl-2-(4-morpho- | −29.47 |
| | linylcarbonyl)imidazo[1,2-a]pyridin-3- | |
| | yl]methyl}-2-(4-pyridinyl)ethanamine | |
| ZiMo12.588 | ethyl 3-(2-fluorobenzyl)-1-[(1-isopropyl- | −30.58 |
| | 1H-pyrazol-4-yl)methyl]-3- | |
| | piperidinecarboxylate | |
| ZiMo12.479 | (3aS*,5S*,9aS*)-2-(3-chlorobenzyl)-5-(3,5- | −31.02 |
| | dimethoxyphenyl)hexahydro-7H- | |
| | pyrrolo[3,4-g]pyrrolizin-1(2H)-one | |
| ZiMo12.371 | methyl 4-{[7-(cyclohexylmethyl)-2,7- | −31.51 |
| | diazaspiro[4.5]dec-2-yl]carbonyl}benzoate | |
| ZiMo12.269 | 1-(diethylamino)-3-(2-methoxy-5-{[(4- | −32.74 |
| | methoxybenzyl)amino]methyl}phenoxy)- | |
| | 2-propanol | |
| ZiMo12.111 | 2-[(3,5-dimethyl-4-isoxazolyl)acetyl]- | −33.89 |
| | 1-isobutyl-2,3,4,9-tetrahydro- | |
| | 1H-beta-carboline | |
| ZiMo12.204 | ethyl 3-benzyl-1-(1H-indol-5-ylcarbonyl)- | −34.03 |
| | 3-piperidinecarboxylate | |
| ZiMo12.545 | N-methyl-N-[2-(4-morpholinyl)ethyl]- | −34.53 |
| | 4-{[1-(2-pyridinylmethyl)-4- | |
| | piperidinyl]oxy}benzamide | |
| ZiMo12.461 | N-(3,5-dimethoxyphenyl)-3-(1-{[2- | −34.93 |
| | (methylthio)-5-pyrimidinyl]methyl}- | |
| | 3-piperidinyl)propanamide | |
| ZiMo12.380 | 3-(2-methoxyphenyl)-N-[2-(4- | −35.22 |
| | pyridinylmethyl)-1,2,3,4-tetrahydro-7- | |
| | isoquinolinyl]propanamide | |
| ZiMo12.554 | N-(sec-butyl)-6-(2-chloro- | −35.3 |
| | phenyl)imidazo[2,1-b][1,3]thiazole-3- | |
| | carboxamide | |
| ZiMo12.393 | (3′R*,4′R*)-1′-[3-(2-furyl)benzyl]- | −36.69 |
| | 1,4′-bipiperidine-3′,4-diol | |
| ZiMo12.357 | 1-(4-fluorobenzyl)-3-hydroxy-3-({[(3- | −37.5 |
| | methyl-2-pyridinyl)methyl]amino}methyl)- | |
| | 2-piperidinone | |
| ZiMo12.230 | 7-(2,3-difluorobenzyl)-2- | −39.21 |
| | (1-propyl-4-piperidinyl)-2,7- | |
| | diazaspiro[4.5]decane | |
| ZiMo12.288 | 2-(2-fluorophenyl)-N-{2-[(2-methyl- | −41.67 |
| | 1,3-thiazol-5-yl)methyl]-1,2,3,4- | |
| | tetrahydro-7-isoquinolinyl}acetamide | |
| ZiMo12.581 | 3-chloro-N-[(4-methyl-1H- | −41.82 |
| | imidazol-2-yl)methyl]-4-{[1-(methylsulfonyl)- | |
| | 4-piperidinyl]oxy}benzamide | |
| ZiMo12.389 | ethyl 1-(1,4-dithiepan-6-yl)-3-(3- | −43.9 |
| | methoxybenzyl)-3-piperidinecarboxylate | |
| ZiMo12.491 | ethyl 3-(2-fluorobenzyl)-1-(1- | −45.32 |
| | isoquinolinylcarbonyl)-3- | |
| | piperidinecarboxylate | |
| ZiMo12.225 | 3-chloro-4-fluoro-N-({1-[2- | −49.16 |
| | (2-methoxyphenyl)ethyl]-4- | |
| | piperidinyl}methyl)-N-methylbenzamide | |
| ZiMo12.584 | ethyl 3-(2-fluorobenzyl)-1-{[2- | −52.13 |
| | (methylthio)-5-pyrimidinyl]methyl}-3- | |
| | piperidinecarboxylate | |
| ZiMo12.392 | (3aS*,5S*,9aS*)-2-(3-chlorobenzyl)- | −52.35 |
| | 5-(3-methoxyphenyl)hexahydro- | |
| | 7H-pyrrolo[3,4-g]pyrrolizin-1(2H)-one | |
| ZiMo12.510 | 1-(5-ethyl-2-pyridinyl)-N-methyl-N- | −56.4 |
| | {[3-(3,4,5-trimethoxyphenyl)-1H- | |
| | pyrazol-4-yl]methyl}methanamine | |
| ZiMo12.517 | 2-(1,3-benzodioxol-4-ylmethyl)-7-(2,2- | −58.65 |
| | dimethylpropyl)-2,7-diazaspiro[4.5]decane | |
| ZiMo12.66 | 4-(4-ethoxybenzyl)-3-{2-oxo-2-[4- | −63.25 |
| | (2-pyrazinyl)-1-piperazinyl]ethyl}-2- | |
| | piperazinone | |
| ZiMo12.4 | 2-{3-oxo-1-[3-(trifluoro- | −63.63 |
| | methyl)benzyl]-2-piperazinyl}-N-[3-(1H- | |
| | pyrazol-1-yl)propyl]acetamide | |
| ZiMo12.130 | 4-{[7-(cyclohexylmethyl)-2,7- | −68.92 |
| | diazaspiro[4.5]dec-2- | |
| | yl]carbonyl}benzaldehyde | |
| ZiMo12.3 | (3aS*,5S*,9aS*)-2-(3-chlorobenzyl)-5-(2,6- | −83.25 |
| | dimethoxyphenyl)hexahydro- | |
| | 7H-pyrrolo[3,4-g]pyrrolizin-1(2H)-one | |

At least the following is claimed:

1. A ZIP12 antagonist, comprising one of the following structures: 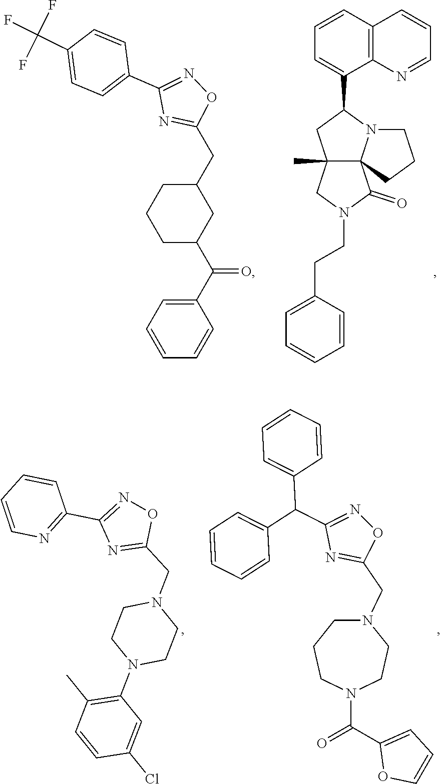
or a pharmaceutically-acceptable salt thereof.

2. A pharmaceutical composition, comprising one or more ZIP12 antagonists of claim 1 and a pharmaceutically-acceptable carrier.

3. A method of increasing zinc in the synaptic cleft, comprising:
administering one or more ZIP12 antagonists of claim 1, or a pharmaceutically-acceptable salt thereof, to a subject in need thereof.

4. The method of claim 3, wherein the subject in need thereof has, or is suspected of having, dysregulated ZIP12 expression.

5. The method of claim 3, wherein the one or more ZIP12 antagonists are administered in an effective amount in increase Zn²⁺ concentration in a synaptic cleft of a subject.

6. The method of claim 3, wherein the one or more ZIP12 antagonists are administered at a concentration of about 1 μM to about 50 μM.

7. The method of claim 6, wherein the one or more ZIP12 antagonists are administered at a concentration of 10 μM.

8. The method of claim 3, wherein the subject in thereof has a neuropsychiatric disorder caused by dysregulated ZIP12 expression, a neurodegenerative disorder caused by dysregulated ZIP12 expression, a neurodevelopmental disorder caused by dysregulated ZIP12 expression, a cancer caused by dysregulated ZIP12 expression, or inflammation caused by dysregulated ZIP12 expression.

9. The method of claim 8, wherein the one or more ZIP12 antagonists are administered in an effective amount to alleviate one or more symptoms of the neurodegenerative disorder caused by dysregulated ZIP12 expression, the neurodevelopmental disorder caused by dysregulated ZIP12 expression, the cancer caused by dysregulated ZIP12 expression, or the inflammation caused by dysregulated ZIP12 expression.

10. The method of claim 8, wherein the neuropsychiatric disorder caused by dysregulated ZIP12 expression is schizophrenia.

11. The method of claim 8, wherein the neurodegenerative disorder caused by dysregulated ZIP12 expression is Alzheimer's Disease (AD) or Parkinson's Disease (PD).

12. The method of claim 8, wherein the neurodevelopmental disorder caused by dysregulated ZIP12 expression is an autism spectrum disorder (ASD).

13. The method of claim 3, wherein the one or more ZIP12 antagonists are delivered to the central nervous system of the subject.

14. The method of claim 3, wherein the one or more ZIP12 antagonists are administered intracerebrally, intraventricularly, or intrathecally.

15. A kit, comprising:
one or more ZIP12 agonists or pharmaceutically-acceptable salts thereof, one or more ZIP12 antagonists or pharmaceutically acceptable salts thereof, or both; and
instructions for use.

16. The kit of claim 15, further comprising a polynucleotide vector encoding a functional ZIP12 protein.

17. The kit of claim 15, wherein the one or more ZIP12 agonists, one or more ZIP12, antagonists, or both, are provided in a dosage unit form.

18. The kit of claim 17, wherein the dosage unit form is a concentration of 10 μM.

19. The kit of claim 15, wherein the one or more ZIP12 antagonists are one or more ZIP12 antagonists of claim 1.

20. The kit of claim 15, further comprising a source of Zn²⁺.


SEQUENCE LISTING (4 sequences)

SEQ ID NO: 1 - protein, 10 aa, Homo sapiens
QDEDSYFLSE

SEQ ID NO: 2 - protein, 10 aa, Homo sapiens
KETDDILAFT

SEQ ID NO: 3 - protein, 10 aa, Homo sapiens
IIALSLQGVC

SEQ ID NO: 4 - protein, 10 aa, Homo sapiens
LGRKALPSPD